(19) United States
(12) Patent Application Publication
(10) Pub. No.: US 2011/0158995 A1
(43) Pub. Date: Jun. 30, 2011

(54) Multi-Specific Binding Proteins Targeting B Cell Disorders

(75) Inventors: Philip Tan, Edmonds, WA (US); Laura Sue Grosmaire, Hobart, WA (US); Peter Robert Baum, Seattle, WA (US); Peter Armstrong Thompson, Bellevue, WA (US)

(73) Assignee: RENAULT S.A.S, BOUIOGNE-BILLANCOURT, FR (unknown)

(21) Appl. No.: 13/056,141

(22) PCT Filed: Jul. 28, 2009
(86) PCT No.: PCT/US09/51990

§ 371 Date: Feb. 18, 2011

(30) Foreign Application Priority Data
Jul. 28, 2008 (US) 61/084209

(51) Int. Cl. A61K 39/395; C12P 21/04; C12N 5/10; C12N 15/63; C07K 16/00; C07H 21/04; A61P 19/02; A61P 29/00; A61P 37/00

(52) U.S. Cl. 424/134.1; 435/69.7; 435/325; 435/320.1; 530/387.3; 536/23.4

(57) ABSTRACT

This disclosure provides a multi-specific fusion protein composed of a CD72-ligand binding domain and another binding domain specific for a heterologous target, such as a B-cell specific protein. The multi-specific fusion protein may also include an intervening domain that separates the other domains. This disclosure also provides polynucleotides encoding the multi-specific fusion proteins, compositions of the fusion proteins, and methods of using the multi-specific fusion proteins and compositions.

### TECHNICAL FIELD

This disclosure relates generally to the field of multi-specific binding molecules and therapeutic applications thereof and more specifically to fusion proteins composed of a CD72-ligand binding domain and another binding domain specific for a heterologous B cell specific target, such as a FCRL1-6, CD19, CD20, CD22, CD32b, CD37, CD79a, CD79b, CD267 or CD269, as well as compositions and therapeutic uses thereof.

### BACKGROUND

The human immune system generally protects the body from damage by foreign substances and pathogens. One way in which the immune system protects the body is by producing specialized cells, referred to as B lymphocytes or B-cells. B-cells produce antibodies that bind to and, in some instances, mediate destruction of a foreign substance or pathogen.

B-cell antigen receptors (BCRs) are important in the development of an antibody response and in regulating B-cell development (see, e.g., Gauld et al. (2002) Science 296:1641; Niiro and Clark (2002) Nat. Rev. Immunol. 2:945). BCR signals can influence cell death, survival, proliferation, and differentiation, so inhibitory signals exist to prevent excessive and sometimes harmful antibody responses (Ravetch and Lanier (2000) Science 290:84). One such inhibitor is CD72, a 45 KDa type II membrane protein containing an extracellular C-type lectin-like domain and a cytoplasmic immunoreceptor tyrosine-based inhibitory motif (ITIM). CD72 negatively regulates BCR signals by recruiting the tyrosine phosphatase SHP-1 to its ITIM (Adachi et al. (1998) J. Immunol. 160:4662). The CD72 inhibition of BCR signaling is reversed by the transmembrane semaphorin CD100 (also known as Sema4D), which is a natural ligand of CD72 (see Kumanogoh et al. (2000) Immunity 13:621; Kumanogoh and Kikutani (2001) Trends Immunol. 22:670). The interaction between the ligand-receptor pair of CD100 and CD72 is considered low affinity (i.e., approximately 3×10⁻⁷ M). Another receptor for CD100 has been identified as Plexin B1, expressed by epithelial cells, which specifically binds CD100 with high affinity (i.e., approximately 1×10⁻⁹ M).

An additional ligand for CD72 is the scavenger receptor family molecule CD5. CD5 is a 67 KDa cell surface glycoprotein expressed on all T-lymphocytes and on some B-cells during development and after malignant transformation to B-cell chronic lymphocytic leukemia (B-CLL). CD5 acts as a co-receptor in the stimulation of T-cell growth and is a natural ligand for murine and human CD72 (Van de Velde et al. (1991) Nature 351:662). The strength of CD72/CD5 has not been described, but it is common for members of the scavenger receptor family to have a number of ligands normally of modest to low affinity.

All B-cell compartments in tissues express CD72, including pulpa macrophages of the spleen and Kupffer cells of the liver, whereas CD100 is expressed on a subset of developing B cells. In peripheral blood and bone marrow, CD72 appears to be present on all B-lymphocytes except for plasma cells (see Wu and Bondada (2002) Immunology Res. 25:155). Expression of CD72 has also been reported in a subset of T-cells (Robinson et al. (1993) J. Immunol. 151:4764) and may mediate aspects of B-cell/T-cell interaction. CD100 is also expressed constitutively on T cells and NK cells, and has recently been found on platelets. On B cells expressing both CD100 and CD72, the two appear to be segregated from each other in the membrane with CD100 being associated with the BCR.

In some instances though, B-cell signaling can go awry and disease results. There are several autoimmune and inflammatory diseases that involve B-cells in their pathology. Such diseases result from inappropriate B-cell antigen presentation to T-cells or other pathways involving B-cells. B-cell signaling has been linked to autoimmune disorders such as systemic lupus erythematosus (SLE) (Hitomi et al. (2004) Hum. Mol. Genet. 13:2907) and idiopathic thrombocytopenic purpura (ITP) (Xu et al. (2007) J. Clin. Immunol. 28:214), as well as to numerous cancers involving uncontrolled proliferation of B-cells. For example, CD72 was identified as a marker for progenitor B-cell leukemias (Schwarting et al. (1992) Am. J. Hematol. 41:151), and CD100 is found on malignant T cells, on malignant B-cells, such as in Burkitt's lymphoma and B-CLL (Circosta et al. (2001) Blood, 98:360a; Granziero et al. (2003) Blood 101:1962), and on lymphoma cells (Dorfman et al. (1998) Am. J. Pathol. 153:255), and is involved in neuroinflammatory disease (Giraudon (2005) Neuro. Molecular Medicine 7:207).

### BRIEF DESCRIPTION OF THE FIGURES

FIG. 1 shows SDS-PAGE characterization of multi-specific fusion proteins containing a CD72 ectodomain fused to a CD79b binding domain (referred to as X7972).

FIG. 2 shows that multi-specific fusion proteins containing a CD72 ectodomain fused to a CD79b binding domain could bind to target CD79b.

FIG. 3 shows that multi-specific fusion proteins containing a CD72 ectodomain fused to a CD79b binding domain could bind to target CD100.

FIG. 4 shows that multi-specific fusion proteins containing a CD72 ectodomain fused to a CD79b binding domain could bind to both targets CD79b and CD100 simultaneously.

FIG. 5 shows that multi-specific fusion proteins containing a CD72 ectodomain fused to either a CD19 or CD37 binding domain (referred to as X1972 and X3772, respectively) can bind to BJAB B-cells.

FIG. 6 shows that multi-specific fusion proteins containing a CD72 ectodomain fused to a CD79b binding domain can bind to Ramos cells.

FIG. 7 shows that multi-specific fusion proteins containing a CD72 ectodomain fused to either a CD19 or CD37 binding domain (referred to as X1972 and X3772, respectively) have CDC activity in 10% human serum.

FIG. 8 shows that a multi-specific fusion protein containing a CD72 ectodomain fused to a CD37 binding domain (X3772) inhibits Rec-1 B-cell growth.

FIG. 9 shows that multi-specific fusion proteins containing a CD72 ectodomain fused to a CD19 binding domain (X1972) inhibit Rec-1 B-cell growth.

FIG. 10 shows that X3772 inhibits cell growth of rituximab resistant Rec-1 B-cells.

FIG. 11 shows that X3772 inhibits cell growth of wild-type Rec-1 B-cells.

FIG. 12 shows that X3772 did not affect growth of non-B cell Jurkat cells.

FIG. 13 shows that X1972 inhibits cell growth of BJAB B-cells.

FIG. 14 shows that multi-specific fusion proteins containing a CD72 ectodomain fused to a CD37 binding domain inhibit cell growth of BJAB B-cells.

FIG. 15 shows that multi-specific fusion proteins containing a CD72 ectodomain fused to a CD79b binding domain cell growth of DOHH2 cells.

FIG. 16 shows that fusion protein X7972.1, which contains a CD72 ectodomain fused to a CD79b binding domain, inhibits growth of DOHH2 cells, whereas the CD72 ectodomain alone, the CD79b binding domain alone, or a combination of the CD72 ectodomain with the CD79b binding domain did not inhibit DOHH2 growth.

FIG. 17 shows that fusion proteins containing a CD72 ectodomain fused to a CD79b binding domain inhibit growth of Ramos cells.

FIG. 18 shows that a variant of the X3772 multi-specific fusion protein inhibits cell growth of rituximab-resistant DOHH2 B-cells

FIG. 19 shows that fusion proteins containing a CD72 ectodomain fused to a CD37 binding domain inhibit growth of rituximab-resistant DOHH-2 cells.

FIG. 20 shows that fusion proteins containing a CD72 ectodomain fused to a CD19 binding domain inhibited growth of rituximab-resistant DOHH-2 cells.

FIGS. 21 and 22 shows that variants of a fusion protein containing a CD72 ectodomain fused to a CD37 binding domain (X3772.1, X3772.2, X3772.3) were more potent in inducing growth inhibition of rituximab-resistant DOHH2 cells than X3772.

FIG. 23 shows that a fusion protein containing a CD72 ectodomain fused to a CD79b binding domain inhibited growth of a rituximab-resistant DOHH2 cell line.

FIG. 24 shows that X3772 linker variants mediate ADCC on Ramos cells to different extents.

FIGS. 25 A and B show that X7972.1 has enhanced ADCC activity against DOHH-2 cells when expressed by cells treated with castanospermine or kifunensine.

FIGS. 26A and B show the effects of X7972.1 on DOHH2 cell cycle at 12 hours and 24 hours, respectively.

### DETAILED DESCRIPTION

The present disclosure makes possible the depletion or modulation of cells associated with aberrant CD72 activity, such as B cells, by providing multi-specific fusion proteins that bind both a CD72 ligand and a second target other than a CD72 ligand, such as a FCRL1-6, CD19, CD20, CD22, CD32b, CD37, CD79a, CD79b, CD267 or CD269. In certain embodiments, a multi-specific fusion protein comprises a first and second binding domain, a first and second linker, and an intervening domain, wherein one end of the intervening domain is fused via a linker to a first binding domain that is a CD72 ectodomain (e.g., an extracellular domain, a C-type lectin domain, or the like) and at the other end fused via a linker to a second binding domain that is a B-cell binding domain, such as an immunoglobulin variable region that is specific for a B-cell protein (e.g., FCRL1-6, CD19, CD20, CD22, CD32b, CD37, CD79a, CD79b, CD267 or CD269). In some embodiments, less than an entire CD72 ectodomain is employed. Specifically, domains within the ectodomain that function as a CD72-ligand binding domain are employed. In certain embodiments, polypeptides contain a CD72-ligand binding domain that is an immunoglobulin variable region binding domain specific for a CD100 or CD5. In further embodiments, polypeptides contain a first immunoglobulin variable region binding domain specific for a CD100 or CD5 fused to a second immunoglobulin variable region binding domain specific for a different B-cell protein (e.g., FCRL1-6, CD19, CD20, CD22, CD32b, CD37, CD79a, CD79b, CD267 or CD269), wherein the first binding domain will have a lower affinity for CD100 or CD5 than the affinity of the second binding domain for the different B-cell protein.

Exemplary structures of such multi-specific fusion proteins, referred to herein as Xceptor molecules, include N-BD-ID-ED-C, N-ED-ID-BD-C, N-BD1-ID-BD2-C, wherein N— and —C refer to the amino- and carboxy terminus, respectively; BD is an immunoglobulin-like or immunoglobulin variable region binding domain; ID is an intervening domain; and ED is an extracellular or ectodomain, such as a receptor ligand binding domain, a cysteine rich domain (A domain; see WO 02/088171 and WO 04/044011), C-type lectin domain, semaphorin or semaphorin-like domain, or the like. In some constructs, the ID can comprise an immunoglobulin constant region or sub-region disposed between the first and second binding domains. In still further constructs, the BD and ED are each linked to the ID via the same or different linker (e.g., a linker comprising one to fifty amino acids) such as an immunoglobulin hinge region (made up of, for example, the upper and core regions) or functional variant thereof, or a lectin interdomain region or functional variant thereof, or a cluster of differentiation (CD) molecule stalk region or functional variant thereof.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, “about” or “consisting essentially of” mean±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include” and “comprise” are used synonymously. In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure.

A “binding domain” or “binding region” according to the present disclosure may be, for example, any protein, polypeptide, oligopeptide, or peptide that possesses the ability to specifically recognize and bind to a biological molecule (e.g., CD100 or other B-cell surface protein) or complex of more than one of the same or different molecule or assembly or aggregate, whether stable or transient (e.g., CD72/CD100 complex). Such biological molecules include proteins, polypeptides, oligopeptides, peptides, amino acids, or derivatives thereof, lipids, fatty acids, or derivatives thereof; carbohydrates, saccharides, or derivatives thereof; nucleotides, nucleosides, peptide nucleic acids, nucleic acid molecules, or derivatives thereof; glycoproteins, glycopeptides, glycolipids, lipoproteins, proteolipids, or derivatives thereof; other biological molecules that may be present in, for example, a biological sample; or any combination thereof. A binding region includes any naturally occurring, synthetic, semi-synthetic, or recombinantly produced binding partner for a biological molecule or other target of interest. A variety of assays are known for identifying binding domains of the present disclosure that specifically bind a particular target, including Western blot, ELISA, or Biacore analysis.

Binding domains and fusion proteins thereof of this disclosure can be capable of binding to a desired degree, including “specifically or selectively binding” a target while not significantly binding other components present in a test sample, if they bind a target molecule with an affinity or Kₐ (i.e., an equilibrium association constant of a particular binding interaction with units of 1/M) of, for example, greater than or equal to about 10⁵ M⁻¹, 10⁶ M⁻¹, 10² M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding domains refers to those binding domains with a Kₐ of at least 10⁷ M⁻¹, at least 10⁸ M⁻¹, at least 10⁹ M⁻¹, at least 10¹⁰ M⁻¹ at least 10¹¹ M⁻¹, at least 10¹² M⁻¹, at least 10¹³ M⁻¹, or greater. “Low affinity” binding domains refers to those binding domains with a Kₐ of up to 5×10⁷ M⁻¹, up to 10⁷ M⁻¹, up to 10⁶ M⁻¹, up to 10⁵ M⁻¹, or less. Alternatively, affinity may be defined as an equilibrium dissociation constant (K_{d}) of a particular binding interaction with units of M (e.g., 10⁻⁵ M to 10⁻¹³ M). Affinities of binding domain polypeptides and fusion proteins according to the present disclosure can be readily determined using conventional techniques (see, e.g., Scatchard et al. (1949) Ann. N.Y. Acad. Sci. 51:660; and U.S. Pat. Nos. 5,283,173; 5,468,614; Biacore® analysis; or the equivalent).

Binding domains of this disclosure can be generated as described herein or by a variety of methods known in the art (see, e.g., U.S. Pat. Nos. 6,291,161; 6,291,158). Sources include antibody gene sequences from various species (which can be formatted as antibodies, sFvs, scFvs or Fabs, such as in a phage library), including human, camelid (from camels, dromedaries, or llamas; Hamers-Casterman et al. (1993) Nature, 363:446 and Nguyen et al. (1998) J. Mol. Biol., 275:413), shark (Roux et al. (1998) Proc. Nat'l. Acad. Sci. (USA) 95:11804), fish (Nguyen et al. (2002) Immunogenetics, 54:39), rodent, avian, ovine, as well as sequences that encode random peptide libraries or sequences that encode an engineered diversity of amino acids in loop regions of alternative non-antibody scaffolds, such as fibrinogen domains (see, e.g., Weisel et al. (1985) Science 230:1388), Kunitz domains (see, e.g., U.S. Pat. No. 6,423,498), lipocalin domains (see, e.g., PCT Patent Application Publication No. WO 2006/095164), V-like domains (see, e.g., US Patent Application Publication No. 2007/0065431), C-type lectin domains (Zelensky and Gready (2005) FEBS J. 272:6179), mAb² or Fcab™ (see, e.g., PCT Patent Application Publication Nos. WO 2007/098934; WO 2006/072620), or the like. Additionally, traditional strategies for hybridoma development using, for example, a synthetic single chain CD100, CD5, FCRL1-6, CD19, CD20, CD22, CD32b, CD37, CD79a, CD79b, CD267 or CD269 as an immunogen in convenient systems (e.g., mice, HuMAb Mouse®, TC Mouse™, KM-Mouse®, llamas, chicken, rats, hamsters, rabbits, etc.) can be used to develop binding domains of this disclosure.

Terms understood by those in the art as referring to antibody technology are each given the meaning acquired in the art, unless expressly defined herein. For example, the terms “V_{L}” and “V_{H}” refer to the variable binding region derived from an antibody light and heavy chain, respectively. The variable binding regions are made up of discrete, well-defined sub-regions known as “complementarity determining regions” (CDRs) and “framework regions” (FRs). The terms “C_{L}” and “C_{H}” refer to an “immunoglobulin constant region,” i.e., a constant region derived from an antibody light or heavy chain, respectively, with the latter region understood to be further divisible into C_{H1}, C_{H2}, C_{H3} and C_{H4} constant region domains, depending on the antibody isotype (IgA, IgD, IgE, IgG, IgM) from which the region was derived. A portion of the constant region domains makes up the Fc region (the “fragment crystallizable” region), which contains domains responsible for the effector functions of an immunoglobulin, such as ADCC (antibody-dependent cell-mediated cytotoxicity), ADCP (antibody-dependent cell-mediated phagocytosis), CDC (complement-dependent cytotoxicity) and complement fixation, binding to Fc receptors, greater half-life in vivo relative to a polypeptide lacking an Fc region, protein A binding, and perhaps even placental transfer (see Capon et al. (1989) Nature, 337:525). Further, a polypeptide containing an Fc region allows for dimerization or multimerization of the polypeptide. A “hinge region,” also referred to herein as a “linker,” is an amino acid sequence interposed between and connecting the variable binding and constant regions of a single chain of an antibody, which is known in the art as providing flexibility in the form of a hinge to antibodies or antibody-like molecules.

The domain structure of immunoglobulins is amenable to engineering, in that the antigen binding domains and the domains conferring effector functions may be exchanged between immunoglobulin classes and subclasses Immunoglobulin structure and function are reviewed, for example, in Harlow et al., Eds., Antibodies: A Laboratory Manual, Chapter 14 (Cold Spring Harbor Laboratory, Cold Spring Harbor, 1988). An extensive introduction as well as detailed information about all aspects of recombinant antibody technology can be found in the textbook Recombinant Antibodies (John Wiley & Sons, NY, 1999). A comprehensive collection of detailed antibody engineering lab protocols can be found in R. Kontermann and S. Dübel, Eds., The Antibody Engineering Lab Manual (Springer Verlag, Heidelberg/New York, 2000).

“Derivative” as used herein refers to a chemically or biologically modified version of a compound that is structurally similar to a parent compound and (actually or theoretically) derivable from that parent compound. Generally, a “derivative” differs from an “analogue” in that a parent compound may be the starting material to generate a “derivative,” whereas the parent compound may not necessarily be used as the starting material to generate an “analogue.” An analogue may have different chemical or physical properties to the parent compound. For example, a derivative may be more hydrophilic or it may be a mutated sequence having altered reactivity (e.g., a CDR having an amino acid change that alters its affinity for a target) as compared to the parent compound or sequence.

The term “biological sample” includes a blood sample, biopsy specimen, tissue explant, organ culture, biological fluid or any other tissue or cell or other preparation from a subject or a biological source. A subject or biological source may, for example, be a human or non-human animal, a primary cell culture or culture adapted cell line including genetically engineered cell lines that may contain chromosomally integrated or episomal recombinant nucleic acid sequences, somatic cell hybrid cell lines, immortalized or immortalizable cell lines, differentiated or differentiatable cell lines, transformed cell lines, or the like. In further embodiments of this disclosure, a subject or biological source may be suspected of having or being at risk for having a disease, disorder or condition, including a malignant disease, disorder or condition or a B cell disorder. In certain embodiments, a subject or biological source may be suspected of having or being at risk for having a hyperproliferative, inflammatory, or autoimmune disease, and in certain other embodiments of this disclosure the subject or biological source may be known to be free of a risk or presence of such disease, disorder, or condition.

### CD72-Ligand Binding Domains

As set forth herein, CD72 comprises a type II transmembrane protein having an extracellular domain containing an extracellular C-type lectin-like domain and a cytoplasmic immunoreceptor tyrosine-based inhibitory motif (ITIM). A CD72-ligand binding domain of this disclosure can inhibit the inflammatory, autoimmune, or hyperproliferative activity associated with CD72. For example and not wishing to be bound by theory, a CD72-ligand binding domain can promote cell cycle arrest and apoptosis (see, e.g., Li et al. (2006) J. Immunol. 176:5321) and co-engagement of CD72-ligand (e.g., CD100) binding with other binding domains that impart, for example, their own death signal can more effectively kill malignant B cells. Various CD72-ligand binding domains are known in the art, including anti-CD100 antibodies, such as monoclonal antibodies BB18, BD16, NL014, NL026, NL037, NL056, NL057, NL008, NL010, NL126, NL128, NL153, or CDRs thereof (see, e.g., Herold et al. (1995) Int'l. Immunol. 7:1; Delaire et al. (1996) Tissue Antigen 48:456). Anti-CD100 antibodies, including monoclonal antibodies, can be prepared using techniques known in the art (see, e.g., US Patent Publication No. 2006/0233793). In another example, a CD72-ligand binding domain of this disclosure can comprise one or more CD100 binding domains present in a CD72 ectodomain.

CD72-ligand binding domains contemplated include a CD72 extracellular domain or sub-domain, a CD72 C-type lectin domain, or CD100-specific antibody-derived binding domain. In some embodiments, a CD72-ligand binding domain may be an extracellular domain (“ectodomain”) of a CD72, such as an extracellular portion containing a C-type lectin domain. As used herein, a CD72 ectodomain refers to a sCD72, an extracellular portion containing a C-type lectin domain, or any combination thereof. In certain embodiments, a CD72-ligand binding domain comprises a carboxy-terminal portion of CD72, such as the last 243 amino acids of CD72 as set forth in GenBank Accession No. NP_{—}001773.1 (SEQ ID NO:1). In other embodiments, a CD72-ligand binding domain comprises amino acids 200-359, 210-359, 221-359, or 233-359 of SEQ ID NO:1. In further embodiments, a CD72-ligand binding domain comprising amino acids 221-359 or 233-359 of SEQ ID NO:1 fused to an intervening domain via a linker that is a CD72 stalk region or a portion thereof, such as amino acids 117-232, 200-232, or 210-232 of SEQ ID NO:1.

In one aspect, a CD72-ligand binding domain or fusion protein thereof of this disclosure is specific for CD100 wherein it has an affinity with a dissociation constant (K_{d}) of about 10⁻⁵ M to less than about 10⁻⁸ M. In certain embodiments, the CD72-ligand binding domain or fusion protein thereof binds CD100 with an affinity of about 0.3 μM.

In an illustrative example, CD72-ligand binding domains of this disclosure specific for a CD100 molecule or other CD72 ligand (e.g., CD5) can be identified using a Fab phage library of fragments (see, e.g., Hoet et al. (2005) Nature Biotechnol. 23:344) by screening for binding to a synthetic or recombinant CD100 (using an amino acid sequence or fragment thereof as set forth in GenBank Accession No. NP_{—}006369.2) or other CD72 ligand. In certain embodiments, a CD100 molecule or other CD72 ligand (e.g., CD5) used to generate a CD72-ligand binding domain can further comprise an intervening domain or a dimerization domain, as described herein, such as an immunoglobulin Fc domain or fragment thereof.

In some embodiments, CD72-ligand binding domains of this disclosure comprise V_{H} and V_{L} domains as described herein. In certain embodiments, the V_{H} and V_{L} domains are rodent (e.g., mouse, rat), humanized, or human. In further embodiments, there are provided CD72-ligand binding domains of this disclosure that have a sequence that is at least 90%, at least 91%, at least 92%, at least 93%, at least 94%, at least 95%, at least 96%, at least 97%, at least 98%, at least 99%, at least 99.5%, or at least 100% identical to the amino acid sequence of one or more light chain variable regions (V_{L}) or to one or more heavy chain variable regions (V_{H}), or both, wherein each CDR have zero changes or no more than one, two, or three amino acid changes (i.e., many of the changes will be in the framework).

The terms “identical” or “percent identity,” in the context of two or more polypeptide or nucleic acid molecule sequences, means two or more sequences or subsequences that are the same or have a specified percentage of amino acid residues or nucleotides that are the same over a specified region (e.g., 60%, 65%, 70%, 75%, 80%, 85%, 90%, 91%, 92%, 93%, 94%, 95%, 96%, 97%, 98%, 99%, or 100% identity), when compared and aligned for maximum correspondence over a comparison window, or designated region, as measured using methods known in the art, such as a sequence comparison algorithm, by manual alignment, or by visual inspection. For example, preferred algorithms suitable for determining percent sequence identity and sequence similarity are the BLAST and BLAST 2.0 algorithms, which are described in Altschul et al. (1977) Nucleic Acids Res. 25:3389 and Altschul et al. (1990) J. Mol. Biol. 215:403, respectively.

In any of these or other embodiments described herein, the V_{L} and V_{H} domains may be arranged in either orientation and may be separated by about a five to about a 30 amino acid linker as disclosed herein or any other amino acid sequence capable of providing a spacer function compatible with interaction of the two sub-binding domains. In certain embodiments, a linker joining the V_{H} and V_{L} domains comprises an amino acid sequence as set forth in SEQ ID NO: 18-141, such as Linker 46 (SEQ ID NO:63), 130 (SEQ ID NO:138), or 131 (SEQ ID NO:139). Multi-specific binding domains will have at least two specific sub-binding domains, by analogy to camelid antibody organization, or at least four specific sub-binding domains, by analogy to the more conventional mammalian antibody organization of paired V_{H} and V_{L} chains.

In further embodiments, CD72-ligand binding domains and fusion proteins thereof of this disclosure may comprise a binding domain including one or more complementarity determining region (“CDR”), or multiple copies of one or more such CDRs, which have been obtained, derived, or designed from variable regions of an anti-CD100 or anti-CD5 scFv or Fab fragment or from heavy or light chain variable regions thereof. In certain embodiments, fusion proteins containing a first binding domain specific for CD100 or CD5 having such CDRs and a second binding domain specific for FCRL1, FCRL2, FCRL3, FCRL4, FCRL5, FCRL6, CD19, CD20, CD22, CD32b, CD37, CD79a, CD79b, CD267 or CD269 will have a first binding domain with an affinity for CD100 or CD5, respectively, that is less than (e.g., about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 50-fold, 100-fold, 1000-fold, or greater) the affinity the second binding domain has for FCRL1, FCRL2, FCRL3, FCRL4, FCRL5, FCRL6, CD19, CD20, CD22, CD32b, CD37, CD79a, CD79b, CD267 or CD269, respectively. For example, if the affinity of an anti-CD100 binding domain for CD100 is about 0.3 μM, then a B-cell protein binding domain having at least a 10-fold higher affinity for the B-cell protein (e.g., FCRL1-6, CD19, CD20, CD22, CD32b, CD37, CD79a, CD79b, CD267 or CD269) has a dissociation constant (K_{d}) of about 30 nM or less.

CDRs are defined in various ways in the art, including the Kabat, Chothia, AbM, and contact definitions. The Kabat definition is based on sequence variability and is the most commonly used definition to predict CDR regions (Johnson et al. (2000) Nucleic Acids Res. 28:214). The Chothia definition is based on the location of the structural loop regions (Chothia et al. (1986) J. Mol. Biol. 196:901; Chothia et al. (1989) Nature 342:877). The AbM definition, a compromise between the Kabat and Chothia definitions, is an integral suite of programs for antibody structure modeling produced by the Oxford Molecular Group (Martin et al. (1989) Proc. Nat'l. Acad. Sci. (USA) 86:9268; Rees et al., ABMTM, a computer program for modeling variable regions of antibodies, Oxford, UK; Oxford Molecular, Ltd.). An additional definition, known as the contact definition, has been recently introduced (see MacCallum et al. (1996) J. Mol. Biol. 5:732), which is based on analysis of available complex crystal structures.

By convention, the CDR domains in the heavy chain are referred to as H1, H2, and H3, which are numbered sequentially in order moving from the amino terminus to the carboxy terminus The CDR-H1 is about ten to 12 residues in length and starts four residues after a Cys according to the Chothia and AbM definitions, or five residues later according to the Kabat definition. The H1 can be followed by a Trp, Trp-Val, Trp-Ile, or Trp-Ala. The length of H1 is approximately ten to 12 residues according to the AbM definition, while the Chothia definition excludes the last four residues. The CDR-H2 starts 15 residues after the end of H1 according to the Kabat and AbM definitions, which is generally preceded by sequence Leu-Glu-Trp-Ile-Gly (but a number of variations are known) and is generally followed by sequence Lys/Arg-Leu/Ile/Val/Phe/Thr/Ala-Thr/Ser/Ile/Ala. According to the Kabat definition, the length of H2 is about 16 to 19 residues, while the AbM definition predicts the length to be nine to 12 residues. The CDR-H3 usually starts 33 residues after the end of H2, is generally preceded by the amino acid sequence Cys-Ala-Arg and followed by the amino acid Gly, and has a length that ranges from three to about 25 residues.

By convention, CDR regions in the light chain are referred to as L1, L2, and L3, which are numbered sequentially in order moving from the amino terminus to the carboxy terminus. The CDR-L1 (approximately ten to 17 residues in length) generally starts at about residue 24 and generally follows a Cys. The residue after the CDR-L1 is always Trp, which begins one of the following sequences: Trp-Tyr-Gln, Trp-Leu-Gln, Trp-Phe-Gln, or Trp-Tyr-Leu. The CDR-L2 (about seven residues in length) starts about 16 residues after the end of L1 and will generally follow residues Ile-Tyr, Val-Tyr, Ile-Lys, or Ile-Phe. The CDR-L3 usually starts 33 residues after the end of L2 and generally follows a Cys, which is generally followed by the sequence Phe-Gly-XXX-Gly and has a length of about seven to 11 residues.

Thus, a binding domain of this disclosure can comprise a single CDR from a variable region of an anti-CD100 or anti-CD5, or it can comprise multiple CDRs that can be the same or different. In certain embodiments, binding domains of this disclosure comprise V_{H} and V_{L} domains specific for a CD100 or CD5 comprising framework regions and CDR1, CDR2 and CDR3 regions, wherein (a) the V_{H} domain comprises an amino acid sequence of a heavy chain CDR3; or (b) the V_{L} domain comprises an amino acid sequence of a light chain CDR3; or (c) the binding domain comprises a V_{H} amino acid sequence of (a) and a V_{L} amino acid sequence of (b); or the binding domain comprises a V_{H} amino acid sequence of (a) and a V_{L} amino acid sequence of (b) and wherein the V_{H} and V_{L} are found in the same reference sequence. In further embodiments, binding domains of this disclosure comprise V_{H} and V_{L} domains specific for an CD100 or CD5 comprising framework regions and CDR1, CDR2 and CDR3 regions, wherein (a) the V_{H} domain comprises an amino acid sequence of a heavy chain CDR1, CDR2, and CDR3; or (b) the V_{L} domain comprises an amino acid sequence of a light chain CDR1, CDR2, and CDR3; or (c) the binding domain comprises a V_{H} amino acid sequence of (a) and a V_{L} amino acid sequence of (b); or the binding domain comprises a V_{H} amino acid sequence of (a) and a V_{L} amino acid sequence of (b), wherein the V_{H} and V_{L} amino acid sequences are from the same reference sequence.

In any of the embodiments described herein comprising specific CDRs, a binding domain can comprise (i) a V_{H} domain having an amino acid sequence that is at least 80%, 85%, 90%, 91%, 92%, 93%, 94%, 95%, 96%, 97%, 98%, or 99° A identical to the amino acid sequence of a V_{H} domain, wherein each CDR have zero changes or no more than one, two, or three amino acid changes (i.e., many of the changes will be in the framework); or (ii) a V_{L} domain having an amino acid sequence that is at least 80%, 85%, 90%, 91%, 92%, 93%, 94%, 95%, 96%, 97%, 98%, or 99% identical to the amino acid sequence of a V_{L} domain, wherein each CDR have zero changes or no more than one, two, or three amino acid changes (i.e., many of the changes will be in the framework); or (iii) both a V_{H} domain of (i) and a V_{L} domain of (ii); or both a V_{H} domain of (i) and a V_{L} domain of (ii) wherein the V_{H} and V_{L} are from the same reference sequence.

A CD72-ligand binding domain in fusion proteins of this disclosure may be an immunoglobulin-like domain, such as an immunoglobulin scaffold. Immunoglobulin scaffolds contemplated by this disclosure include a scFv, a domain antibody, or a heavy chain-only antibody. In a scFv, this disclosure contemplates the heavy and light chain variable regions are joined by any linker peptide described herein or known in the art to be compatible with domain or region joinder in a binding molecule. Exemplary linkers are linkers based on the Gly₄Ser linker motif, such as (Gly₄Ser)ₙ, wherein n=1-5. If a first domain of a fusion protein of this disclosure is based on a non-human immunoglobulin or includes non-human immunoglobulin CDRs, the binding domain may be “humanized” according to methods known in the art.

Alternatively, a CD72-ligand binding domain of fusion proteins of this disclosure may be a scaffold other than an immunoglobulin scaffold. Other scaffolds contemplated by this disclosure present the CD72 ligand-specific CDR(s) in a functional conformation. Other scaffolds contemplated include, but are not limited to an A domain molecule, a fibronectin III domain, an anticalin, an ankyrin-repeat engineered binding molecule, an adnectin, a Kunitz domain or a protein AZ domain affibody.

### B-Cell Specific Proteins

As noted above, the present disclosure provides polypeptides containing a B-cell binding domain, such as an immunoglobulin variable region or derivative thereof, such as an antibody, Fab, scFv, or the like, which is specific for a B-cell protein, such as a cell surface protein or receptor. In certain embodiments, the B cell protein is FCRL1, FCRL2, FCRL3, FCRL4, FCRL5, FCRL6, CD19, CD20, CD22, CD32b, CD37, CD79a, CD79b, CD267 or CD269. In further embodiments, a binding region or domain is a FCRL1, FCRL2, FCRL3, FCRL4, FCRL5, FCRL6, CD19, CD20, CD22, CD32b, CD37, CD79a, CD79b, CD267 or CD269 agonist (e.g. increases signaling or another biological activity) or antagonist (e.g., inhibits signaling or another biological activity). In certain embodiments, the present disclosure provides multi-specific fusion proteins containing a binding region or domain specific for a B-cell protein and a CD72-ligand binding domain wherein the B-cell specific binding domain has higher affinity for the targeted B-cell protein than the CD72-ligand binding domain has for the CD72 ligand, resulting in binding specificity to B-cells and specificity of action of the fusion proteins.

In certain embodiments, a multi-specific fusion protein contains a first and a second binding region or domain, wherein the first binding domain is a CD72-ligand binding domain having a dissociation constant (K_{d}) with a CD72 ligand that is 2-fold to 100-fold greater than the K_{d} of a second binding domain that is a B-cell protein (e.g., FCRL1, FCRL2, FCRL3, FCRL4, FCRL5, FCRL6, CD19, CD20, CD22, CD32b, CD37, CD79a, CD79b, CD267 or CD269) antagonist. In further embodiments, a multi-specific fusion protein contains a first binding domain that is a CD72-ligand binding domain having a dissociation constant (K_{d}) with a CD72 ligand of about 500 nM, and a second binding domain that is a B-cell protein agonist or antagonist having a K_{d} of about 10 nM or less with a B-cell protein, such as a FCRL1, FCRL2, FCRL3, FCRL4, FCRL5, FCRL6, CD19, CD20, CD22, CD32b, CD37, CD79a, CD79b, CD267 or CD269.

Another measure, the kinetic dissociation (k_{d}), also referred to herein as k_{OFF}, is a measure of the rate of complex dissociation and, thus, the ‘dwell time’ of the target molecule bound by a polypeptide binding domain of this disclosure. The k_{d} (k_{OFF}) has units of 1/sec. Exemplary B-cell specific protein binding domains of this disclosure can have a k_{OFF} of about 10⁴/sec (e.g., about a day) to about 10⁻⁸/sec or less. In certain embodiments, the k_{OFF} can range from about 10⁴/sec, about 10⁻²/sec, about 10⁻³/sec, about 10⁴/sec, about 10⁻⁵/sec, about 10⁻⁶/sec, about 10⁻⁷/sec, about 10⁻⁸/sec, about 10⁻⁹/sec, about 10⁻¹⁰/sec, or less (see Graff et al. (2004) Protein Eng. Des. Sel. 17:293). In some embodiments, a B-cell specific protein binding domain or fusion protein thereof of this disclosure will bind FCRL1, FCRL2, FCRL3, FCRL4, FCRL5, FCRL6, CD19, CD20, CD22, CD32b, CD37, CD79a, CD79b, CD267 or CD269 with higher affinity and have a lower k_{oFF} rate as compared to the cognate binding partner. In further embodiments, a B-cell specific protein binding domain or fusion protein thereof of this disclosure that blocks or alters FCRL1, FCRL2, FCRL3, FCRL4, FCRL5, FCRL6, CD19, CD20, CD22, CD32b, CD37, CD79a CD79b, CD267 or CD269 cell surface activity may have a more moderate affinity (i.e., a K_{d} of about 10⁻⁸ M to about 10⁻⁹ M) and a more moderate off rate (i.e., a k_{OFF} closer to about 10⁴/sec) as compared to the affinity and dimerization rate of a cognate partner.

In certain embodiments, a binding domain of this disclosure may be an immunoglobulin-like domain, such as an immunoglobulin scaffold. Immunoglobulin scaffolds contemplated in this disclosure include a scFv, Fab, a domain antibody, or a heavy chain-only antibody. In further embodiments, there are provided anti-B-cell protein antibodies (e.g., non-human such as mouse or rat, chimeric, humanized, human) or Fab fragments or scFv fragments that have an amino acid sequence that is at least 80%, 85%, 90%, 91%, 92%, 93%, 94%, 95%, 96%, 97%, 98%, 99%, or 100% identical to the amino acid sequence of a selected V_{H} and V_{L} domain, wherein each CDR can have zero changes or no more than one, two, or three amino acid changes (i.e., many of the changes will be in the framework). Alternatively, binding domains of this disclosure may be part of a scaffold other than an immunoglobulin. Other scaffolds contemplated include an A domain molecule, a fibronectin III domain, an anticalin, an ankyrin-repeat engineered binding molecule, an adnectin, a Kunitz domain, or a protein AZ domain affibody.

### CD19 Binding Domains

As noted above, in certain embodiments the present disclosure provides polypeptides containing a binding region or domain that is specific for CD19. In certain embodiments, such binding domains are CD19 agonists or antagonists. Exemplary binding domains specific for a CD19 include immunoglobulin variable binding domains or derivatives thereof (e.g., an antibody, Fab, scFv, or the like).

CD19 is a cell surface molecule expressed only by B lymphocytes and follicular dendritic cells of the hematopoietic system. It is the earliest of the B-lineage-restricted antigens to be expressed and is present on most pre-B cells, most non-T-cell acute lymphocytic leukemia cells and B-cell type chronic lymphocytic leukemia cells. CD19 is involved in B cell signaling pathways, and is thought to enhance antigen stimulation of the B cell receptor, which is made up of surface immunoglobulin (sIg) and a CD79a/Cd79b heterodimer. For example, coligation of CD19 with the antigen receptor of B cells decreases the threshold for antigen receptor-dependent stimulation by two orders of magnitude (Carter et al. (1992) Science 256:105). CD19 has also been shown to form a complex with CD21, CD81 and CD225 in the membrane of mature B cells.

CD19 is a 556 amino acid cell surface protein (Genbank Accession No. NP_{—}001761, SwissProt Entry P15391) comprising a signal sequence and a putative extracellular region containing two immunoglobulin-like domains, an immunoglobulin-like C2-type 1 domain from residues 20 to 113, and an immunoglobulin-like C2-type 2 domain from residues 176 to 277. CD19 also contains an approximately 240-amino acid cytoplasmic tail with nine conserved tyrosine and serine residues. The tyrosine and serine residues are phosphorylated by various kinases involved in B cell signaling, implicating CD19 in many signaling pathways in the B cell. It is believed that CD19 functions as an adaptor protein for the amplification of Src family kinases that are important for intrinsic and antigen receptor-induced signal transduction (Fujimoto et al. (2000) Immunity 13:47).

In some embodiments, binding domains of this disclosure comprise V_{L} and V_{H} domains specific for a CD19 as described herein. In certain embodiments, the V_{L} and V_{H} domains are human. An exemplary binding domain containing such V_{L} and V_{H} domains specific for CD19 is set forth in SEQ ID NO: 9, with amino acids 21-132 and 148-271 representing the V_{L} and V_{H} domains, respectively. In further embodiments, there are provided polypeptide binding domains specific for a CD19 comprising a sequence that is at least 90%, at least 91%, at least 92%, at least 93%, at least 94%, at least 95%, at least 96%, at least 97%, at least 98%, at least 99%, at least 99.5%, or at least 100% identical to amino acids 21-132 of a light chain variable region (V_{L}) or to amino acids 148-271 of a heavy chain variable region (V_{H}), or both, as set forth in SEQ ID NO:9, wherein each CDR can have zero changes or no more than one, two, or three amino acid changes (i.e., many of the changes will be in the framework).

In any of these or other embodiments described herein, the V_{L} and V_{H} domains may be arranged in either orientation and may be separated by up to about a 30 amino acid linker as disclosed herein or any other amino acid sequence capable of providing a spacer function compatible with interaction of the two sub-binding domains. In certain embodiments, a linker joining the V_{L} and V_{H} domains comprises an amino acid sequence as set forth in SEQ ID NO: 18-141, such as Linker 46 (SEQ ID NO:63), 130 (SEQ ID NO:138), or 131 (SEQ ID NO:139). Multi-specific binding domains can have at least two specific sub-binding domains, by analogy to camelid antibody organization, or at least four specific sub-binding domains, by analogy to the more conventional mammalian antibody organization of paired V_{L} and V_{H} chains.

In further embodiments, binding domains specific for CD19 of this disclosure may comprise one or more complementarity determining region (“CDR”), or multiple copies of one or more such CDRs, which have been obtained, derived, or designed from variable regions of an anti-CD19 scFv or Fab fragment or from heavy or light chain variable regions thereof. Thus, a binding domain of this disclosure can comprise a single CDR from a variable region of an anti-CD19, or it can comprise multiple CDRs that can be the same or different. In certain embodiments, binding domains of this disclosure comprise V_{L} and V_{H} domains specific for a CD19 comprising framework regions and CDR1, CDR2 and CDR3 regions, wherein (a) the V_{H} domain comprises the amino acid sequence of a heavy chain CDR3 found in SEQ ID NO:9; or (b) the V_{L} domain comprises the amino acid sequence of a light chain CDR3 found in SEQ ID NO:9; or (c) the binding domain comprises a V_{H} amino acid sequence of (a) and a V_{L} amino acid sequence of (b). In any of the embodiments described herein comprising specific CDRs against CD19, a binding domain can comprise (i) a V_{H} domain having an amino acid sequence that is at least 80%, 85%, 90%, 91%, 92%, 93%, 94%, 95%, 96%, 97%, 98% or 99% identical to the amino acid sequence of a V_{H} domain found in SEQ ID NO:9, wherein each CDR can have zero changes or no more than one, two, or three amino acid changes (i.e., many of the changes will be in the framework); or (ii) a V_{L} domain having an amino acid sequence that is at least 80%, 85%, 90%, 91%, 92%, 93%, 94%, 95%, 96%, 97%, 98%, or 99% identical to the amino acid sequence of a V_{L} domain found in SEQ ID NO:9, wherein each CDR can have zero changes or no more than one, two, or three amino acid changes (i.e., many of the changes will be in the framework); or (iii) both a V_{H} domain of (i) and a V_{L} domain of (ii).

### CD37 Binding Domains

As noted above, in certain embodiments the present disclosure provides polypeptides containing a binding region or domain that is specific for CD37. In certain embodiments, such binding domains are CD37 agonists (i.e., can increase CD37 signaling) or CD37 antagonists (i.e., decrease CD37 activity). Exemplary binding domains specific for a CD37 include immunoglobulin variable binding domains or derivatives thereof (e.g., an antibody, Fab, scFv, or the like).

CD37 is a heavily glycosylated 40-52 kDa protein that is B-cell lineage-specific cell surface molecule and belongs to the tetraspanin transmembrane family of cell surface antigens. It traverses the cell membrane four times forming two extracellular loops and exposing its amino and carboxy ends to the cytoplasm. CD37 is highly expressed on normal antibody-producing B-cells, but is not expressed on pre-B-cells or plasma cells. The expression of CD37 on resting and activated T cells, monocytes and granulocytes is low and there is no detectable CD37 expression on NK cells, platelets or erythrocytes (see Belov et al. (2001) Cancer Res. 61:4483; Schwartz-Albiez et al. (1988) J. Immunol. 140:905; and Link et al. (1988) J. Immunol. 137:3013). Aside from normal B-cells, almost all B-cell malignancies are positive for CD37 expression, including CLL, NHL, and hairy cell leukemia (Moore et al. (1987) J. Pathol. 152:13; Merson and Brochier (1988) Immunol. Lett. 19:269; and Faure et al. (1990) Am. J. Dermatopathol. 12:122). Mice lacking CD37 have low levels of serum IgG1 and are impaired in their humoral response to viral antigens, indicating that CD37 participates in the regulation of B-cell function. CD37 appears to act as a non-classical, co-stimulatory molecule or by directly influencing antigen presentation via complex formation with MHC class II molecules (see Knobeloch et al. (2000) Mol. Cell. Biol. 20:5363). CD37 also may play a role in TCR signaling (see Van Spriel et al. (2004) J. Immunol. 172:2953).

In some embodiments, binding domains of this disclosure comprise V_{L} and V_{H} domains specific for a CD37 as described herein. In certain embodiments, the V_{L} and V_{H} domains are human. An exemplary binding domain containing such V_{L} and V_{H} domains specific for CD37 is set forth in SEQ ID NO: 11, with amino acids 162-268 and 21-135 representing the V_{L} and V_{H} domains, respectively. In further embodiments, there are provided polypeptide binding domains specific for a CD19 comprising a sequence that is at least 90%, at least 91%, at least 92%, at least 93%, at least 94%, at least 95%, at least 96%, at least 97%, at least 98%, at least 99%, at least 99.5%, or at least 100% identical to amino acids 162-268 of a light chain variable region (V_{L}) or to amino acids 21-135 of a heavy chain variable region (V_{H}), or both, as set forth in SEQ ID NO:11, wherein each CDR can have zero changes or no more than one, two, or three amino acid changes (i.e., many of the changes will be in the framework). Other exemplary CD37 antagonists (e.g., V_{L} and V_{H} domains specific for a CD37) useful in the fusion proteins of this disclosure are described in US Patent Application Publication Nos. 2007/0059306 and 2008/0279850.

In any of these or other embodiments described herein, the V_{L} and V_{H} domains may be arranged in either orientation and may be separated by up to about a 30 amino acid linker as disclosed herein or any other amino acid sequence capable of providing a spacer function compatible with interaction of the two sub-binding domains. In certain embodiments, a linker joining the V_{L} and V_{H} domains comprises an amino acid sequence as set forth in SEQ ID NO: 18-141, such as Linker 46 (SEQ ID NO:63), 130 (SEQ ID NO:138), or 131 (SEQ ID NO:139). Multi-specific binding domains can have at least two specific sub-binding domains, by analogy to camelid antibody organization, or at least four specific sub-binding domains, by analogy to the more conventional mammalian antibody organization of paired V_{L} and V_{H} chains.

In further embodiments, binding domains specific for CD37 of this disclosure may comprise one or more complementarity determining region (“CDR”), or multiple copies of one or more such CDRs, which have been obtained, derived, or designed from variable regions of an anti-CD37 scFv or Fab fragment or from heavy or light chain variable regions thereof. Thus, a binding domain of this disclosure can comprise a single CDR from a variable region of an anti-CD37, or it can comprise multiple CDRs that can be the same or different. In certain embodiments, binding domains of this disclosure comprise V_{L} and V_{H} domains specific for a CD37 comprising framework regions and CDR1, CDR2 and CDR3 regions, wherein (a) the V_{H} domain comprises the amino acid sequence of a heavy chain CDR3 found in SEQ ID NO:11; or (b) the V_{L} domain comprises the amino acid sequence of a light chain CDR3 found in SEQ ID NO:11; or (c) the binding domain comprises a V_{H} amino acid sequence of (a) and a V_{L} amino acid sequence of (b). In any of the embodiments described herein comprising specific CDRs against CD37, a binding domain can comprise (i) a V_{H} domain having an amino acid sequence that is at least 80%, 85%, 90%, 91%, 92%, 93%, 94%, 95%, 96%, 97%, 98%, or 99% identical to the amino acid sequence of a V_{H} domain found in SEQ ID NO:11, wherein each CDR can have zero changes or no more than one, two, or three amino acid changes (i.e., many of the changes will be in the framework); or (ii) a V_{L} domain having an amino acid sequence that is at least 80%, 85%, 90%, 91%, 92%, 93%, 94%, 95%, 96%, 97%, 98%, or 99% identical to the amino acid sequence of a V_{L} domain found in SEQ ID NO:11, wherein each CDR can have zero changes or no more than one, two, or three amino acid changes (i.e., many of the changes will be in the framework); or (iii) both a V_{H} domain of (i) and a V_{L} domain of (ii).

### CD79 Binding Domains

As noted above, in certain embodiments the present disclosure provides polypeptides containing a binding region or domain that is specific for a CD79a or CD79b. In certain embodiments, such binding domains are CD79a or CD79b agonists or antagonists. Exemplary binding domains specific for a CD79a or CD79b include immunoglobulin variable binding domains or derivatives thereof (e.g., an antibody, Fab, scFv, or the like).

B-cell antigen receptor (BCR) is a multimeric complex that includes the antigen-specific component referred to as a surface immunoglobulin (sIg). The sIg associates non-covalently with two other proteins, Ig-α (CD79a) and Ig-β (CD79b), which are necessary for expression and function of the BCR complex. CD79a and CD79b, as a heterodimer, comprise a key component of the BCR involved in regulating B cell development and activity in vivo (Weinands et al. (2001) Int. Rev. Immunol. 20:679). CD79 (a and b) is expressed almost exclusively on B cells, including memory B cells and B cell neoplasms, and CD79a and CD79b expression precedes immunoglobulin heavy-chain gene rearrangement and CD20 expression during B-cell development (Chu et al. (2001) Appl. Immunohistochem. Mol. Morphol. 9:97). Signaling through the BCR complex is also required to prevent apoptosis of resting B cells (Kraus et al. (2004) Cell 117:787).

CD79a is expressed as two different isoforms (CD79a isoform 1 precursor, GenBank Accession No. NP_{—}001774, 226 amino acids, and CD79a isoform 2 precursor, GenBank Accession No. NP_{—}067612, 188 amino acids). Additional splice variants have also been identified from various cDNA libraries. CD79a is a single-pass type I membrane protein. Analysis of the CD79a isoform 1 precursor protein shows a 32 amino acid signal sequence, a 111 amino acid extracellular domain and a 61 amino acid cytoplasmic domain (Swiss-Prot entry P11912). The extracellular domain comprises an immunoglobulin C2-like region from approximately residues 33 to 116. The cytoplasmic domain of Ig-α contains several conserved regions and phosphorylation sites. For example, the cytoplasmic region comprises an immunoreceptor tyrosine-based activation motif (ITAM) from approximately residues 177 to 205, and several additional tyrosine, serine and threonine phosphorylation sites.

CD79b is expressed as three different isoforms (CD79b isoform 1 precursor, GenBank Accession No. NP_{—}000617., 229 amino acids, and CD79b isoform 2 precursor, GenBank Accession No. NP_{—}067613., 125 amino acids, CD79b isoform 3 precursor, GenBank Accession No. NP_{—}001035022, 230 amino acids). CD79b is a single-pass type I membrane protein. Based on the 229 amino acid precursor protein sequence, CD79b comprises a 28 amino acid signal sequence, a 131 amino acid extracellular domain and 49 amino acid cytoplasmic domain (Swiss-Prot entry P40259). The extracellular domain comprises an immunoglobulin V-like region from approximately residues 38 to 138. The cytoplasmic domain of Ig-β contains several conserved regions and phosphorylation sites. For example, the cytoplasmic region comprises an ITAM from approximately residues 185 to 213.

Upon B cell receptor binding, CD79a and CD79b become phosphorylated on tyrosine residues of the ITAM region, as well as at serine and threonine residues on CD79a. CD79b enhances phosphorylation of CD79a, possibly by recruiting kinases which phosphorylate CD79a or by recruiting proteins which bind to CD79a and protect it from dephosphorylation. Active CD79a, in turn, stimulates downstream signaling pathways involved in BCR signaling, including SYK tyrosine kinase autophosphorylation and activation and BLNK/SLP65 tyrosine kinase, bringing BLNK into proximity with SYK and allowing SYK to phosphorylate BLNK. Studies have indicated that the serine/threonine residues in the CD79a tail negatively regulate ITAM phosphorylation and other downstream signaling (Muller et al. (2000) Proc. Nat'l. Acad. Sci. USA 97:8451). CD79a also interacts with and increases activity of some Src-family tyrosine kinases and represses BCR signaling during development of immature B cells.

In some embodiments, binding domains of this disclosure comprise V_{L} and V_{H} domains specific for a CD79a or CD79b as described herein. In certain embodiments, there are provided polypeptide binding domains specific for a CD79a or CD79b comprising a sequence that is at least 90%, at least 91%, at least 92%, at least 93%, at least 94%, at least 95%, at least 96%, at least 97%, at least 98%, at least 99%, at least 99.5%, or at least 100% identical to a light chain variable region (V_{L}) or to a heavy chain variable region (V_{H}), or both, wherein each CDR can have zero changes or no more than one, two, or three amino acid changes (i.e., many of the changes will be in the framework), from a human anti-CD79a or anti-CD79b antibody, respectively.

In any of these or other embodiments described herein, the V_{L} and V_{H} domains may be arranged in either orientation and may be separated by up to about a 30 amino acid linker as disclosed herein or any other amino acid sequence capable of providing a spacer function compatible with interaction of the two sub-binding domains. In certain embodiments, a linker joining the V_{L} and V_{H} domains comprises an amino acid sequence as set forth in SEQ ID NO: 18-141, such as Linker 46 (SEQ ID NO:63), 130 (SEQ ID NO:138), or 131 (SEQ ID NO:139). Multi-specific binding domains can have at least two specific sub-binding domains, by analogy to camelid antibody organization, or at least four specific sub-binding domains, by analogy to the more conventional mammalian antibody organization of paired V_{L} and V_{H} chains.

In further embodiments, binding domains specific for CD79a or CD79b of this disclosure may comprise one or more complementarity determining region (“CDR”), or multiple copies of one or more such CDRs, which have been obtained, derived, or designed from variable regions of an anti-CD79a or anti-CD79b scFv or Fab fragment or from heavy or light chain variable regions thereof. Thus, a binding domain of this disclosure can comprise a single CDR from a variable region of an anti-CD79a or anti-CD79b, or it can comprise multiple CDRs that can be the same or different. In certain embodiments, binding domains of this disclosure comprise V_{L} and V_{H} domains specific for a CD79a or CD79b comprising framework regions and CDR1, CDR2 and CDR3 regions, wherein (a) the V_{H} domain comprises the amino acid sequence of a heavy chain CDR3; or (b) the V_{L} domain comprises the amino acid sequence of a light chain CDR3; or (c) the binding domain comprises a V_{H} amino acid sequence of (a) and a V_{L} amino acid sequence of (b). In any of the embodiments described herein comprising specific CDRs against CD79a or CD79b, a binding domain can comprise (i) a V_{H} domain having an amino acid sequence that is at least 80%, 85%, 90%, 91%, 92%, 93%, 94%, 95%, 96%, 97%, 98%, or 99% identical to the amino acid sequence of a V_{H} domain, wherein each CDR can have zero changes or no more than one, two, or three amino acid changes (i.e., many of the changes will be in the framework); or (ii) a V_{L} domain having an amino acid sequence that is at least 80%, 85%, 90%, 91%, 92%, 93%, 94%, 95%, 96%, 97%, 98%, or 99% identical to the amino acid sequence of a V_{L} domain, wherein each CDR can have zero changes or no more than one, two, or three amino acid changes (i.e., many of the changes will be in the framework); or (iii) both a V_{H} domain of (i) and a V_{L} domain of (ii).

### FCRL Binding Domains

As noted above, in certain embodiments the present disclosure provides polypeptides containing a binding region or domain that is specific for a Fc receptor-like protein 1 (FCRL1), FCRL2, FCRL3, FCRL4, FCRL5, or FCRL6. In certain embodiments, such binding domains are FCRL1, FCRL2, FCRL3, FCRL4, FCRL5 or FCRL6 agonists (i.e., can increase FCRL signaling or other biological activity, also known as Ig superfamily receptor translocation-associated gene or IRTA) or antagonists (i.e., can increase FCRL signaling or other biological activity). Exemplary binding domains specific for any one of FCRL1-6, respectively, include, for example, immunoglobulin variable binding domains or derivatives thereof (e.g., an antibody, Fab, scFv, or the like).

FCRL1 is expressed as a 429 amino acid protein (GenBank Accession No. NP_{—}443170.1), FCRL2 as a 508 and 255 amino acid protein (GenBank Accession No. NP_{—}110391.2 isoform b and NP_{—}620075.1 isoform a, respectively), FCRL3 as a 732 amino acid protein (GenBank Accession No. NP_{—}443171.2), FCRL4 as a 515 amino acid protein (GenBank Accession No. NP_{—}112572.1), FCRL5 as a 977 amino acid protein (GenBank Accession No. NP_{—}112571.1), and FCRL6 as a 434 amino acid protein (GenBank Accession No. NP_{—}001004310.2). All FCRL proteins are transmembrane receptors closely related to Fc receptors in their most amino-terminal extracellular domains and contain ITIM and ITAM-like domains on the cytoplasmic domain. The FCRL probably have a role in normal B-cell activation and possibly in the development of neoplasia (see Miller et al. (2002) Blood 99:2662).

In some embodiments, binding domains of this disclosure comprise V_{L} and V_{H} domains specific for any one of FCRL1-6 as described herein. In certain embodiments, there are provided polypeptide binding domains specific for any one of FCRL1-6 comprising a sequence that is at least 90%, at least 91%, at least 92%, at least 93%, at least 94%, at least 95%, at least 96%, at least 97%, at least 98%, at least 99%, at least 99.5%, or at least 100% identical to a light chain variable region (V_{L}) or to a heavy chain variable region (V_{H}), or both, wherein each CDR can have zero changes or no more than one, two, or three amino acid changes (i.e., many of the changes will be in the framework), from a human anti-FCRL1, 2, 3, 4, 5, or 6, respectively.

In any of these or other embodiments described herein, the V_{L} and V_{H} domains may be arranged in either orientation and may be separated by up to about a 30 amino acid linker as disclosed herein or any other amino acid sequence capable of providing a spacer function compatible with interaction of the two sub-binding domains. In certain embodiments, a linker joining the V_{L} and V_{H} domains comprises an amino acid sequence as set forth in SEQ ID NO: 18-141, such as Linker 46 (SEQ ID NO:63), 130 (SEQ ID NO:138), or 131 (SEQ ID NO:139). Multi-specific binding domains can have at least two specific sub-binding domains, by analogy to camelid antibody organization, or at least four specific sub-binding domains, by analogy to the more conventional mammalian antibody organization of paired V_{L} and V_{H} chains.

In further embodiments, binding domains specific for any one of FCRL1-6 of this disclosure may comprise one or more complementarity determining region (“CDR”), or multiple copies of one or more such CDRs, which have been obtained, derived, or designed from variable regions of anti-FCRL1, 2, 3, 4, 5, or 6, respectively, scFv or Fab fragment or from heavy or light chain variable regions thereof. Thus, a binding domain of this disclosure can comprise a single CDR from a variable region of anti-FCRL1, 2, 3, 4, 5, or 6, respectively, or it can comprise multiple CDRs that can be the same or different. In certain embodiments, binding domains of this disclosure comprise V_{L} and V_{H} domains specific for any one of FCRL1-6 comprising framework regions and CDR1, CDR2 and CDR3 regions, wherein (a) the V_{H} domain comprises the amino acid sequence of a heavy chain CDR3; or (b) the V_{L} domain comprises the amino acid sequence of a light chain CDR3; or (c) the binding domain comprises a V_{H} amino acid sequence of (a) and a V_{L} amino acid sequence of (b). In any of the embodiments described herein comprising specific CDRs against any one of FCRL1, 2, 3, 4, 5, or 6, respectively, a binding domain can comprise (i) a V_{H} domain having an amino acid sequence that is at least 80%, 85%, 90%, 91%, 92%, 93%, 94%, 95%, 96%, 97%, 98%, or 99% identical to the amino acid sequence of a V_{H} domain, wherein each CDR can have zero changes or no more than one, two, or three amino acid changes (i.e., many of the changes will be in the framework); or (ii) a V_{L} domain having an amino acid sequence that is at least 80%, 85%, 90%, 91%, 92%, 93%, 94%, 95%, 96%, 97%, 98%, or 99% identical to the amino acid sequence of a V_{L} domain, wherein each CDR can have zero changes or no more than one, two, or three amino acid changes (i.e., many of the changes will be in the framework); or (iii) both a V_{H} domain of (i) and a V_{L} domain of (ii).

### CD20 Binding Domains

As noted above, in certain embodiments the present disclosure provides polypeptides containing a binding region or domain that is a CD20 antagonist (i.e., can inhibit CD20 signaling) or agonist. Exemplary CD20 antagonists and agonists include binding domains specific for a CD20, such as an immunoglobulin variable binding domain or derivative thereof (e.g., an antibody, Fab, scFv, or the like).

CD20 was the first human B-cell lineage-specific surface molecule identified by a monoclonal antibody. It is a non-glycosylated, hydrophobic 35 kDa B-cell transmembrane phosphoprotein that has both its amino and carboxy ends situated inside the cell (Einfeld et al., EMBO J. 1988, 7:711-17). CD20 is expressed by all normal mature B-cells, but is not expressed by precursor B-cells or plasma cells. Natural ligands for CD20 have not been identified, and the function of CD20 in B-cell biology is still incompletely understood. Anti-CD20 monoclonal antibodies affect the viability and growth of B-cells. (Clark et al., Proc. Natl. Acad. Sci. USA 1986, 83:4494-98). Extensive cross-linking of CD20 can induce apoptosis in B lymphoma cell lines (Shan et al., Blood 1998, 91:1644-52), and cross-linking of CD20 on the cell surface has been reported to increase the magnitude and enhance the kinetics of signal transduction (Deans et al., J. Immunol. 1993, 146:846-53. The presence of multiple membrane spanning domains in the CD20 polypeptide (Einfeld et al., EMBO J. 1988, 7:711-17; Stamenkovic et al., J. Exp. Med. 1988, 167:1975-80; Tedder et al., J. Immunol. 1988, 141:4388-4394), prevent CD20 internalization after antibody binding, and this was recognized as an important feature for therapy of B-cell malignancies when a murine CD20 monoclonal antibody, 1F5, was injected into patients with B-cell lymphoma, resulting in significant depletion of malignant cells and partial clinical responses (Press et al., Blood 1987, 69:584-91).

In some embodiments, binding domains of this disclosure comprise V_{L} and V_{H} domains specific for a CD20 as described herein In certain embodiments, there are provided polypeptide binding domains specific for a CD20 comprising a sequence that is at least 90%, at least 91%, at least 92%, at least 93%, at least 94%, at least 95%, at least 96%, at least 97%, at least 98%, at least 99%, at least 99.5%, or at least 100% identical to a light chain variable region (V_{L}) or to a heavy chain variable region (V_{H}), or both, wherein each CDR can have zero changes or no more than one, two, or three amino acid changes (i.e., many of the changes will be in the framework), from an anti-CD20 scFv as disclosed in US Patent Application Publication No. 2007/0237779. In further embodiments, there are provided polypeptide binding domains specific for a CD20 comprising a sequence that is at least 90%, at least 91%, at least 92%, at least 93%, at least 94%, at least 95%, at least 96%, at least 97%, at least 98%, at least 99%, at least 99.5%, or at least 100% identical to a light chain variable region (V_{L}) or to a heavy chain variable region (V_{H}), or both, wherein each CDR can have zero changes or no more than one, two, or three amino acid changes (i.e., many of the changes will be in the framework), such as a humanized anti-CD20 as disclosed in PCT Publication No. WO 2008/156713 or US Patent Application Publication No. 2006/0024300.

In any of these or other embodiments described herein, the V_{L} and V_{H} domains may be arranged in either orientation and may be separated by up to about a 30 amino acid linker as disclosed herein or any other amino acid sequence capable of providing a spacer function compatible with interaction of the two sub-binding domains. In certain embodiments, a linker joining the V_{L} and V_{H} domains comprises an amino acid sequence as set forth in SEQ ID NO: 18-141, such as Linker 46 (SEQ ID NO:63), 130 (SEQ ID NO:138), or 131 (SEQ ID NO:139). Multi-specific binding domains can have at least two specific sub-binding domains, by analogy to camelid antibody organization, or at least four specific sub-binding domains, by analogy to the more conventional mammalian antibody organization of paired V_{L} and V_{H} chains.

In further embodiments, binding domains specific for a CD20 of this disclosure may comprise one or more complementarity determining region (“CDR”), or multiple copies of one or more such CDRs, which have been obtained, derived, or designed from variable regions of anti-CD20 disclosed in PCT Publication No. WO 2008/156713 or US Patent Application Publication No. 2006/0024300, scFv or Fab fragment or from heavy or light chain variable regions thereof. Thus, a binding domain of this disclosure can comprise a single CDR from a variable region of anti-CD20 disclosed in PCT Publication No. WO 2008/156713 or US Patent Application Publication No. 2006/0024300, or it can comprise multiple CDRs that can be the same or different. In certain embodiments, binding domains of this disclosure comprise V_{L} and V_{H} domains specific for a CD20 comprising framework regions and CDR1, CDR2 and CDR3 regions, wherein (a) the V_{H} domain comprises the amino acid sequence of a heavy chain CDR3 disclosed in PCT Publication No. WO 2008/156713 or US Patent Application Publication No. 2006/0024300; or (b) the V_{L} domain comprises the amino acid sequence of a light chain CDR3 disclosed in PCT Publication No. WO 2008/156713 or US Patent Application Publication No. 2006/0024300; or (c) the binding domain comprises a V_{H} amino acid sequence of (a) and a V_{L} amino acid sequence of (b). In any of the embodiments described herein comprising specific CDRs against a CD20, a binding domain can comprise (i) a V_{H} domain having an amino acid sequence that is at least 80%, 85%, 90%, 91%, 92%, 93%, 94%, 95%, 96%, 97%, 98%, or 99% identical to the amino acid sequence of a V_{H} domain disclosed in US Patent Application Publication No. 2006/0024300, wherein each CDR can have zero changes or no more than one, two, or three amino acid changes (i.e., many of the changes will be in the framework); or (ii) a V_{L} domain having an amino acid sequence that is at least 80%, 85%, 90%, 91%, 92%, 93%, 94%, 95%, 96%, 97%, 98%, or 99% identical to the amino acid sequence of a V_{L} domain disclosed in US Patent Application Publication No. 2006/0024300, wherein each CDR can have zero changes or no more than one, two, or three amino acid changes (i.e., many of the changes will be in the framework); or (iii) both a V_{H} domain of (i) and a V_{L} domain of (ii).

### CD22 Binding Domains

As noted above, in certain embodiments the present disclosure provides polypeptides containing a binding region or domain that is specific for CD22. In certain embodiments, the binding domain is a CD22 antagonist (i.e., can inhibit CD22 signaling) or agonist. Exemplary CD22 antagonists or agonists include binding domains specific for a CD22, such as an immunoglobulin variable binding domain or derivative thereof (e.g., an antibody, Fab, scFv, or the like).

The human B-lymphocyte-restricted antigen CD22 is expressed as an 847 amino acid protein (GenBank Accession No. NP_{—}001762.2) early in B-cell development in pro-B cells, as a cytoplasmic protein, and later in B-cell development, at the late pre-B-cell stage, as a cell surface protein. Once expressed as a membrane protein, CD22 persists on B cells until they differentiate into plasma cells. The presence of cytoplasmic CD22 is a useful marker for B-cell precursor acute lymphocytic leukemia. CD22 appears to be a heterodimer consisting of 130- and 140-kD glycoproteins with protein cores of 80 and 100 kD, respectively. Studies of the structure of the two proteins indicate that the larger subunit has an extracellular portion of seven immunoglobulin domains, one V-like, and six C-like, and a smaller subunit of five Ig domains, one V-like and four C-like domains. The CD22 polypeptide is structurally related to myelin-associated glycoprotein (MAG), neural cell adhesion molecule (NCAM), and carcinoembryonic antigen (CEA). Consistent with the structural similarities to the adhesion molecules, CD22 participates in adhesion between B cells and other cell types (see Wilson et al. (1991) J. Exp. Med. 173:137).

In some embodiments, binding domains of this disclosure comprise V_{L} and V_{H} domains specific for a CD22 as described herein. In certain embodiments, there are provided polypeptide binding domains specific for a CD22 comprising a sequence that is at least 90%, at least 91%, at least 92%, at least 93%, at least 94%, at least 95%, at least 96%, at least 97%, at least 98%, at least 99%, at least 99.5%, or at least 100% identical to a light chain variable region (V_{L}) or to a heavy chain variable region (V_{H}), or both, wherein each CDR can have zero changes or no more than one, two, or three amino acid changes (i.e., many of the changes will be in the framework), from a human anti-CD22 as disclosed in U.S. Pat. No. 7,355,012.

In any of these or other embodiments described herein, the V_{L} and V_{H} domains may be arranged in either orientation and may be separated by up to about a 30 amino acid linker as disclosed herein or any other amino acid sequence capable of providing a spacer function compatible with interaction of the two sub-binding domains. In certain embodiments, a linker joining the V_{L} and V_{H} domains comprises an amino acid sequence as set forth in SEQ ID NO: 18-141, such as Linker 46 (SEQ ID NO:63), 130 (SEQ ID NO:138), or 131 (SEQ ID NO:139). Multi-specific binding domains can have at least two specific sub-binding domains, by analogy to camelid antibody organization, or at least four specific sub-binding domains, by analogy to the more conventional mammalian antibody organization of paired V_{L} and V_{H} chains.

In further embodiments, binding domains specific for a CD22 of this disclosure may comprise one or more complementarity determining region (“CDR”), or multiple copies of one or more such CDRs, which have been obtained, derived, or designed from variable regions of anti-CD22 disclosed in U.S. Pat. No. 7,355,012, scFv or Fab fragment or from heavy or light chain variable regions thereof. Thus, a binding domain of this disclosure can comprise a single CDR from a variable region of anti-CD22 of U.S. Pat. No. 7,355,012, or it can comprise multiple CDRs that can be the same or different. In certain embodiments, binding domains of this disclosure comprise V_{L} and V_{H} domains specific for a CD22 comprising framework regions and CDR1, CDR2 and CDR3 regions, wherein (a) the V_{H} domain comprises the amino acid sequence of a heavy chain CDR3; or (b) the V_{L} domain comprises the amino acid sequence of a light chain CDR3; or (c) the binding domain comprises a V_{H} amino acid sequence of (a) and a V_{L} amino acid sequence of (b). In any of the embodiments described herein comprising specific CDRs against a CD22, a binding domain can comprise (i) a V_{H} domain having an amino acid sequence that is at least 80%, 85%, 90%, 91%, 92%, 93%, 94%, 95%, 96%, 97%, 98%, or 99° A identical to the amino acid sequence of a V_{H} domain disclosed in U.S. Pat. No. 7,355,012, wherein each CDR can have zero changes or no more than one, two, or three amino acid changes (i.e., many of the changes will be in the framework); or (ii) a V_{L} domain having an amino acid sequence that is at least 80%, 85%, 90%, 91%, 92%, 93%, 94%, 95%, 96%, 97%, 98%, or 99% identical to the amino acid sequence of a V_{L} domain disclosed in U.S. Pat. No. 7,355,012, wherein each CDR can have zero changes or no more than one, two, or three amino acid changes (i.e., many of the changes will be in the framework); or (iii) both a V_{H} domain of (i) and a V_{L} domain of (ii).

### CD32b Binding Domains

As noted above, in certain embodiments the present disclosure provides polypeptides containing a binding region or domain specific for CD32b. In certain embodiments, the binding domain is a CD32b antagonist (i.e., can inhibit CD32b signaling) or agonist. Exemplary CD32b antagonists or agonists include binding domains specific for a CD32b, such as an immunoglobulin variable binding domain or derivative thereof (e.g., an antibody, Fab, scFv, or the like).

CD32b, also known as FCGR2B, is a target for deregulation through chromosomal translocation in lymphoma and, specifically, dysregulation may play a role in tumor progression in follicular lymphoma (Callalan et al. (2000) Proc. Nat'l. Acad. Sci. USA 97:309). CD32b is expressed as four different isoforms (isoform 1, GenBank Accession No. NP_{—}003992.3, 310 amino acids; isoform 2, GenBank Accession No. NP_{—}001002273.1, 290 amino acids, isoform 3, GenBank Accession No. NP_{—}001002274.1, 291 amino acids; and isoform 4, GenBank Accession No. NP_{—}001002275.1, 309 amino acids).

In some embodiments, binding domains of this disclosure comprise V_{L} and V_{H} domains specific for a CD32b as described herein. In certain embodiments, there are provided polypeptide binding domains specific for a CD32b comprising a sequence that is at least 90%, at least 91%, at least 92%, at least 93%, at least 94%, at least 95%, at least 96%, at least 97%, at least 98%, at least 99%, at least 99.5%, or at least 100% identical to a light chain variable region (V_{L}) or to a heavy chain variable region (V_{H}), or both, wherein each CDR can have zero changes or no more than one, two, or three amino acid changes (i.e., many of the changes will be in the framework), from a human anti-CD32b.

In any of these or other embodiments described herein, the V_{L} and V_{H} domains may be arranged in either orientation and may be separated by up to about a 30 amino acid linker as disclosed herein or any other amino acid sequence capable of providing a spacer function compatible with interaction of the two sub-binding domains. In certain embodiments, a linker joining the V_{L} and V_{H} domains comprises an amino acid sequence as set forth in SEQ ID NO: 18-141, such as Linker 46 (SEQ ID NO:63), 130 (SEQ ID NO:138), or 131 (SEQ ID NO:139). Multi-specific binding domains can have at least two specific sub-binding domains, by analogy to camelid antibody organization, or at least four specific sub-binding domains, by analogy to the more conventional mammalian antibody organization of paired V_{L} and V_{H} chains.

In further embodiments, binding domains specific for a CD32b of this disclosure may comprise one or more complementarity determining region (“CDR”), or multiple copies of one or more such CDRs, which have been obtained, derived, or designed from variable regions of anti-CD32b, scFv or Fab fragment or from heavy or light chain variable regions thereof. Thus, a binding domain of this disclosure can comprise a single CDR from a variable region of anti-CD32b, or it can comprise multiple CDRs that can be the same or different. In certain embodiments, binding domains of this disclosure comprise V_{L} and V_{H} domains specific for a CD32b comprising framework regions and CDR1, CDR2 and CDR3 regions, wherein (a) the V_{H} domain comprises the amino acid sequence of a heavy chain CDR3; or (b) the V_{L} domain comprises the amino acid sequence of a light chain CDR3; or (c) the binding domain comprises a V_{H} amino acid sequence of (a) and a V_{L} amino acid sequence of (b). In any of the embodiments described herein comprising specific CDRs against a CD32b, a binding domain can comprise (i) a V_{H} domain having an amino acid sequence that is at least 80%, 85%, 90%, 91%, 92%, 93%, 94%, 95%, 96%, 97%, 98%, or 99% identical to the amino acid sequence of a V_{H} domain, wherein each CDR can have zero changes or no more than one, two, or three amino acid changes (i.e., many of the changes will be in the framework); or (ii) a V_{L} domain having an amino acid sequence that is at least 80%, 85%, 90%, 91%, 92%, 93%, 94%, 95%, 96%, 97%, 98%, or 99% identical to the amino acid sequence of a V_{L} domain, wherein each CDR can have zero changes or no more than one, two, or three amino acid changes (i.e., many of the changes will be in the framework); or (iii) both a V_{H} domain of (i) and a V_{L} domain of (ii).

### CD267 Binding Domains

As noted above, in certain embodiments the present disclosure provides polypeptides containing a binding region or domain that is specific for CD267. In certain embodiments, the binding domain is a CD267 antagonist (i.e., can inhibit CD267 signaling) or agonist. Exemplary CD267 antagonists or agonists include binding domains specific for a CD267, such as an immunoglobulin variable binding domain or derivative thereof (e.g., an antibody, Fab, scFv, or the like).

CD267 (GenBank Accession No. NP_{—}036584; also known as TACI; TNFRSF13B) is a lymphocyte-specific member of the tumor necrosis factor (TNF) receptor superfamily that interacts with calcium-modulator and cyclophilin ligand (CAML). It is 293 amino acids in length with two cysteine-rich TNFR repeats at amino acids 34-66 and 71-104, and a transmembrane domain at amino acids 167-186. CD267 induces activation of the transcription factors NFAT, AP1, and NF-kappa-B and plays a role in the development of B-cell autoimmunity by interacting with the TNF ligands APRIL and BAFF (Gross et al. (2000) Nature 404:995-9). A soluble form of the CD267 extracellular domain has been shown to prolong survival in an amino model of SLE (Gross et al. Ibid) and to reduce inflammation and the rate of occurrence of disease in a mouse model of collagen-induced arthritis (Gross et al. (2001) Immunity 15:289-302).

In some embodiments, binding domains of this disclosure comprise V_{L} and V_{H} domains specific for a CD267 as described herein. In certain embodiments, there are provided polypeptide binding domains specific for a CD267 comprising a sequence that is at least 90%, at least 91%, at least 92%, at least 93%, at least 94%, at least 95%, at least 96%, at least 97%, at least 98%, at least 99%, at least 99.5%, or at least 100% identical to a light chain variable region (V_{L}) or to a heavy chain variable region (V_{H}), or both, wherein each CDR can have zero changes or no more than one, two, or three amino acid changes (i.e., many of the changes will be in the framework), from a human anti-CD267.

In any of these or other embodiments described herein, the V_{L} and V_{H} domains may be arranged in either orientation and may be separated by up to about a 30 amino acid linker as disclosed herein or any other amino acid sequence capable of providing a spacer function compatible with interaction of the two sub-binding domains. In certain embodiments, a linker joining the V_{L} and V_{H} domains comprises an amino acid sequence as set forth in SEQ ID NO: 18-141, such as Linker 46 (SEQ ID NO:63), 130 (SEQ ID NO:138), or 131 (SEQ ID NO:139). Multi-specific binding domains can have at least two specific sub-binding domains, by analogy to camelid antibody organization, or at least four specific sub-binding domains, by analogy to the more conventional mammalian antibody organization of paired V_{L} and V_{H} chains.

In further embodiments, binding domains specific for a CD267 of this disclosure may comprise one or more complementarity determining region (“CDR”), or multiple copies of one or more such CDRs, which have been obtained, derived, or designed from variable regions of an anti-CD267, scFv or Fab fragment or from heavy or light chain variable regions thereof. Thus, a binding domain of this disclosure can comprise a single CDR from a variable region of an anti-CD267, or it can comprise multiple CDRs that can be the same or different. In certain embodiments, binding domains of this disclosure comprise V_{L} and V_{H} domains specific for a CD267 comprising framework regions and CDR1, CDR2 and CDR3 regions, wherein (a) the V_{H} domain comprises the amino acid sequence of a heavy chain CDR3; or (b) the V_{L} domain comprises the amino acid sequence of a light chain CDR3; or (c) the binding domain comprises a V_{H} amino acid sequence of (a) and a V_{L} amino acid sequence of (b). In any of the embodiments described herein comprising specific CDRs against a CD269, a binding domain can comprise (i) a V_{H} domain having an amino acid sequence that is at least 80%, 85%, 90%, 91%, 92%, 93%, 94%, 95%, 96%, 97%, 98%, or 99% identical to the amino acid sequence of a V_{H} domain of an anti-CD267, wherein each CDR can have zero changes or no more than one, two, or three amino acid changes (i.e., many of the changes will be in the framework); or (ii) a V_{L} domain having an amino acid sequence that is at least 80%, 85%, 90%, 91%, 92%, 93%, 94%, 95%, 96%, 97%, 98%, or 99% identical to the amino acid sequence of a V_{L} domain of an anti-CD267, wherein each CDR can have zero changes or no more than one, two, or three amino acid changes (i.e., many of the changes will be in the framework); or (iii) both a V_{H} domain of (i) and a V_{L} domain of (ii).

### CD269 Binding Domains

As noted above, in certain embodiments the present disclosure provides polypeptides containing a binding region or domain that is specific for CD269. In certain embodiments, the binding domain is a CD269 antagonist (i.e., can inhibit CD269 signaling) or agonist. Exemplary CD269 antagonists or agonists include binding domains specific for a CD269, such as an immunoglobulin variable binding domain or derivative thereof (e.g., an antibody, Fab, scFv, or the like).

CD269 (GenBank Accession No. NP_{—}001183; also known as TNFRSF17, or BCMA) is a member of the TNF-receptor superfamily that is preferentially expressed in mature B lymphocytes. It is believed to be important for B cell development and autoimmune response. CD269 has been shown to bind to the tumor necrosis factor superfamily, member 13b (TNFSF13B; also known as TALL-1 or BAFF) and to a proliferation inducing ligand (APRIL), both of which have been shown to promote tumor cell survival. In addition, studies by Nagatani et al. ((2007) Arthritis Rheum. 56:3554-63) have indicated that APRIL plays a major role in the pathogenesis of rheumatoid arthritis, and BAFF has been implicated in the development of B-cell autoimmune disease (Gross et al. (2000) Nature 404:995-9). A soluble form of CD269 has been shown to inhibit tumor cell growth in Nu/Nu mice implanted with HT29 and A549 tumor cells (Rennert et al. (2000) J. Exp. Med. 192:1677-1683).

In some embodiments, binding domains of this disclosure comprise V_{L} and V_{H} domains specific for a CD269 as described herein. In certain embodiments, there are provided polypeptide binding domains specific for a CD269 comprising a sequence that is at least 90%, at least 91%, at least 92%, at least 93%, at least 94%, at least 95%, at least 96%, at least 97%, at least 98%, at least 99%, at least 99.5%, or at least 100% identical to a light chain variable region (V_{L}) or to a heavy chain variable region (V_{H}), or both, wherein each CDR can have zero changes or no more than one, two, or three amino acid changes (i.e., many of the changes will be in the framework), from a human anti-CD269.

In any of these or other embodiments described herein, the V_{L} and V_{H} domains may be arranged in either orientation and may be separated by up to about a 30 amino acid linker as disclosed herein or any other amino acid sequence capable of providing a spacer function compatible with interaction of the two sub-binding domains. In certain embodiments, a linker joining the V_{L} and V_{H} domains comprises an amino acid sequence as set forth in SEQ ID NO: 18-141, such as Linker 46 (SEQ ID NO:63), 130 (SEQ ID NO:138), or 131 (SEQ ID NO:139). Multi-specific binding domains can have at least two specific sub-binding domains, by analogy to camelid antibody organization, or at least four specific sub-binding domains, by analogy to the more conventional mammalian antibody organization of paired V_{L} and V_{H} chains.

In further embodiments, binding domains specific for a CD269 of this disclosure may comprise one or more complementarity determining region (“CDR”), or multiple copies of one or more such CDRs, which have been obtained, derived, or designed from variable regions of an anti-CD269, scFv or Fab fragment or from heavy or light chain variable regions thereof. Thus, a binding domain of this disclosure can comprise a single CDR from a variable region of an anti-CD269, or it can comprise multiple CDRs that can be the same or different. In certain embodiments, binding domains of this disclosure comprise V_{L} and V_{H} domains specific for a CD269 comprising framework regions and CDR1, CDR2 and CDR3 regions, wherein (a) the V_{H} domain comprises the amino acid sequence of a heavy chain CDR3; or (b) the V_{L} domain comprises the amino acid sequence of a light chain CDR3; or (c) the binding domain comprises a V_{H} amino acid sequence of (a) and a V_{L} amino acid sequence of (b). In any of the embodiments described herein comprising specific CDRs against a CD269, a binding domain can comprise (i) a V_{H} domain having an amino acid sequence that is at least 80%, 85%, 90%, 91%, 92%, 93%, 94%, 95%, 96%, 97%, 98%, or 99% identical to the amino acid sequence of a V_{H} domain of an anti-CD269, wherein each CDR can have zero changes or no more than one, two, or three amino acid changes (i.e., many of the changes will be in the framework); or (ii) a V_{L} domain having an amino acid sequence that is at least 80%, 85%, 90%, 91%, 92%, 93%, 94%, 95%, 96%, 97%, 98%, or 99% identical to the amino acid sequence of a V_{L} domain of an anti-CD269, wherein each CDR can have zero changes or no more than one, two, or three amino acid changes (i.e., many of the changes will be in the framework); or (iii) both a V_{H} domain of (i) and a V_{I}, domain of (ii).

### Multi-Specific Fusion Proteins

The present disclosure provides multi-specific fusion proteins comprising a domain that binds to CD100 or other CD72 ligand (“CD72-ligand binding domain”) and a domain that binds a molecule other than a CD72 ligand (“heterologous binding domain”), such as FCRL1, FCRL2, FCRL3, FCRL4, FCRL5, FCRL6, CD19, CD20, CD32b, CD37, CD79a, CD79b, CD267 or CD269. It is contemplated that a CD72-ligand binding domain may be at the amino-terminus and the heterologous binding domain at the carboxy-terminus of a fusion protein, or the heterologous binding domain may be at the amino-terminus and the CD72-ligand binding domain may be at the carboxy-terminus. As set forth herein, the binding domains of this disclosure may be fused to each end of an intervening domain (e.g., an immunoglobulin constant region or sub-region thereof). Furthermore, the two or more binding domains may be each joined to an intervening domain via a linker known in the art or as described herein.

As used herein, an “intervening domain” refers to an amino acid sequence that simply functions as a scaffold for one or more binding domains so that the fusion protein will exist primarily (e.g., 50% or more of a population of fusion proteins) or substantially (e.g., 90% or more of a population of fusion proteins) as a single chain polypeptide in a composition. For example, certain intervening domains can have a structural function (e.g., spacing, flexibility, rigidity) or biological function (e.g., an increased half-life in plasma, such as in human blood). Exemplary intervening domains that can increase half-life of the fusion proteins of this disclosure in plasma include albumin, transferrin, a scaffold domain that binds a serum protein, or the like, or fragments thereof.

In certain embodiments, the intervening domain contained in a multi-specific fusion protein of this disclosure is a “dimerization domain,” which refers to an amino acid sequence that is capable of promoting the association of at least two single chain polypeptides or proteins via non-covalent or covalent interactions, such as by hydrogen bonding, electrostatic interactions, Van der Waal's forces, disulfide bonds, hydrophobic interactions, or the like, or any combination thereof. Exemplary dimerization domains include immunoglobulin heavy chain constant regions or sub-regions. It should be understood that a dimerization domain can promote the formation of dimers or higher order multimer complexes (such as trimers, tetramers, pentamers, hexamers, septamers, octamers, etc.).

A “constant sub-region” is a term defined herein to refer to a preferred peptide, polypeptide, or protein sequence that corresponds to or is derived from part or all of one or more immunoglobulin constant region domains, but not all constant region domains found in a source antibody. In some embodiments, the constant region domains of a fusion protein of this disclosure may lack or have minimal effector functions of antibody-dependent cell-mediated cytotoxicity (ADCC), antibody-dependent cell-mediated phagocytosis (ADCP) and complement activation and complement-dependent cytotoxicity (CDC), while retaining the ability to bind some F_{C} receptors (such as F_{C}Rn binding) and retaining a relatively long half life in vivo. In certain embodiments, a binding domain of this disclosure is fused to a human IgG1 constant region or sub-region, wherein the IgG1 constant region or sub-region has one or more of the following amino acids mutated: leucine at position 234 (L234), leucine at position 235 (L235), glycine at position 237 (G237), glutamate at position 318 (E318), lysine at position 320 (K320), lysine at position 322 (K322), or any combination thereof (EU numbering). For example, any one or more of these amino acids can be changed to alanine. In a further embodiment, an IgG1 Fc domain has each of L234, L235, G237, E318, K320, and K322 (according to EU numbering) mutated to an alanine (i.e., L234A, L235A, G237A, E318A, K320A, and K322A, respectively).

Methods are known in the art for making mutations inside or outside an Fc domain that can alter Fc interactions with Fc receptors (CD16, CD32, CD64, CD89, FcεR1, FcRn) or with the complement component C1q (see, e.g., U.S. Pat. No. 5,624,821; Presta (2002) Curr. Pharma. Biotechnol. 3:237). Particular embodiments of this disclosure include compositions comprising immunoglobulin or fusion proteins that have a constant region or sub-region from human IgG wherein binding to FcRn and protein A are preserved and wherein the Fc domain no longer interacts or minimally interacts with other Fc receptors or C1q. For example, a binding domain of this disclosure can be fused to a human IgG1 constant region or sub-region wherein the asparagine at position 297 (N297 under the Kabat numbering) has been mutated to another amino acid to reduce or eliminate glycosylation at this site and, therefore, abrogate efficient Fc binding to FcγR and C1q. Another exemplary mutation is a P331S, which knocks out C1q binding but does not affect Fc binding.

In further embodiments, an immunoglobulin Fc region may have an altered glycosylation pattern relative to an immunoglobulin referent sequence. For example, any of a variety of genetic techniques may be employed to alter one or more particular amino acid residues that form a glycosylation site (see Co et al. (1993) Mol. Immunol. 30:1361; Jacquemon et al. (2006) J. Thromb. Haemost. 4:1047; Schuster et al. (2005) Cancer Res. 65:7934; Warnock et al. (2005) Biotechnol. Bioeng. 92:831). Alternatively, the host cells in which fusion proteins of this disclosure are produced may be engineered to produce an altered glycosylation pattern. One method known in the art, for example, provides altered glycosylation in the form of bisected, non-fucosylated variants that increase ADCC. The variants result from expression in a host cell containing an oligosaccharide-modifying enzyme. Alternatively, the Potelligent technology of BioWa/Kyowa Hakko is contemplated to reduce the fucose content of glycosylated molecules according to this disclosure. In one known method, a CHO host cell for recombinant immunoglobulin production is provided that modifies the glycosylation pattern of the immunoglobulin Fc region, through production of GDP-fucose.

Alternatively, chemical techniques are used to alter the glycosylation pattern of fusion proteins of this disclosure. For example, a variety of glycosidase and/or mannosidase inhibitors provide one or more of desired effects of increasing ADCC activity, increasing Fc receptor binding, and altering glycosylation pattern. In certain embodiment, cells expressing a multispecific fusion protein of the instant disclosure (containing a CD72 binding domain linked to a heterologous B cell specific target binding domain, such as a FCRL1-6, CD19, CD20, CD22, CD32b, CD37, CD79a, CD79b, CD267 or CD269 binding domain) are grown in a culture medium comprising a carbohydrate modifier at a concentration that increases the ADCC of immunoglycoprotein molecules produced by said host cell, wherein said carbohydrate modifier is at a concentration of less than 800 μM. In a preferred embodiment, the cells expressing these multispecific fusion proteins are grown in a culture medium comprising castanospermine or kifunensine, more preferably castanospermine at a concentration of 100-800 μM, such as 100 μM, 200 μM, 300 μM, 400 μM, 500 μM, 600 μM, 700 μM, or 800 μM. Methods for altering glycosylation with a carbohydrate modifier such as castanospermine are provided in US Patent Application Publication No. 2009/0041756 or PCT Publication No. WO 2008/052030.

In another embodiment, the immunoglobulin Fc region may have amino acid modifications that affect binding to effector cell Fc receptors. These modifications can be made using any technique known in the art, such as the approach disclosed in Presta et al. (2001) Biochem. Soc. Trans. 30:487. In another approach, the Xencor XmAb technology is available to engineer constant sub-regions corresponding to Fc domains to enhance cell killing effector function (see Lazar et al. (2006) Proc. Nat'l. Acad. Sci. (USA) 103:4005). Using this approach, for example, one can generate constant sub-regions with improved specificity and binding for FCγR, thereby enhancing cell killing effector function.

In still further embodiments, a constant region or sub-region can optionally increase plasma half-life or placental transfer in comparison to a corresponding fusion protein lacking such an intervening domain. In certain embodiments, the extended plasma half-life of a fusion protein of this disclosure is at least two, at least three, at least four, at least five, at least ten, at least 12, at least 18, at least 20, at least 24, at least 30, at least 36, at least 40, at least 48 hours, at least several days, at least a week, at least two weeks, at least several weeks, at least a month, at least two months, at least several months, or more in a human.

A constant sub-region may include part or all of any of the following domains: a C_{H2} domain and a C_{H3} domain (IgA, IgD, IgG), or a C_{H3} domain and a C_{H4} domain (IgE or IgM). A constant sub-region as defined herein, therefore, can refer to a polypeptide that corresponds to a portion of an immunoglobulin constant region. The constant sub-region may comprise a C_{H2} domain and a C_{H3} domain derived from the same, or different, immunoglobulins, antibody isotypes, or allelic variants. In some embodiments, the C_{H3} domain is truncated and comprises a carboxy-terminal sequence listed in US Patent Publication No. US 2009/0175867 (which is a CIP of PCT/US2007/071052) as SEQ ID NOS:366-371, which sequences are hereby incorporated by reference. In certain embodiments, a constant sub-region of a polypeptide of this disclosure has a C_{H2} domain and C_{H3} domain, which may optionally have an amino-terminal linker, a carboxy-terminal linker, or a linker at both ends.

A “linker” is a peptide that joins or links other peptides or polypeptides, such as a linker of about 2 to about 150 amino acids. In fusion proteins of this disclosure, a linker can join an intervening domain (e.g., an immunoglobulin-derived constant sub-region) to a binding domain or a linker can join two variable regions of a binding domain. For example, a linker can be an amino acid sequence obtained, derived, or designed from an antibody hinge region sequence, a sequence linking a binding domain to a receptor, or a sequence linking a binding domain to a cell surface transmembrane region or membrane anchor. In some embodiments, a linker can have at least one cysteine capable of participating in at least one disulfide bond under physiological conditions or other standard peptide conditions (e.g., peptide purification conditions, conditions for peptide storage). In certain embodiments, a linker corresponding or similar to an immunoglobulin hinge peptide retains a cysteine that corresponds to the hinge cysteine disposed toward the amino-terminus of that hinge. In further embodiments, a linker is from an IgG1 or IgG2A hinge and has one cysteine or two cysteines corresponding to hinge cysteines. In certain embodiments, one or more disulfide bonds are formed as inter-chain disulfide bonds between intervening domains. In other embodiments, fusion proteins of this disclosure can have an intervening domain fused directly to a binding domain (i.e., absent a linker or hinge). In some embodiments, the intervening domain is a dimerization domain.

The intervening or dimerization domain of multi-specific fusion proteins of this disclosure may be connected to one or more terminal binding domains by a peptide linker. In addition to providing a spacing function, a linker can provide flexibility or rigidity suitable for properly orienting the one or more binding domains of a fusion protein, both within the fusion protein and between or among the fusion proteins and their target(s). Further, a linker can support expression of a full-length fusion protein and stability of the purified protein both in vitro and in vivo following administration to a subject in need thereof, such as a human, and is preferably non-immunogenic or poorly immunogenic in those same subjects. In certain embodiments, a linker of an intervening or a dimerization domain of multi-specific fusion proteins of this disclosure may comprise part or all of a human immunoglobulin hinge.

Additionally, a binding domain may comprise a V_{H} and a V_{L} domain, and these variable region domains may be combined by a linker. Exemplary variable region binding domain linkers include those belonging to the (GlyₙSer) family, such as (Gly₃Ser)ₙ(Gly₄Ser)₁, (Gly₃Ser)₁(Gly₄Ser)ₙ, (Gly₃Ser)ₙ(Gly₄Ser)ₙ, or (Gly₄Ser)ₙ, wherein n is an integer of 1 to 5 (see, e.g., Linkers 22, 29, 46, 89, 90, 116, 130, and 131 corresponding to SEQ ID NOS:39, 46, 63, 106, 107, 124, 138 and 139, respectively). In preferred embodiments, these (GlyₙSer)-based linkers are used to link variable domains and are not used to link a binding domain to an intervening domain.

Exemplary linkers that can be used join an intervening domain (e.g., an immunoglobulin-derived constant sub-region) to a binding domain or to join two variable regions of a binding domain are set forth in SEQ ID NO: 18-141.

Linkers contemplated in this disclosure include, for example, peptides derived from any inter-domain region of an immunoglobulin superfamily member (e.g., an antibody hinge region) or a stalk region of C-type lectins, a family of type II membrane proteins. These linkers range in length from about two to about 150 amino acids, or about two to about 40 amino acids, or about eight to about 20 amino acids, preferably about ten to about 60 amino acids, more preferably about 10 to about 30 amino acids, and most preferably about 15 to about 25 amino acids. For example, Linker 1 (SEQ ID NO: 18) is two amino acids in length and Linker 119 (SEQ ID NO: 127) is 36 amino acids in length.

Beyond general length considerations, a linker suitable for use in the fusion proteins of this disclosure includes an antibody hinge region selected from an IgG hinge, IgA hinge, IgD hinge, IgE hinge, or variants thereof. In certain embodiments, a linker may be an antibody hinge region (upper and core region) selected from human IgG1, human IgG2, human IgG3, human IgG4, or fragments or variants thereof. As used herein, a linker that is an “immunoglobulin hinge region” refers to the amino acids found between the carboxyl end of CH1 and the amino terminal end of CH2 (for IgG, IgA, and IgD) or the amino terminal end of CH3 (for IgE and IgM). A “wild type immunoglobulin hinge region,” as used herein, refers to a naturally occurring amino acid sequence interposed between and connecting the CH1 and CH2 regions (for IgG, IgA, and IgD) or interposed between and connecting the CH2 and CH3 regions (for IgE and IgM) found in the heavy chain of an antibody. In preferred embodiments, the wild type immunoglobulin hinge region sequences are human.

According to crystallographic studies, an IgG hinge domain can be functionally and structurally subdivided into three regions: the upper hinge region, the core or middle hinge region, and the lower hinge region (Shin et al., Immunological Reviews 130:87 (1992)). Exemplary upper hinge regions include EPKSCDKTHT (SEQ ID NO:151) as found in IgG1, ERKCCVE (SEQ ID NO:152) as found in IgG2, ELKTPLGDTT HT (SEQ ID NO:153) or EPKSCDTPPP (SEQ ID NO:154) as found in IgG3, and ESKYGPP (SEQ ID NO:155) as found in IgG4. Exemplary middle hinge regions include CPPCP (SEQ ID NO:156) as found in IgG1 and IgG2, CPRCP (SEQ ID NO:157) as found in IgG3, and CPSCP (SEQ ID NO:158) as found in IgG4. While IgG1, IgG2, and IgG4 antibodies each appear to have a single upper and middle hinge, IgG3 has four in tandem—one of ELKTPLGDTT HTCPRCP (SEQ ID NO:159) and three of EPKSCDTPPP CPRCP (SEQ ID NO:160).

IgA and IgD antibodies appear to lack an IgG-like core region, and IgD appears to have two upper hinge regions in tandem (see SEQ ID NOS:161 and 162). Exemplary wild type upper hinge regions found in IgA1 and IgA2 antibodies are set forth in SEQ ID NOS:163 and 164.

IgE and IgM antibodies, in contrast, instead of a typical hinge region have a CH2 region with hinge-like properties. Exemplary wild-type CH2 upper hinge-like sequences of IgE and IgM are set forth in SEQ ID NO:165 and SEQ ID NO:166, respectively.

An “altered wild type immunoglobulin hinge region” or “altered immunoglobulin hinge region” refers to (a) a wild type immunoglobulin hinge region with up to 30% amino acid changes (e.g., up to 25%, 20%, 15%, 10%, or 5% amino acid substitutions or deletions), (b) a portion of a wild type immunoglobulin hinge region that is at least 10 amino acids (e.g., at least 12, 13, 14 or 15 amino acids) in length with up to 30% amino acid changes (e.g., up to 25%, 20%, 15%, 10%, or 5% amino acid substitutions or deletions), or (c) a portion of a wild type immunoglobulin hinge region that comprises the core hinge region (which portion may be 4, 5, 6, 7, 8, 9, 10, 11, 12, 13, 14, or 15, or at least 4, 5, 6, 7, 8, 9, 10, 11, 12, 13, 14, or 15 amino acids in length). In certain embodiments, one or more cysteine residues in a wild type immunoglobulin hinge region may be substituted by one or more other amino acid residues (e.g., one or more serine residues). An altered immunoglobulin hinge region may alternatively or additionally have a proline residue of a wild type immunoglobulin hinge region substituted by another amino acid residue (e.g., a serine residue).

Alternative hinge and linker sequences that can be used as connecting regions may be crafted from portions of cell surface receptors that connect IgV-like or IgC-like domains. Regions between IgV-like domains where the cell surface receptor contains multiple IgV-like domains in tandem and between IgC-like domains where the cell surface receptor contains multiple tandem IgC-like regions could also be used as connecting regions or linker peptides. In certain embodiments, hinge and linker sequences are from five to 60 amino acids long, and may be primarily flexible, but may also provide more rigid characteristics, and may contain primarily an α-helical structure with minimal β-sheet structure. Preferably, sequences are stable in plasma and serum and are resistant to proteolytic cleavage. In some embodiments, sequences may contain a naturally occurring or added motif such as CPPC that confers the capacity to form a disulfide bond or multiple disulfide bonds to stabilize the C-terminus of the molecule. In other embodiments, sequences may contain one or more glycosylation sites. Examples of hinge and linker sequences include interdomain regions between the IgV-like and IgC-like or between the IgC-like or IgV-like domains of CD2, CD4, CD22, CD33, CD48, CD58, CD66, CD80, CD86, CD96, CD150, CD166, and CD244. Alternative hinges may also be crafted from disulfide-containing regions of Type II receptors from non-immunoglobulin superfamily members such as CD69, CD72, and CD161.

In some embodiments, a hinge linker has a single cysteine residue for formation of an interchain disulfide bond. In other embodiments, a linker has two cysteine residues for formation of interchain disulfide bonds. In further embodiments, a linker is derived from an immunoglobulin interdomain region (e.g., an antibody hinge region) or a Type II C-type lectin stalk region (derived from a Type II membrane protein; see, e.g., exemplary lectin stalk region sequences set forth in of PCT Application Publication No. WO 2007/146968, such as SEQ ID NOS:111, 113, 115, 117, 119, 121, 123, 125, 127, 129, 131, 133, 135, 149, 151, 153, 155, 157, 159, 161, 163, 165, 167, 169, 231, 233, 235, 237, 239, 241, 243, 245, 247, 249, 251, 253, 255, 257, 259, 261, 263, 265, 267, 269, 271, 273, 275, 277, 279, 281, 287, 289, 297, 305, 307, 309-311, 313-331, 346, 373-377, 380, or 381 from that publication, which sequences are hereby incorporated by reference).

In one aspect, exemplary multi-specific fusion proteins containing a CD72-ligand binding domain as described herein will also contain at least one additional binding region or domain that is specific for a target other than a CD72 ligand, such as a B-cell specific surface protein. For example, a multi-specific fusion protein of this disclosure has a CD72-ligand binding domain linked to a FCRL1, FCRL2, FCRL3, FCRL4, FCRL5, FCRL6, CD19, CD20, CD22, CD32b, CD37, CD79a, CD79b, CD267 or CD269 binding domain by an intervening domain. In certain embodiments, a multi-specific fusion protein comprises a first and second binding domain, a first and second linker, and an intervening domain, wherein one end of the intervening domain is fused via the first linker to a first binding domain that is a CD72-ligand binding domain (e.g., a CD72 ectodomain, an anti-CD100) and at the other end is fused via the second linker to a different binding domain that is specific for a B-cell surface protein (e.g., an immunoglobulin variable region specific for a FCRL1, FCRL2, FCRL3, FCRL4, FCRL5, FCRL6, CD19, CD20, CD22, CD32b, CD37, CD79a, CD79b, CD267 or CD269).

In certain embodiments, the first linker and second linker of a multi-specific fusion protein of this disclosure are each independently selected from, for example, SEQ ID NO: 18-141. For example, the first or second linker can be any one of Linkers 47, 58, 126-131 (SEQ ID NOS:64, 75, 134-139, respectively) or any combination thereof. In further examples, one linker is Linker 47 (SEQ ID NO:64) or Linker 132 (SEQ ID NO:140) and the other linker is Linker 127 (SEQ ID NO:135), or one linker is Linker 58 (SEQ ID NO:75) or Linker 133 (SEQ ID NO:141) and the other linker is Linker 126 (SEQ ID NO:134), or one linker is Linker 58 (SEQ ID NO:75) or Linker 133 (SEQ ID NO:141) and the other linker is Linker 127 (SEQ ID NO:135), or one linker is Linker 58 (SEQ ID NO:75) or Linker 133 (SEQ ID NO:141) and the other linker is Linker 128 (SEQ ID NO:136), or one linker is Linker 58 (SEQ ID NO:75) or Linker 133 (SEQ ID NO:141) and the other linker is Linker 129 (SEQ ID NO:137). In further examples, binding domains of this disclosure that comprise V_{H} and V_{L} domains, such as those specific for any one of FCRL1, FCRL2, FCRL3, FCRL4, FCRL5, FCRL6, CD19, CD20, CD22, CD32b, CD37, CD79a, CD79b, CD267 or CD269, can have a further (third) linker between the V_{H} and V_{L} domains, such as Linker 46 (SEQ ID NO:63), Linker 130 (SEQ ID NO:138), or Linker 131 (SEQ ID NO:139). In any of these embodiments, the linkers may be flanked by one to five additional amino acids internally (e.g., Linker 131 has an alanine internal to the (G₄S) core sequence), on either end (e.g., Linker 130 has a serine on the amino-end of the (G₄S) core sequence) or on both ends (e.g., Linker 120 has two amino acids (asparagine-tyrosine) on one end and three amino acids (glycine-asparagine-serine) one the other end of the (G₄S) core sequence), which may simply be a result of creating such a recombinant molecule (e.g., use of a particular restriction enzyme site to join nucleic acid molecules may result in the insertion of one to several amino acids), and for purposes of this disclosure may be considered a part of any particular linker core sequence.

In further embodiments, the intervening domain of a multi-specific fusion protein of this disclosure is comprised of an immunoglobulin constant region or sub-region, wherein the intervening domain is disposed between a CD72-ligand binding domain and a binding domain specific for a B-cell specific protein. In certain embodiments, the intervening domain of a multi-specific fusion protein of this disclosure has a CD72-ligand binding domain at the amino-terminus and a binding domain specific for a B-cell specific protein at the carboxy-terminus. In other embodiments, the intervening domain of a multi-specific fusion protein of this disclosure has a binding domain specific for a B-cell specific protein at the amino-terminus and a CD72-ligand binding domain at the carboxy-terminus. In further embodiments, the immunoglobulin constant region sub-region includes CH2 and CH3 domains of immunoglobulin G1 (IgG1). In related embodiments, the IgG1 CH2 and CH3 domains have one or more of the following amino acids mutated (i.e., have a different amino acid at that position): leucine at position 234 (L234), leucine at position 235 (L235), glycine at position 237 (G237), glutamate at position 318 (E318), lysine at position 320 (K320), lysine at position 322 (K322), or any combination thereof (EU numbering). For example, any one of these amino acids can be changed to alanine. In a further embodiment, according to EU numbering, the CH2 domain has each of L234, L235, and G237 mutated to an alanine (i.e., L234A, L235A, and G237A, respectively), and the IgG1 CH3 domain has each of E318, K320, and K322 mutated to an alanine (i.e., E318A, K320A, and K322A, respectively).

In some embodiments, a multi-specific fusion protein of this disclosure has a CD72-ligand binding domain that comprises a CD72 extracellular domain or sub-domain, a CD72 C-type lectin domain, or a CD100-specific antibody-derived binding domains. In some embodiments, a CD72-ligand binding domain is an ectodomain of CD72. In certain embodiments, a CD72-ligand binding domain comprises a carboxy-terminal portion of CD72, such as the last 243 amino acids of CD72 as set forth in GenBank Accession No. NP_{—}001773.1 (SEQ ID NO:1). In other embodiments, a CD72-ligand binding domain comprises amino acids 200-359, 210-359, 221-359, or 233-359 of SEQ ID NO:1. In further embodiments, a CD72-ligand binding domain comprising amino acids 221-359 or 233-359 of SEQ ID NO:1 is fused to an intervening domain via linker that is a CD72 stalk region or a portion thereof, such as amino acids 117-232, 200-232, or 210-232 of SEQ ID NO:1.

In further embodiments, a multi-specific fusion protein of this disclosure has a CD72-ligand binding domain binding domain and a binding domain specific for a B-cell specific protein such as CD19, comprising (i) a V_{H} domain having an amino acid sequence that is at least 80%, 85%, 90%, 91%, 92%, 93%, 94%, 95%, 96%, 97%, 98%, 99% or 100% identical to the amino acid sequence of a V_{H} domain found in SEQ ID NO:9; or (ii) a V_{L} domain having an amino acid sequence that is at least 80%, 85%, 90%, 91%, 92%, 93%, 94%, 95%, 96%, 97%, 98%, 99% or 100% identical to the amino acid sequence of a V_{L} domain found in SEQ ID NO:9; or (iii) both a V_{H} domain of (i) and a V_{L} domain of (ii). In still further embodiments, a multi-specific fusion protein of this disclosure has a CD72-ligand binding domain binding domain and a binding domain specific for a B-cell specific protein such as CD37, comprising (i) a V_{H} domain having an amino acid sequence that is at least 80%, 85%, 90%, 91%, 92%, 93%, 94%, 95%, 96%, 97%, 98%, 99° A or 100% identical to the amino acid sequence of a V_{H} domain found in SEQ ID NO:11; or (ii) a V_{L} domain having an amino acid sequence that is at least 80%, 85%, 90%, 91%, 92%, 93%, 94%, 95%, 96%, 97%, 98%, 99% or 100% identical to the amino acid sequence of a V_{L} domain found in SEQ ID NO:11; or (iii) both a V_{H} domain of (i) and a V_{L} domain of (ii).

In yet further embodiments, a binding domain specific for a B-cell specific protein, comprises V_{H} and V_{L} domains comprising framework regions and CDR1, CDR2 and CDR3 regions, wherein (a) the V_{H} domain comprises the amino acid sequence of a heavy chain CDR1, CDR2, and CDR3 found in SEQ ID NO:9 or 11; or (b) the V_{L} domain comprises the amino acid sequence of a light chain CDR1, CDR2, and CDR3 found in SEQ ID NO:9 or 11. The V_{L} and V_{H} domains of these multi-specific fusion proteins may be arranged in either orientation and may be separated by up to about a 30 amino acid linker as disclosed herein. In certain embodiments, a linker joining the V_{H} and V_{L} domains comprises an amino acid sequence of Linker 47 (SEQ ID NO:64), Linker 130 (SEQ ID NO:138), or Linker 131 (SEQ ID NO:139).

Exemplary structures of such multi-specific fusion proteins, referred to herein as Xceptor molecules, include N-BD-X-ED-C, N-ED-X-BD-C, N-BD1-X-BD2-C, wherein N and C represent the amino-terminus and carboxy-terminus, respectively; BD is an immunoglobulin-like or immunoglobulin variable region binding domain, X is an intervening domain, and ED is a receptor extracellular or ectodomain, C-type lectin domain, or the like. In some constructs, X can comprise an immunoglobulin constant region or sub-region disposed between the first and second binding domains. In some embodiments, a multi-specific fusion protein of this disclosure has an intervening domain (X) comprising, from amino-terminus to carboxy-terminus, a structure as follows: -L1-X-L2-, wherein L1 and L2 are each independently a linker comprising from two to about 150 amino acids; and X is an immunoglobulin constant region or sub-region. In further embodiments, the multi-specific fusion protein will have an intervening domain is albumin, transferrin, or another serum protein binding protein, wherein the fusion protein remains primarily or substantially as a single chain polypeptide in a composition.

In still further embodiments, a multi-specific fusion protein of this disclosure has the following structure: N-BD1-X-L2-ED2-C, wherein ED2 is a CD72-ligand binding domain that is at least about 90% identical to an CD72 ectodomain; —X— is -L1-CH2CH3-, wherein L1 is a first IgG1 hinge, optionally mutated by substituting the first or second cysteine, and wherein —CH2CH3- is the CH2CH3 region of an IgG1 Fc domain; L2 is a linker selected from SEQ ID NO: 18-141; and BD2 is a binding domain specific for a B-cell specific protein, such as FCRL1, FCRL2, FCRL3, FCRL4, FCRL5, FCRL6, CD19, CD20, CD22 CD32b, CD37, CD79a, CD79b, CD267 or CD269, as described herein.

In particular embodiments, a multi-specific Xceptor fusion protein has (a) a CD72-ligand binding domain comprising an amino acid sequence at least 80% to 100% identical to amino acids 233-359 set forth in SEQ ID NO:1, and (b) a CD19 or CD37 binding domain, comprising a heavy chain variable region with CDR1, CD2, and CDR3 amino acid sequences at least 80% to 100% identical to a sequence set forth in SEQ ID NO:9 or 11, respectively, and a light chain variable region with CDR1, CDR2, and CDR3 amino acid sequences at least 80% to 100% identical to a sequence set forth in SEQ ID NO:9 or 11, respectively, wherein, from amino-terminus to carboxy-terminus or from carboxy-terminus to amino-terminus, (i) a CD72-ligand binding domain of (a) or a CD19 or CD37 binding domain of (b) is fused to a first linker, (ii) the first linker is fused to an immunoglobulin heavy chain constant region of CH2 and CH3 comprising amino acids 39 to 255 of SEQ ID NO:7, (iii) the CH2CH3 constant region polypeptide is fused to a second linker, and (iv) the second linker is fused to a CD72-ligand binding domain of (a) or a CD19 or CD37 binding domain of (b). In certain embodiments, the first linker is Linker 47 (SEQ ID NO:64), Linker 132 (SEQ ID NO:140) or Linker 133 (SEQ ID NO:131), the second linker is any one of Linkers 126-129 (SEQ ID NOS:134-137), and a further (third) linker between the CD19 or CD37 binding domain V_{H} and V_{L} domains is Linker 130 (SEQ ID NO:138) or Linker 131 (SEQ ID NO:139).

In still further embodiments, a multi-specific fusion protein of this disclosure has an amino acid sequence at least 80%, 85%, 90%, 91%, 92%, 93%, 94%, 95%, 96%, 97%, 98%, 99%, or 100% identical to a sequence set forth in any one of SEQ ID NOS:9, 11, 13, 15, and 17, with or without a leader peptide (i.e., the first 20 amino acids found in these sequences). In further embodiments, a multi-specific fusion protein of this disclosure has a CD72-ligand binding domain comprising amino acids 233-359 of SEQ ID NO:1 and a CD19 binding domain, comprising a V_{L} of SEQ ID NO:9 joined to a V_{H} of SEQ ID NO:9 via Linker 131 (SEQ ID NO:139), wherein the CD19 binding domain is joined to the amino-terminus of an intervening domain comprising an immunoglobulin heavy chain constant region of CH2 and CH3 comprising amino acids 39 to 255 of SEQ ID NO:7 via Linker 132 (SEQ ID NO:140) and the CD72-ligand binding domain is joined to the carboxy-terminus of an intervening domain via Linker 127 (SEQ ID NO:135). In one embodiment, the multi-specific fusion protein has an amino acid sequence as set forth in SEQ ID NO:9. In still further embodiments, a multi-specific fusion protein of this disclosure has a CD72-ligand binding domain comprising amino acids 233-359 of SEQ ID NO:1 and a CD37 binding domain, comprising a V_{L} of any one of SEQ ID NO:11, 13, 15 and 17 joined to a V_{H} of any one of SEQ ID NO: 11, 13, 15 and 17 via Linker 130 (SEQ ID NO:138), wherein the CD37 binding domain is joined to the amino-terminus of an intervening domain comprising an immunoglobulin heavy chain constant region of CH2 and CH3 comprising amino acids 39 to 255 of SEQ ID NO:7 via Linker 133 (SEQ ID NO:141) and the CD72-ligand binding domain is joined to the carboxy-terminus of an intervening domain via Linker 126, 127, 128, or 129 (SEQ ID NO:134, 135, 136 or 137). In certain embodiments, the multi-specific fusion protein has an amino acid sequence as set forth in SEQ ID NO:11, SEQ ID NO:13, SEQ ID NO:15, or SEQ ID NO:17.

### Making Multi-Specific Fusion Proteins

To efficiently produce any of the binding domain polypeptides or fusion proteins described herein, a leader peptide is used to facilitate secretion of expressed polypeptides and fusion proteins. Using any of the conventional leader peptides (signal sequences) is expected to direct nascently expressed polypeptides or fusion proteins into a secretory pathway and to result in cleavage of the leader peptide from the mature polypeptide or fusion protein at or near the junction between the leader peptide and the polypeptide or fusion protein. A particular leader peptide will be chosen based on considerations known in the art, such as using sequences encoded by polynucleotides that allow the easy inclusion of restriction endonuclease cleavage sites at the beginning or end of the coding sequence for the leader peptide to facilitate molecular engineering, provided that such introduced sequences specify amino acids that either do not interfere unacceptably with any desired processing of the leader peptide from the nascently expressed protein or do not interfere unacceptably with any desired function of a polypeptide or fusion protein molecule if the leader peptide is not cleaved during maturation of the polypeptides or fusion proteins. Exemplary leader peptides of this disclosure include natural leader sequences (i.e., those expressed with the native protein) or use of heterologous leader sequences, such as H₃N-MDFQVQIFSFLLISASVIMSRG(X)ₙ—CO₂H, wherein X is any amino acid and n is zero to three (SEQ ID NO:149) or H₃N-MEAPAQLLFLLLLWLPDTTG-CO₂H (SEQ ID NO:150).

As noted herein, variants and derivatives of binding domains, such as ectodomains, light and heavy variable regions, and CDRs described herein, are contemplated. In one example, insertion variants are provided wherein one or more amino acid residues supplement a specific binding agent amino acid sequence. Insertions may be located at either or both termini of the protein, or may be positioned within internal regions of the specific binding agent amino acid sequence. Variant products of this disclosure also include mature specific binding agent products, i.e., specific binding agent products wherein a leader or signal sequence is removed, and the resulting protein having additional amino terminal residues. The additional amino terminal residues may be derived from another protein, or may include one or more residues that are not identifiable as being derived from a specific protein. Polypeptides with an additional methionine residue at position −1 are contemplated, as are polypeptides of this disclosure with additional methionine and lysine residues at positions −2 and −1. Variants having additional Met, Met-Lys, or Lys residues (or one or more basic residues in general) are particularly useful for enhanced recombinant protein production in bacterial host cells.

As used herein, “amino acids” refer to a natural (those occurring in nature) amino acid, a substituted natural amino acid, a non-natural amino acid, a substituted non-natural amino acid, or any combination thereof. The designations for natural amino acids are herein set forth as either the standard one- or three-letter code. Natural polar amino acids include asparagine (Asp or N) and glutamine (Gln or Q); as well as basic amino acids such as arginine (Arg or R), lysine (Lys or K), histidine (His or H), and derivatives thereof; and acidic amino acids such as aspartic acid (Asp or D) and glutamic acid (Glu or E), and derivatives thereof. Natural hydrophobic amino acids include tryptophan (Trp or W), phenylalanine (Phe or F), isoleucine (Ile or I), leucine (Leu or L), methionine (Met or M), valine (Val or V), and derivatives thereof; as well as other non-polar amino acids such as glycine (GIy or G), alanine (Ala or A), proline (Pro or P), and derivatives thereof. Natural amino acids of intermediate polarity include serine (Ser or S), threonine (Thr or T), tyrosine (Tyr or Y), cysteine (Cys or C), and derivatives thereof. Unless specified otherwise, any amino acid described herein may be in either the D- or L-configuration.

Substitution variants include those fusion proteins wherein one or more amino acid residues in an amino acid sequence are removed and replaced with alternative residues. In some embodiments, the substitutions are conservative in nature; however, this disclosure embraces substitutions that are also non-conservative. Amino acids can be classified according to physical properties and contribution to secondary and tertiary protein structure. A conservative substitution is recognized in the art as a substitution of one amino acid for another amino acid that has similar properties. Exemplary conservative substitutions are set out in Table 1 (see WO 97/09433, page 10, published Mar. 13, 1997), immediately below.

TABLE 1
Conservative Substitutions I

| Side Chain | Characteristic | Amino Acid |
|---|---|---|
| Aliphatic | Non-polar | G, A, P, I, L, V |
| | Polar - uncharged | S, T, M, N, Q |
| | Polar - charged | D, E, K, R |
| Aromatic | | H, F, W, Y |
| Other | | N, Q, D, E |

Alternatively, conservative amino acids can be grouped as described in Lehninger (Biochemistry, Second Edition; Worth Publishers, Inc. NY:NY (1975), pp. 71-77) as set out in Table 2, immediately below.

TABLE 2
Conservative Substitutions II

| Side Chain | Characteristic | Amino Acid |
|---|---|---|
| Non-polar (hydrophobic) | Aliphatic: | A, L, I, V, P |
| | Aromatic | F, W |
| | Sulfur-containing | M |
| | Borderline | G |
| Uncharged-polar | Hydroxyl | S, T, Y |
| | Amides | N, Q |
| | Sulfhydryl | C |
| | Borderline | G |
| Positively Charged (Basic) | | K, R, H |
| Negatively Charged (Acidic) | | D, E |

Variants or derivatives can also have additional amino acid residues which arise from use of specific expression systems. For example, use of commercially available vectors that express a desired polypeptide as part of a glutathione-S-transferase (GST) fusion product provides the desired polypeptide having an additional glycine residue at position −1 after cleavage of the GST component from the desired polypeptide. Variants which result from expression in other vector systems are also contemplated, including those wherein histidine tags are incorporated into the amino acid sequence, generally at the carboxy and/or amino terminus of the sequence.

Deletion variants are also contemplated wherein one or more amino acid residues in a binding domain of this disclosure are removed. Deletions can be effected at one or both termini of the fusion protein, or from removal of one or more residues within the amino acid sequence.

In certain illustrative embodiments, fusion proteins of this disclosure are glycosylated, the pattern of glycosylation being dependent upon a variety of factors including the host cell in which the protein is expressed (if prepared in recombinant host cells) and the culture conditions. This disclosure also provides derivatives of fusion proteins. Derivatives include specific binding domain polypeptides bearing modifications other than insertion, deletion, or substitution of amino acid residues. In certain embodiments, the modifications are covalent in nature, and include for example, chemical bonding with polymers, lipids, other organic, and inorganic moieties. Derivatives of this disclosure may be prepared to increase circulating half-life of a specific binding domain polypeptide, or may be designed to improve targeting capacity for the polypeptide to desired cells, tissues, or organs.

This disclosure further embraces fusion proteins that are covalently modified or derivatized to include one or more water-soluble polymer attachments such as polyethylene glycol, polyoxyethylene glycol, or polypropylene glycol, as described U.S. Pat. Nos. 4,640,835; 4,496,689; 4,301,144; 4,670,417; 4,791,192 and 4,179,337. Still other useful polymers known in the art include monomethoxy-polyethylene glycol, dextran, cellulose, and other carbohydrate-based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of these polymers. Particularly preferred are polyethylene glycol (PEG)-derivatized proteins. Water-soluble polymers may be bonded at specific positions, for example at the amino terminus of the proteins and polypeptides according to this disclosure, or randomly attached to one or more side chains of the polypeptide. The use of PEG for improving therapeutic capacities is described in U.S. Pat. No. 6,133,426.

A particular embodiment of this disclosure is an immunoglobulin or an Fc fusion protein. Such a fusion protein can have a long half-life, e.g., several hours, a day or more, or even a week or more, especially if the Fc domain is capable of interacting with FcRn, the neonatal Fc receptor. The binding site for FcRn in an Fc domain is also the site at which the bacterial proteins A and G bind. The tight binding between these proteins can be used as a means to purify antibodies or fusion proteins of this disclosure by, for example, employing protein A or protein G affinity chromatography during protein purification.

Protein purification techniques are well known to those of skill in the art. These techniques involve, at one level, the crude fractionation of the polypeptide and non-polypeptide fractions. Further purification using chromatographic and electrophoretic techniques to achieve partial or complete purification (or purification to homogeneity) is frequently desired. Analytical methods particularly suited to the preparation of a pure fusion protein are ion-exchange chromatography; exclusion chromatography; polyacrylamide gel electrophoresis; and isoelectric focusing. Particularly efficient methods of purifying peptides are fast protein liquid chromatography and HPLC.

Certain aspects of the present disclosure concern the purification, and in particular embodiments, the substantial purification, of a fusion protein. The term “purified fusion protein” as used herein, is intended to refer to a composition, isolatable from other components, wherein the fusion protein is purified to any degree relative to its naturally obtainable state. A purified fusion protein therefore also refers to a fusion protein, free from the environment in which it may naturally occur.

Generally, “purified” will refer to a fusion protein composition that has been subjected to fractionation to remove various other components, and which composition substantially retains its expressed biological activity. Where the term “substantially purified” is used, this designation refers to a fusion binding protein composition in which the fusion protein forms the major component of the composition, such as constituting about 50%, about 60%, about 70%, about 80%, about 90%, about 95%, about 99% or more of the protein, by weight, in the composition.

Various methods for quantifying the degree of purification are known to those of skill in the art in light of the present disclosure. These include, for example, determining the specific binding activity of an active fraction, or assessing the amount of fusion protein in a fraction by SDS/PAGE analysis. A preferred method for assessing the purity of a protein fraction is to calculate the binding activity of the fraction, to compare it to the binding activity of the initial extract, and to thus calculate the degree of purification, herein assessed by a “-fold purification number.” The actual units used to represent the amount of binding activity will, of course, be dependent upon the particular assay technique chosen to follow the purification and whether or not the expressed fusion protein exhibits a detectable binding activity.

Various techniques suitable for use in protein purification are well known to those of skill in the art. These include, for example, precipitation with ammonium sulfate, PEG, antibodies and the like, or by heat denaturation, followed by centrifugation; chromatography steps such as ion exchange, gel filtration, reverse phase, hydroxylapatite, and affinity chromatography; isoelectric focusing; gel electrophoresis; and combinations of these and other techniques. As is generally known in the art, it is believed that the order of conducting the various purification steps may be changed, or that certain steps may be omitted, and still result in a suitable method for the preparation of a substantially purified protein.

There is no general requirement that the fusion protein always be provided in its most purified state. Indeed, it is contemplated that less substantially purified proteins will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in greater purification than the same technique utilizing a low pressure chromatography system. Methods exhibiting a lower degree of relative purification may have advantages in total recovery of protein product, or in maintaining binding activity of an expressed protein.

It is known that the migration of a polypeptide can vary, sometimes significantly, with different conditions of SDS/PAGE (Capaldi et al. (1977) Biochem. Biophys. Res. Comm. 76:425). It will therefore be appreciated that under differing electrophoresis conditions, the apparent molecular weights of purified or partially purified fusion protein expression products may vary.

### Polynucleotides, Expression Vectors, and Host Cells

This disclosure provides polynucleotides (isolated or purified or pure polynucleotides) encoding the multi-specific fusion protein of this disclosure, vectors (including cloning vectors and expression vectors) comprising such polynucleotides, and cells (e.g., host cells) transformed or transfected with a polynucleotide or vector according to this disclosure.

In certain embodiments, a polynucleotide (DNA or RNA) encoding a binding domain of this disclosure, or a multi-specific fusion protein containing one or more such binding domains is contemplated. Expression cassettes encoding multi-specific fusion protein constructs are provided in the examples appended hereto.

The present disclosure also relates to vectors that include a polynucleotide of this disclosure and, in particular, to recombinant expression constructs. In one embodiment, this disclosure contemplates a vector comprising a polynucleotide encoding a multi-specific fusion protein containing a CD72-ligand binding domain and a B-cell protein binding domain of this disclosure, along with other polynucleotide sequences that cause or facilitate transcription, translation, and processing of such multi-specific fusion protein-encoding sequences.

Appropriate cloning and expression vectors for use with prokaryotic and eukaryotic hosts are described, for example, in Sambrook et al., Molecular Cloning: A Laboratory Manual, Second Edition, Cold Spring Harbor, N.Y., (1989). Exemplary cloning/expression vectors include cloning vectors, shuttle vectors, and expression constructs, that may be based on plasmids, phagemids, phasmids, cosmids, viruses, artificial chromosomes, or any nucleic acid vehicle known in the art suitable for amplification, transfer, and/or expression of a polynucleotide contained therein

As used herein, “vector” means a nucleic acid molecule capable of transporting another nucleic acid to which it has been linked. Exemplary vectors include plasmids, yeast artificial chromosomes, and viral genomes. Certain vectors can autonomously replicate in a host cell, while other vectors can be integrated into the genome of a host cell and thereby are replicated with the host genome. In addition, certain vectors are referred to herein as “recombinant expression vectors” (or simply, “expression vectors”), which contain nucleic acid sequences that are operatively linked to an expression control sequence and, therefore, are capable of directing the expression of those sequences.

In certain embodiments, expression constructs are derived from plasmid vectors. Illustrative constructs include modified pNASS vector (Clontech, Palo Alto, Calif.), which has nucleic acid sequences encoding an ampicillin resistance gene, a polyadenylation signal and a T7 promoter site; pDEF38 and pNEF38 (CMC ICOS Biologics, Inc.), which have a CHEF1 promoter; and pD18 (Lonza), which has a CMV promoter. Other suitable mammalian expression vectors are well known (see, e.g., Ausubel et al., 1995; Sambrook et al., supra; see also, e.g., catalogs from Invitrogen, San Diego, Calif.; Novagen, Madison, Wis.; Pharmacia, Piscataway, N.J.). Useful constructs may be prepared that include a dihydrofolate reductase (DHFR)-encoding sequence under suitable regulatory control, for promoting enhanced production levels of the fusion proteins, which levels result from gene amplification following application of an appropriate selection agent (e.g., methotrexate).

Generally, recombinant expression vectors will include origins of replication and selectable markers permitting transformation of the host cell, and a promoter derived from a highly-expressed gene to direct transcription of a downstream structural sequence, as described above. A vector in operable linkage with a polynucleotide according to this disclosure yields a cloning or expression construct. Exemplary cloning/expression constructs contain at least one expression control element, e.g., a promoter, operably linked to a polynucleotide of this disclosure. Additional expression control elements, such as enhancers, factor-specific binding sites, terminators, and ribosome binding sites are also contemplated in the vectors and cloning/expression constructs according to this disclosure. The heterologous structural sequence of the polynucleotide according to this disclosure is assembled in appropriate phase with translation initiation and termination sequences. Thus, for example, the fusion protein-encoding nucleic acids as provided herein may be included in any one of a variety of expression vector constructs as a recombinant expression construct for expressing such a protein in a host cell.

The appropriate DNA sequence(s) may be inserted into a vector, for example, by a variety of procedures. In general, a DNA sequence is inserted into an appropriate restriction endonuclease cleavage site(s) by procedures known in the art. Standard techniques for cloning, DNA isolation, amplification and purification, for enzymatic reactions involving DNA ligase, DNA polymerase, restriction endonucleases and the like, and various separation techniques are contemplated. A number of standard techniques are described, for example, in Ausubel et al. (Current Protocols in Molecular Biology, Greene Publ. Assoc. Inc. & John Wiley & Sons, Inc., Boston, Mass., 1993); Sambrook et al. (Molecular Cloning, Second Ed., Cold Spring Harbor Laboratory, Plainview, N.Y., 1989); Maniatis et al. (Molecular Cloning, Cold Spring Harbor Laboratory, Plainview, N.Y., 1982); Glover (Ed.) (DNA Cloning Vol. I and II, IRL Press, Oxford, UK, 1985); Hames and Higgins (Eds.) (Nucleic Acid Hybridization, IRL Press, Oxford, UK, 1985); and elsewhere.

The DNA sequence in the expression vector is operatively linked to at least one appropriate expression control sequence (e.g., a constitutive promoter or a regulated promoter) to direct mRNA synthesis. Representative examples of such expression control sequences include promoters of eukaryotic cells or their viruses, as described above. Promoter regions can be selected from any desired gene using CAT (chloramphenicol transferase) vectors or other vectors with selectable markers. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art, and preparation of certain particularly preferred recombinant expression constructs comprising at least one promoter or regulated promoter operably linked to a nucleic acid encoding a protein or polypeptide according to this disclosure is described herein.

Variants of the polynucleotides of this disclosure are also contemplated. Variant polynucleotides are at least 90%, and preferably 95%, 99%, or 99.9% identical to one of the polynucleotides of defined sequence as described herein, or hybridize to one of those polynucleotides of defined sequence under stringent hybridization conditions of 0.015M sodium chloride, 0.0015M sodium citrate at about 65-68° C. or 0.015M sodium chloride, 0.0015M sodium citrate, and 50% formamide at about 42° C. The polynucleotide variants retain the capacity to encode a binding domain or fusion protein thereof having the functionality described herein.

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing are 0.015M sodium chloride, 0.0015M sodium citrate at about 65-68° C. or 0.015M sodium chloride, 0.0015M sodium citrate, and 50% formamide at about 42° C. (see Sambrook et al., Molecular Cloning: A Laboratory Manual, 2nd Ed., Cold Spring Harbor Laboratory, Cold Spring Harbor, N.Y., 1989).

More stringent conditions (such as higher temperature, lower ionic strength, higher formamide, or other denaturing agent) may also be used; however, the rate of hybridization will be affected. In instances wherein hybridization of deoxyoligonucleotides is concerned, additional exemplary stringent hybridization conditions include washing in 6×SSC, 0.05% sodium pyrophosphate at 37° C. (for 14-base oligonucleotides), 48° C. (for 17-base oligonucleotides), 55° C. (for 20-base oligonucleotides), and 60° C. (for 23-base oligonucleotides).

A further aspect of this disclosure provides a host cell transformed or transfected with, or otherwise containing, any of the polynucleotides or vector/expression constructs of this disclosure. The polynucleotides or cloning/expression constructs of this disclosure are introduced into suitable cells using any method known in the art, including transformation, transfection and transduction. Host cells include the cells of a subject undergoing ex vivo cell therapy including, for example, ex vivo gene therapy. Eukaryotic host cells contemplated as an aspect of this disclosure when harboring a polynucleotide, vector, or protein according to this disclosure include, in addition to a subject's own cells (e.g., a human patient's own cells), VERO cells, HeLa cells, Chinese hamster ovary (CHO) cell lines (including modified CHO cells capable of modifying the glycosylation pattern of expressed multivalent binding molecules, see US Patent Application Publication No. 2003/0115614), COS cells (such as COS-7), W138, BHK, HepG2, 3T3, RIN, MDCK, A549, PC12, K562, HEK293 cells, HepG2 cells, N cells, 3T3 cells, Spodoptera frugiperda cells (e.g., Sf9 cells), Saccharomyces cerevisiae cells, and any other eukaryotic cell known in the art to be useful in expressing, and optionally isolating, a protein or peptide according to this disclosure. Also contemplated are prokaryotic cells, including Escherichia coli, Bacillus subtilis, Salmonella typhimurium, a Streptomycete, or any prokaryotic cell known in the art to be suitable for expressing, and optionally isolating, a protein or peptide according to this disclosure. In isolating protein or peptide from prokaryotic cells, in particular, it is contemplated that techniques known in the art for extracting protein from inclusion bodies may be used. The selection of an appropriate host is within the scope of those skilled in the art from the teachings herein. Host cells that glycosylate the fusion proteins of this disclosure are contemplated.

The term “recombinant host cell” (or simply “host cell”) refers to a cell containing a recombinant expression vector. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

Recombinant host cells can be cultured in a conventional nutrient medium modified as appropriate for activating promoters, selecting transformants, or amplifying particular genes. The culture conditions for particular host cells selected for expression, such as temperature, pH and the like, will be readily apparent to the ordinarily skilled artisan. Various mammalian cell culture systems can also be employed to express recombinant protein. Examples of mammalian expression systems include the COS-7 lines of monkey kidney fibroblasts, described by Gluzman (1981) Cell 23:175, and other cell lines capable of expressing a compatible vector, for example, the C127, 3T3, CHO, HeLa and BHK cell lines. Mammalian expression vectors will comprise an origin of replication, a suitable promoter and, optionally, enhancer, and also any necessary ribosome binding sites, polyadenylation site, splice donor and acceptor sites, transcriptional termination sequences, and 5′-flanking nontranscribed sequences, for example, as described herein regarding the preparation of multivalent binding protein expression constructs. DNA sequences derived from the SV40 splice, and polyadenylation sites may be used to provide the required nontranscribed genetic elements. Introduction of the construct into the host cell can be effected by a variety of methods with which those skilled in the art will be familiar, including calcium phosphate transfection, DEAE-Dextran-mediated transfection, or electroporation (Davis et al. (1986) Basic Methods in Molecular Biology).

In one embodiment, a host cell is transduced by a recombinant viral construct directing the expression of a protein or polypeptide according to this disclosure. The transduced host cell produces viral particles containing expressed protein or polypeptide derived from portions of a host cell membrane incorporated by the viral particles during viral budding.

### Compositions and Methods of Use

To treat human or non-human mammals suffering a disease state associated with B-cell dysregulation, a multi-specific fusion protein of this disclosure is administered to the subject in an amount that is effective to ameliorate symptoms of the disease state following a course of one or more administrations. Being polypeptides, the multi-specific fusion proteins of this disclosure can be suspended or dissolved in a pharmaceutically acceptable diluent, optionally including a stabilizer of other pharmaceutically acceptable excipients, which can be used for intravenous administration by injection or infusion, as more fully discussed below.

A pharmaceutically effective dose is that dose required to prevent, inhibit the occurrence of, or treat (alleviate a symptom to some extent, preferably all symptoms of) a disease state. The pharmaceutically effective dose depends on the type of disease, the composition used, the route of administration, the type of subject being treated, the physical characteristics of the specific subject under consideration for treatment, concurrent medication, and other factors that those skilled in the medical arts will recognize. For example, an amount between 0.1 mg/kg and 100 mg/kg body weight (which can be administered as a single dose, or in multiple doses given hourly, daily, weekly, monthly, or any combination thereof that is an appropriate interval) of active ingredient may be administered depending on the potency of a binding domain polypeptide or multi-specific protein fusion of this disclosure.

In certain aspects, compositions of fusion proteins are provided by this disclosure. Pharmaceutical compositions of this disclosure generally comprise one or more type of binding domain or fusion protein in combination with a pharmaceutically acceptable carrier, excipient, or diluent. Such carriers will be nontoxic to recipients at the dosages and concentrations employed. Pharmaceutically acceptable carriers for therapeutic use are well known in the pharmaceutical art, and are described, for example, in Remington's Pharmaceutical Sciences, Mack Publishing Co. (A.R. Gennaro (Ed.) 1985). For example, sterile saline and phosphate buffered saline at physiological pH may be used. Preservatives, stabilizers, dyes and the like may be provided in the pharmaceutical composition. For example, sodium benzoate, sorbic acid, or esters of p-hydroxybenzoic acid may be added as preservatives. Id. at 1449. In addition, antioxidants and suspending agents may be used. Id. The compounds of the present invention may be used in either the free base or salt forms, with both forms being considered as being within the scope of the present invention.

Pharmaceutical compositions may also contain diluents such as buffers; antioxidants such as ascorbic acid, low molecular weight (less than about 10 residues) polypeptides, proteins, amino acids, carbohydrates (e.g., glucose, sucrose, or dextrins), chelating agents (e.g., EDTA), glutathione or other stabilizers or excipients. Neutral buffered saline or saline mixed with nonspecific serum albumin are exemplary appropriate diluents. Preferably, product is formulated as a lyophilizate using appropriate excipient solutions as diluents.

CD19 overexpression has been implicated in systemic sclerosis (Sato et al., J. Immun 165:6635, 2000), and Saito et al., (J. Clin. Invest. 109:1453, 2002) concluded that chronic B-cell activation in mice resulting from augmented CD19 signaling leads to skin sclerosis and autoimmunity, possibly through overproduction of IL-6. Defects in CD19 expression impair B cell signaling through the B cell receptor (BCR) and can lead to hypogammaglobulinemia in which the response of mature B cells to antigenic stimulation is defective (Van Zelm et al., New Eng. J. Med. 354:1901-1912, 2006). These defects can also lead to primary antibody deficiency (Van Zelm, supra). B cell disorders which may benefit from modulation of CD19 activity include B cell cancers (for example, B-cell lymphomas, B-cell leukemias, B-cell lymphomas), diseases characterized by autoantibody production or diseases characterized by inappropriate stimulation of T-cells, such as by inappropriate B-cell antigen binding to T-cells or by other pathways involving B-cells.

Research and drug development has occurred based on the concept that B-cell lineage-specific cell surface molecules such as CD37 or CD20 can themselves be targets for antibodies that would bind to, and mediate destruction of, cancerous and autoimmune disease-causing B-cells that have CD37 on their surface. One antibody to CD37 has been labeled with ¹³¹I and tested in clinical trials for therapy of NHL. See Press et al., J. Clin. Oncol. 7:1027 (1989); Bernstein et al., Cancer Res. (Suppl.) 50:1017 (1990); Press et al., Front. Radiat. Ther. Oncol. 24:204 (1990); Press et al., Adv. Exp. Med. Biol. 303:91 (1991) and Brown et al., Nucl. Med. Biol. 24:657 (1997). The antibody, MB-1, is a murine IgG1 monoclonal antibody that lacks Fc effector functions such as antibody-dependent cellular cytotoxicity (ADCC) and MB-1 did not inhibit tumor growth in an in vivo xenograft model unless it had been labeled with an isotope (Buchsbaum et al. (1992) Cancer Res. 52:6476). Favorable biodistribution of ¹³¹I-MB-1 was seen in lymphoma patients who had lower tumor burdens (<1 kg) and therapy of these patients resulted in complete tumor remissions lasting from 4 to 11 months (Press et al., 1989 and Bernstein et al. 1990, supra). In addition, an immunoconjugate composed of the drug adriamycin linked to G28-1, another anti-CD37 antibody, has been evaluated in mice and showed effects through internalization and intracellular release of the drug (see Braslawsky et al. (1991) Cancer Immunol. Immunother. 33:367).

CD20 is expressed by malignant cells of B-cell origin, including B-cell lymphoma and chronic lymphocytic leukemia (CLL). CD20 is not expressed by malignancies of pre-B-cells, such as acute lymphoblastic leukemia. CD20 is therefore a good target for therapy of B-cell lymphoma, CLL, and other diseases in which B-cells are involved in the disease etiology. Other B-cell disorders include autoimmune diseases in which autoantibodies are produced during the differentiation of B-cells into plasma cells. Because normal mature B-cells also express CD20, normal B-cells are depleted by anti-CD20 antibody therapy (Reff et al., Blood 1994, 83:435-445). After treatment is completed, however, normal B-cells can be regenerated from CD20 negative B-cell precursors; therefore, patients treated with anti-CD20 therapy do not experience significant immunosuppression.

Anti-CD20 monoclonal antibodies affect the viability and growth of B-cells. (Clark et al., Proc. Natl. Acad. Sci. USA 1986, 83:4494-98). Extensive cross-linking of CD20 can induce apoptosis in B lymphoma cell lines (Shan et al., Blood 1998, 91:1644-52), and cross-linking of CD20 on the cell surface has been reported to increase the magnitude and enhance the kinetics of signal transduction, for example, as detected by measuring tyrosine phosphorylation of cellular substrates. (Deans et al., J. Immunol. 1993, 146:846-53). Therefore, in addition to cellular depletion by complement and ADCC mechanisms, Fc-receptor binding by CD20 monoclonal antibodies in vivo may promote apoptosis of malignant B-cells by CD20 cross-linking, consistent with the theory that effectiveness of CD20 therapy of human lymphoma in a SCID mouse model may be dependent upon Fc-receptor binding by the CD20 monoclonal antibody (Funakoshi et al., J. Immunotherapy 1996, 19:93-101). The presence of multiple membrane spanning domains in the CD20 polypeptide (Einfeld et al., EMBO J. 1988, 7:711-17; Stamenkovic et al., J. Exp. Med. 1988, 167:1975-80; Tedder et al., J. Immunol. 1988, 141:4388-4394), prevent CD20 internalization after antibody binding, and this was recognized as an important feature for therapy of B-cell malignancies when a murine CD20 monoclonal antibody, 1F5, was injected into patients with B-cell lymphoma, resulting in significant depletion of malignant cells and partial clinical responses (Press et al., Blood 1987, 69:584-91).

The FCRL1-6 proteins are likely involved in similar B-cell disorders as those associated with CD20, such as B-cell lymphomas and rheumatoid arthritis.

Defects in CD79a are a cause of non-Bruton type agammaglobulinemia, which is an immunodeficiency disease and results in developmental defects in the maturation pathway of B-cells. CD79a positive cells have also been found in lymphomas and leukemias, including precursor B-acute lymphoblastic leukemia (pre-B-ALL), T-cell acute lymphoblastic leukemia (T-ALL), acute lymphocytic leukemia, acute myeloid leukemia, biphenotypic acute leukemia (BAL) (Kozlov et al., Cancer Genet. Cytogenet. 2005 November; 163(1):62-7), diffuse large B-cell lymphoma, precursor B-cell lymphoblastic lymphoma, non-Hodgkin lymphoma, classical Hodgkin's lymphoma, mucosa-associated lymphoid tissue (MALT) lymphoma, and anaplastic large cell lymphoma (ALCL) commonly seen in HIV patients.

CD79b positive cells have also been found in lymphomas and leukemias, including Non-Hodgkin's lymphoma, chronic lymphocytic leukemia (Cajiao et al., Am. J. Hematol. 2007 82(8):712-20), lymphoplasmacytic lymphoma/Waldenström macroglobulinemia (Konoplev et al., Am. J. Clin. Pathol. 2005 September; 124(3):414-20), chronic lymphocytic leukemia/small lymphocytic lymphoma (CLL/SLL), and mantle cell lymphomas (MCL) (D'Arena et al., Am. J. Hematol. 2000 64(4):275-81. It has also been suggested that low expression of CD79b may lead to decreased surface Ig (sIg) expression and B cell chronic lymphocytic leukemia (B-CLL) (Minuzzo et al., Br. J. Haematol. 2005 September; 130(6):878-89). Studies have shown that a CD79b variant, DeltaCD79b, may be transcribed in CLL B cells, and inhibits apoptosis of these cells and aberrant expression of neoplastic B cells (Cragg et al., Blood. 2002 100(9):3068-76).

Thus, agents comprising binding domains of this disclosure are useful in treating B-cell related hyperproliferative, inflammatory, or autoimmune diseases disclosed herein.

B-cell cancers include B-cell lymphomas (such as various forms of Hodgkin's disease, non-Hodgkins lymphoma (NHL) or central nervous system lymphomas), leukemias (such as acute lymphoblastic leukemia (ALL), chronic lymphocytic leukemia (CLL), Hairy cell leukemia and chronic myoblastic leukemia), and myelomas (such as multiple myeloma). Additional B cell cancers include small lymphocytic lymphoma, B-cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B-cell lymphoma of mucosa-associated (MALT) lymphoid tissue, nodal marginal zone B-cell lymphoma, follicular lymphoma, mantle cell lymphoma, diffuse large B-cell lymphoma, mediastinal (thymic) large B-cell lymphoma, intravascular large B-cell lymphoma, primary effusion lymphoma, Burkitt lymphoma/leukemia, B-cell proliferations of uncertain malignant potential, lymphomatoid granulomatosis, and post-transplant lymphoproliferative disorder.

Disorders characterized by autoantibody production are often considered autoimmune diseases. Autoimmune diseases include arthritis, rheumatoid arthritis, juvenile rheumatoid arthritis, osteoarthritis, polychondritis, psoriatic arthritis, psoriasis, dermatitis, polymyositis/dermatomyositis, inclusion body myositis, inflammatory myositis, toxic epidermal necrolysis, systemic scleroderma and sclerosis, CREST syndrome, responses associated with inflammatory bowel disease, Crohn's disease, ulcerative colitis, respiratory distress syndrome, adult respiratory distress syndrome (ARDS), meningitis, encephalitis, uveitis, colitis, glomerulonephritis, allergic conditions, eczema, asthma, conditions involving infiltration of T cells and chronic inflammatory responses, atherosclerosis, autoimmune myocarditis, leukocyte adhesion deficiency, systemic lupus erythematosus (SLE), subacute cutaneous lupus erythematosus, discoid lupus, lupus myelitis, lupus cerebritis, juvenile onset diabetes, multiple sclerosis, allergic encephalomyelitis, neuromyelitis optica, rheumatic fever, Sydenham's chorea, immune responses associated with acute and delayed hypersensitivity mediated by cytokines and T-lymphocytes, tuberculosis, sarcoidosis, granulomatosis including Wegener's granulomatosis and Churg-Strauss disease, agranulocytosis, vasculitis (including hypersensitivity vasculitis/angiitis, ANCA and rheumatoid vasculitis), aplastic anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, central nervous system (CNS) inflammatory disorders, multiple organ injury syndrome, mysathenia gravis, antigen-antibody complex mediated diseases, anti-glomerular basement membrane disease, anti-phospholipid antibody syndrome, allergic neuritis, Behcet disease, Castleman's syndrome, Goodpasture's syndrome, Lambert-Eaton Myasthenic Syndrome, Reynaud's syndrome, Sjorgen's syndrome, Stevens-Johnson syndrome, solid organ transplant rejection, graft versus host disease (GVHD), pemphigoid bullous, pemphigus, autoimmune polyendocrinopathies, seronegative spondyloarthropathies, Reiter's disease, stiff-man syndrome, giant cell arteritis, immune complex nephritis, IgA nephropathy, IgM polyneuropathies or IgM mediated neuropathy, idiopathic thrombocytopenic purpura (ITP), thrombotic throbocytopenic purpura (TTP), Henoch-Schonlein purpura, autoimmune thrombocytopenia, autoimmune disease of the testis and ovary including autoimmune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes (also referred to as insulin-dependent diabetes mellitus (IDDM)) and Sheehan's syndrome; autoimmune hepatitis, lymphoid interstitial pneumonitis (HIV), bronchiolitis obliterans (non-transplant) vs NSIP, Guillain-Barre' Syndrome, large vessel vasculitis (including polymyalgia rheumatica and giant cell (Takayasu's) arteritis), medium vessel vasculitis (including Kawasaki's disease and polyarteritis nodosa), polyarteritis nodosa (PAN) ankylosing spondylitis, Berger's disease (IgA nephropathy), rapidly progressive glomerulonephritis, primary biliary cirrhosis, Celiac sprue (gluten enteropathy), cryoglobulinemia, cryoglobulinemia associated with hepatitis, amyotrophic lateral sclerosis (ALS), coronary artery disease, familial Mediterranean fever, microscopic polyangiitis, Cogan's syndrome, Whiskott-Aldrich syndrome and thromboangiitis obliterans.

Also contemplated is the administration of multi-specific fusion protein compositions of this disclosure in combination with a second agent. A second agent may be one accepted in the art as a standard treatment for a particular disease state, such as inflammation, autoimmunity, and cancer. Exemplary second agents contemplated include cytokines, growth factors, steroids, NSAIDs, DMARDs, chemotherapeutics, radiotherapeutics, or other active and ancillary agents, or any combination thereof.

“Pharmaceutically acceptable salt” refers to a salt of a binding domain polypeptide or fusion protein of this disclosure that is pharmaceutically acceptable and that possesses the desired pharmacological activity of the parent compound. Such salts include the following: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, or the like.

In particular illustrative embodiments, a polypeptide or fusion protein of this disclosure is administered intravenously by, for example, bolus injection or infusion. Routes of administration in addition to intravenous include oral, topical, parenteral (e.g., sublingually or buccally), sublingual, rectal, vaginal, and intranasal. The term parenteral as used herein includes subcutaneous injections, intravenous, intramuscular, intrasternal, intracavernous, intrathecal, intrameatal, intraurethral injection or infusion techniques. The pharmaceutical composition is formulated so as to allow the active ingredients contained therein to be bioavailable upon administration of the composition to a patient. Compositions that will be administered to a patient take the form of one or more dosage units, where for example, a tablet may be a single dosage unit, and a container of one or more compounds of this disclosure in aerosol form may hold a plurality of dosage units. In a composition intended to be administered by injection, one or more of a surfactant, preservative, wetting agent, dispersing agent, suspending agent, buffer, stabilizer, isotonic agent, or any combination thereof may optionally be included.

For oral administration, an excipient and/or binder may be present, such as sucrose, kaolin, glycerin, starch dextrans, cyclodextrins, sodium alginate, ethyl cellulose, and carboxy methylcellulose. Sweetening agents, preservatives, dye/colorant, flavor enhancer, or any combination thereof may optionally be present. A coating shell may optionally be used.

For nucleic acid-based formulations, or for formulations comprising expression products according to this disclosure, about 0.01 μg/kg to about 100 mg/kg body weight can be administered, for example, by intradermal, subcutaneous, intramuscular, or intravenous routes, or by any route known in the art to be suitable under a given set of circumstances. A preferred dosage, for example, is about 1 μg/kg to about 20 mg/kg, with about 5 μg/kg to about 10 mg/kg particularly preferred. It will be evident to those skilled in the art that the number and frequency of administration will be dependent upon the response of the host.

The pharmaceutical compositions of this disclosure may be in any form that allows for administration to a patient, such as, for example, in the form of a solid, liquid, or gas (aerosol). The composition may be in the form of a liquid, e.g., an elixir, syrup, solution, emulsion or suspension, for administration by any route described herein.

A liquid pharmaceutical composition as used herein, whether in the form of a solution, suspension or other like form, may include one or more of the following components: sterile diluents such as water for injection, saline solution (e.g., physiological saline), Ringer's solution, isotonic sodium chloride, fixed oils such as synthetic mono- or digylcerides that may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; buffers such as acetates, citrates or phosphates; chelating agents such as ethylenediaminetetraacetic acid; and agents for the adjustment of tonicity such as sodium, chloride, or dextrose. The parenteral preparation can be enclosed in ampoules, disposable syringes or multiple dose vials made of glass or plastic. Physiological saline is a preferred additive. An injectable pharmaceutical composition is preferably sterile.

It may also be desirable to include other components in the preparation, such as delivery vehicles including aluminum salts, water-in-oil emulsions, biodegradable oil vehicles, oil-in-water emulsions, biodegradable microcapsules, and liposomes. Examples of adjuvants for use in such vehicles include N-acetylmuramyl-L-alanine-D-isoglutamine (MDP), lipopolysaccharides (LPS), glucan, IL-12, GM-CSF, γ-interferon, and IL-15.

While any suitable carrier known to those of ordinary skill in the art may be employed in the pharmaceutical compositions of this disclosure, the type of carrier will vary depending on the mode of administration and whether a sustained release is desired. For parenteral administration, the carrier may comprise water, saline, alcohol, a fat, a wax, a buffer, or any combination thereof. For oral administration, any of the above carriers or a solid carrier, such as mannitol, lactose, starch, magnesium stearate, sodium saccharine, talcum, cellulose, glucose, sucrose, magnesium carbonate, or any combination thereof, may be employed.

This disclosure contemplates a dosage unit comprising a pharmaceutical composition of this disclosure. Such dosage units include, for example, a single-dose or a multi-dose vial or syringe, including a two-compartment vial or syringe, one comprising the pharmaceutical composition of this disclosure in lyophilized form and the other a diluent for reconstitution. A multi-dose dosage unit can also be, e.g., a bag or tube for connection to an intravenous infusion device. This disclosure also contemplates a kit comprising a pharmaceutical composition in a unit dose or multi-dose container, e.g., a vial, and a set of instructions for administering the composition to patients suffering a disorder as described herein.

All U.S. patents, U.S. patent application publications, U.S. patent applications, foreign patents, foreign patent applications, non-patent publications, tables, sequences, webpages, or the like referred to in this specification, are incorporated herein by reference, in their entirety. The following examples are intended to illustrate, but not limit, this disclosure.

### EXAMPLES

### Xceptor Sequences

The amino acid sequences of exemplary multi-specific fusion proteins having a CD72 ectodomain and a CD19, CD37 or CD79b binding domain are provided in SEQ ID NOS: 9, 11, 13, 15, 17, 178, 180, 182 and 184 with the corresponding nucleic acid expression cassettes being provided in SEQ ID NO: 8, 10, 12, 14, 16, 177, 179, 181 and 183 respectively. Note the mature proteins will lack the signal peptide sequence found in SEQ ID NOS: 9, 11, 13, 15, 17, 178, 180, 182 and 184.

Multi-specific fusion proteins having a CD19 or CD37 binding domain at the amino-terminus and a CD72 ectodomain at the carboxy terminus are referred to herein as X1972 and X3772, respectively. The different versions of X3772 are referred to as X3772.1 (version 1), X3772.2 (version 2), and X3772.3 (version 3), which differ from wild-type by changes in the back-end linker. Multi-specific fusion proteins having a CD79B binding domain at the amino-terminus and a CD72 ectodomain at the carboxy terminus are referred to herein as X7972. The different versions of X7972 are referred to as X7972.1 (version 1), X7972.2 (version 2), and X7972.3 (version 3).

The activity of various Xceptor fusion proteins described herein was tested as described below. Abbreviations used in the following examples include the following terms: PBS-T: PBS, pH 7.2-7.4 and 0.1% Tween®20; Working buffer: PBS-T with 1% BSA; Blocking buffer: PBS-T with 3% BSA.

### Example 1

### Constructing Reagents and Xceptors

Xceptor fusion proteins comprising a CD-72 ligand binding domain (CD72 ectodomain) and either a CD19 binding domain or a CD37 binding domain were constructed substantially as follows.

### Cloning of CD72 ECD

CD72 ECD was cloned from human lymph node quick-clone cDNA (from Invitrogen) using CD72Full_F (ggtaaacgtacgcgctatctgcaggtgtctcagcagctc; SEQ ID NO: 167) and CD72Full_R (aggtactctagactaatctggaaacctgaaagctgtcatc; SEQ ID NO: 168) oligos. The fragment was inserted into TOPO vector (pCR4-TOPO from Invitrogen) and verified by DNA sequencing. The amino acid sequence of CD72 ECD with stalk is shown in SEQ ID NO:2, with the corresponding nucleic acid sequence being shown in SEQ ID NO: 3. Next, the fragment was cut from the TOPO vector using BsiWI and XbaI restriction sites and inserted into the PD18 vector together with the mouse Fc tail (CH2CH3 IgG2a) using the HindIII and BsiWI restriction sites to give the mIg CD72 (also known as mouse Ig CD72 ECD) construct. The sequence was again confirmed by sequencing. The DNA and protein sequences of mIg CD72 are shown in SEQ ID NO:4 and 5, respectively. HuIg CD72 was also constructed and its DNA and protein sequences are shown in SEQ ID NO:6 and 7, respectively.

### Construction of X3772 Xceptor

Using the pD18F (gtctatataagcagagctctctggc; SEQ ID NO: 169) and BsiWICH3_R (ctgcagatagcgcgtacgcttacccggagacagggagaggct; SEQ ID NO: 170) oligonucleotides as primers in a PCR reaction, the CD37 binding domain and Fc tail were cloned out from TRU016 DNA (anti-CD37 SMIP containing the G28-1 binding domain). This is the first fragment. Next, the CD72 ECD was also cloned out from the mIg CD72 DNA using CD72_BSIWI_F (tctccgggtaagcgtacgcgctatctgcaggtgtctc; SEQ ID NO: 171) and CD72_NotI_R (gatcttcgaggcggccgctctagactaatctggaaacctgaaagc; SEQ ID NO: 172). This is the second fragment. The first fragment was digested with HindIII and BsiWI and the second fragment was digested with BsiWI and NotI. These two fragments were then ligated into the pD28 vector that had been cut with HIndIII and NotI to give the X3772 Xceptor molecule. The DNA sequence was confirmed by sequencing and is shown in SEQ ID NO:10.

### Construction of X3772 Xceptor Variants

Three variants of the X3772 were made. These variants have shorter stalks or linkers that joined to the CD72 ECD. Version 1 has one single strand (35 amino acids), version 2 has half a strand (25 amino acids) while version 3 has the Linker 126. For version 1, the oligonucleotides CD72stalklF (ccgggtaagcgtacgcaaagtgaggagcaacagaggagg; SEQ ID NO: 173) and BsrG1_R (gggcagggtgtacacctgtggttctcggggc; SEQ ID NO: 174) were used to amplify the CD72 ECD fragment with one strand stalk. This fragment was digested with BsiWI and BsrG1 and religated into the anti-CD37XCD72 Xceptor vector that had been cut with the same two enzymes. These steps were repeated for the construction of the other two versions of the Xceptor except that the oligonucleotide pair of CD72stalk 2F/BsrGI_R (ggtaagcgtacggagcagaagctgagcaacatggag; SEQ ID NO: 175) and CD72NKG2AF/BsrGI_R (ggtaagcgtacgcagaggcacaacaattatccctgaatacaagaactcagaaagcacgtcattctggccattgtccgteggg-atggataatgc; SEQ ID NO: 176) were used for versions 2 and 3, respectively. The sequences of these variants have been confirmed by DNA sequencing and are shown in SEQ ID NOS:, 12, 14 and 16.

### Construction of X1972 Xceptor

Using the PD18F and BsiWICH3_R oligonucleotides as primers in a PCR reaction (sequences provided above), the CD19 binding domain and Fc tail were cloned out from M0018 DNA (anti-CD19 SMIP containing the HD37 binding domain). This is the first fragment. Next, the CD72 ECD was also cloned out from the mIg CD72 DNA using CD72_BSIWI_F and CD72_NotI_R. This is the second fragment. The first fragment was digested with HindIII and BsiWI and the second fragment was digested with BsiWI and NotI. These two fragments were ligated into the pD28 vector that had been cut with HIndIII and NotI to give the X1972 Xceptor molecule. The DNA sequence was confirmed by sequencing and is shown in SEQ ID NO:8.

### Construction of X7972 Xceptor

The anti-CD37xCD72 Xceptor construct (described above) was used as a template to build an anti-CD79BxCD72 Xceptor. First, the anti-CD79B scFv (PC2C) was cut from the CD79B SMIP vector (M0077) to release the HindIII/BsrGI fragment which was ligated into the anti-CD37xCD72 Xceptor vector that had been cut with HindIII and BsrGI restriction enzymes. This construct with the wt CD72 stalk as the scorpion linker is referred to as construct Q0011. The DNA and protein sequences were confirmed by sequencing and are provided in SEQ ID NO: 177 and 178, respectively.

The scorpion linker was subsequently engineered with shorter linkers referred to as variant 1, variant 2 and variant 3. The DNA sequences for these constructs (referred to as X7972.1, X7972.2 and X7972.3) are provided in SEQ ID NO: 179, 181 and 183, with the corresponding amino acid sequences being provided in SEQ ID NO: 180, 182 and 184, respectively.

### Example 2

### Characterization of Xceptor X7972

The DNA constructs encoding the X7972.1, X7972.2 and X7972.3 molecules were each separately transfected into HEK293 cells for 7 days. Cell culture supernatants were purified from HEK293 culture supernatants by Protein A affinity chromatography. Using dPBS, a 50 mL rProtein A FF sepharose column (GE Healthcare rProtein A Sepharose FF) was equilibrated at 5.0 mls/min (150 cm/hr) for 1.5 column volumes (CV). The culture supernatant was loaded to the rProtein A Sepharose FF column at a flow rate of 1.7 mls/min using the AKTA Explorer 100 Air (GE healthcare AKTA Explorer 100 Air), capturing the recombinant proteins. The column was washed with dPBS for 5 CV, then 1.0 M NaCl, 20 mM Sodium Phosphate, pH 6.0, and then with 25 mM NaCl, 25 mM NaOAc, pH 5.0. These washing steps removed nonspecifically bound CHO host cell proteins from the rProtein A column that contribute to product precipitation after elution.

X7972.1, X7972.2 and X7972.3 protein was subjected to reducing and non-reducing SDS-PAGE analysis on 4-20% Novex Tris-glycine gels (Invitrogen, San Diego, Calif.). Samples were loaded using Novex Tris-glycine SDS sample buffer (2×) under reducing (addition of 1/10 volume NuPAGE sample reducing agent) or non-reducing conditions after heating at 95° C. for 3 minutes, followed by electrophoresis at 150V for 90 minutes. Electrophoresis was performed using 1X Novex Tris-Glycine SDS Running Buffer (Invitrogen). Gels were stained after electrophoresis in Coomassie SDS PAGE R-250 stain for 30 minutes with agitation, and destained for at least one hour.

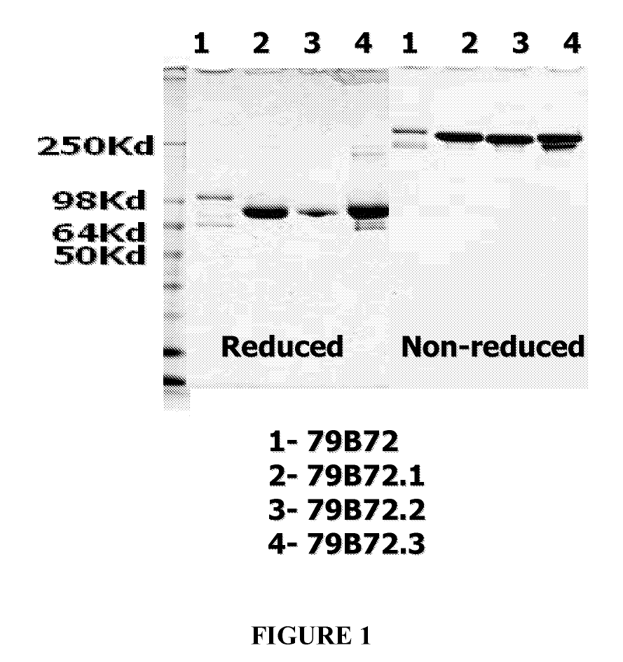

FIG. 1 shows the SDS-PAGE characterization of the X7972 Xceptor molecules showing that all of the variant X7972.1, X7972.2 and X7972.3 proteins can be produced, although the wt CD72 linker is somewhat more susceptible to degredation in 293 cells while the molecules containing a variant linker appeared stable in 293 cells.

### Example 3

### Xceptor Binding to CD79b or CD100 by ELISA

CD79b and/or CD100 binding activity was examined for Xceptors X7972, X7972.1, X7972.2 and X7972.3 substantially as follows.

Added to each well of a 96-well plate was 100 μl CD79b AFH (affinity flag his tag) or CD100-mIg fusion from a 2 μg/ml solution in PBS, pH 7.2-7.4. The plate was covered, and incubated overnight at 4° C. After washing four times with PBS-T, 250 μl Blocking buffer was added to each well, the plate was covered, and incubated at room temperature for 2 hours (or at 4° C. overnight). After washing the plate three times with PBS-T, added in duplicate wells to the CD79b AFH coated plate was 100 μl/well Xceptors X7972, X7972.1, X7972.2 and X7972.3, huIgCD72, anti-CD79B SMIP, and negative controls human IgG, each serially diluted three-fold in Working buffer starting at 300 ng/ml, the plate was covered, and incubated at room temperature for about 1 to 2 hours. The CD79b plates were washed three times with PBS-T, 100 μl per well Quantablue NS/K Fluorgenic substrate (Pierce Chemical Co., Rockford, Ill.) was added, incubated for 5 minutes and then read at on a Spectra Max Gemini XS plate reader (Molecular Devices Corp., Sunnyvale, Calif.). The samples were excited at 325 nm and emission at 420 nm was monitored (results are expressed as fluorescence intensity, FI). The CD100 plates were washed five times with PBS-T, 100 μl per well horse radish peroxidase-conjugated streptavidin (Jackson ImmunoResearch, West Grove, Pa.) diluted 1:1,000 in Working buffer was added, the plate was covered, and incubated at room temperature for 30 minutes. After washing the plate six times with PBS-T, 100 μl per well 3,3,5,5-tetramentylbenzidine (TMB) substrate solution (Pierce, Rockford, Ill.) was added for about 3 to 5 minutes and then the reaction was stopped with 50 μl Stop buffer (1N H₂SO₄) per well. The absorbance of each well was read at 450 nm.

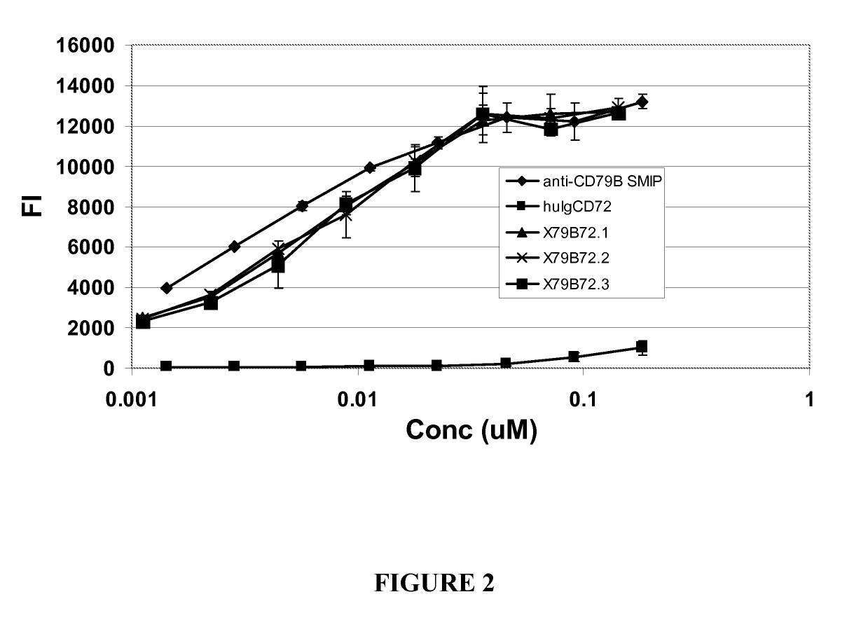

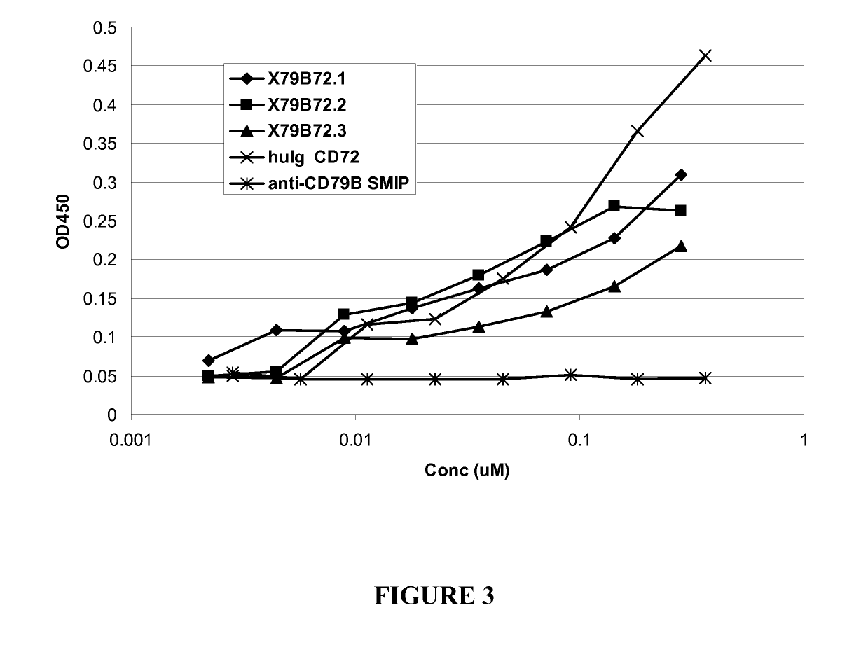

FIG. 2 shows that X7972.1, X7972.2 and X7972.3 all bound to CD79B AFH, with the binding being as good as anti-CD79B SMIP. FIG. 3 shows the results of binding to CD100, wherein all the molecules bound to CD100, although X7972.3 seemed to bind slightly less well than X7972.1 or X7972.2.

### Example 4

### Xceptor Dual Ligand Binding by ELISA

Concurrent binding to CD79b and CD100 was examined for Xceptors X7972.1 and X7972.2, substantially as follows.

Added to each well of a 96-well plate was 100 μl CD79b AFH solution (5 μg/ml in PBS, pH 7.2-7.4). The plate was covered, and incubated overnight at 4° C. After washing four times with PBS-T, 250 μl Blocking buffer was added to each well, the plate was covered, and incubated at room temperature for 2 hours (or at 4° C. overnight). After washing the plate three times with PBS-T, added in duplicate wells to the CD79B AFH coated plate were 100 μl/well X7972.1, X7972.2, huIgCD72 and anti-CD79B SMIP samples serially diluted three-fold in Working buffer starting at 300 ng/ml. Negative controls included human CD72-huIg, CD79b SMIP (M0077), and Working buffer only. The plate was covered and incubated at room temperature for 1.5 hours. After washing the plate five times with PBS-T, 100 μl per well CD100 AFH to 2 ng/ml in Working buffer was added, the plate was covered, and incubated at room temperature for 1.5 hr. After washing the plate five times with PBS-T, 100 μl per well horse radish peroxidase-conjugated streptavidin (Jackson ImmunoResearch, West Grove, Pa.) diluted 1:1000 in Working buffer was added, the plate was covered, and incubated at room temperature for 30 minutes. After washing the plate six times with PBS-T, 100 μl per well 3,3,5,5-tetramentylbenzidine (TMB) substrate solution (Pierce, Rockford, Ill.) was added for 3-5 minutes and then the reaction was stopped with 50 μl Stop buffer (1N H₂SO₄) per well. The absorbance of each well was read at 450 nm.

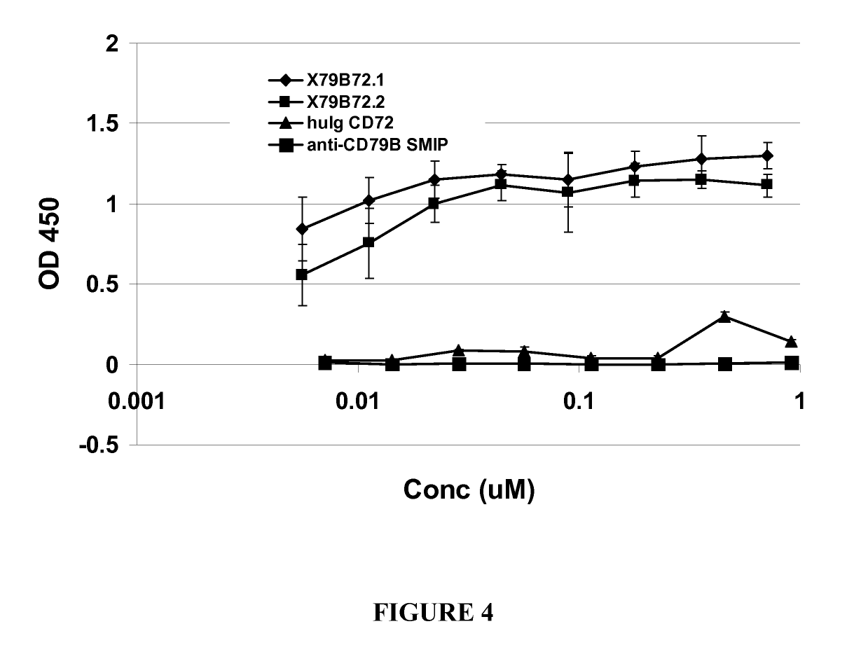

As shown in FIG. 4, both X7972.1 and X7972.2 could simultaneously bind CD79b and CD100.

### Example 5

### Xceptor Binding to BJAB and Ramos B-Cells

Binding of Xceptors X1972 and X3772 to the EBV negative Burkitt's Lymphoma cell line BJAB was compared with binding of the constituent parts or a CD72Ig fusion protein, as follows. 2×10⁵ BJAB cells were added to wells of 96 well plates, centrifuged to pellet cells, and resuspended for binding. To the seeded plates, test proteins were added in a five-fold dilution series from 5 μg/ml down to 0.008 μg/ml. The cells with the proteins were incubated on ice for 45 minutes followed by centrifugation to pellet the cells. Resuspended pellets were washed twice with 200 ul of buffer to remove unbound proteins. One well containing no protein was treated similarly and served as a background control. To quantify binding, a goat anti-human antibody labeled with FITC (Fc-Specific) at 1:100 was added to each well, and the plates were again incubated on ice for 30 minutes. The plates were then washed once with 200 μl 1% FBS in PBS and the cells were re-suspended in 200 μl 1% FBS and analyzed by FACS using a FACSCalibur with CellQuest software (BD Biosciences).

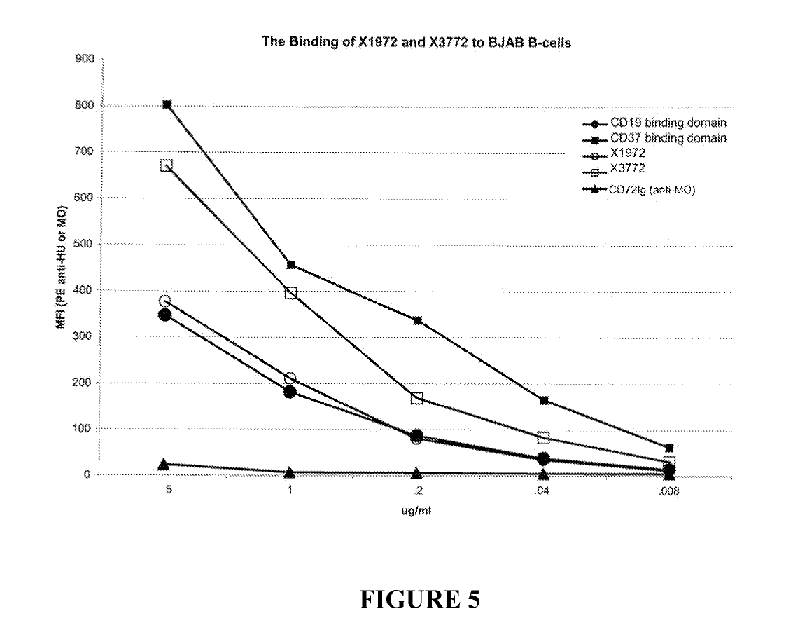

The data in FIG. 5 shows that binding of the Xceptors TRU-X1972 and TRU-X3772 to BJAB B-cells was comparable to binding of the constituent CD19 and CD37 binding domains.

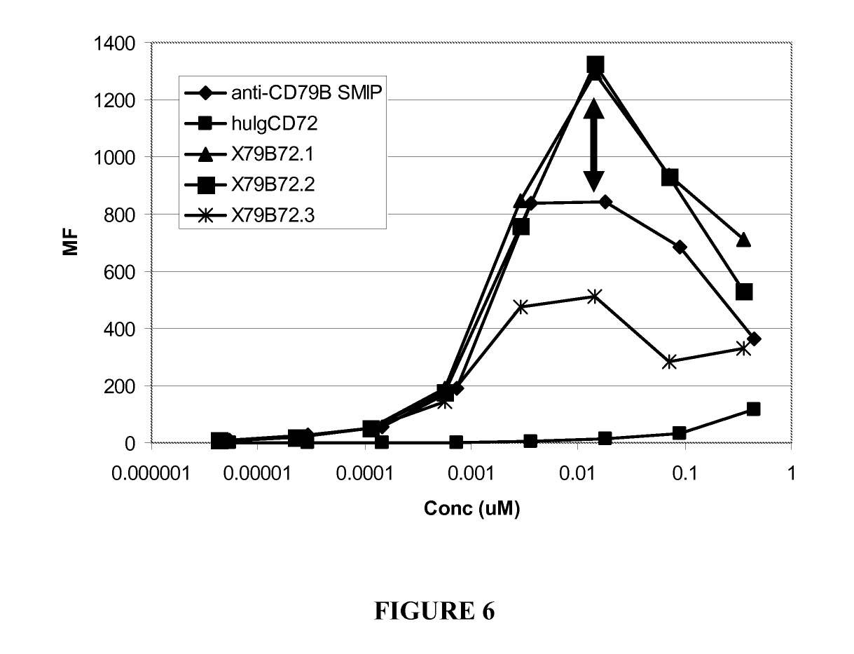

Binding of Xceptors X7972.1, X7972.2 and X7972.3 to Ramos cells was examined substantially as described above for BJAB cells. The results (FIG. 6) show that X7972.1 and X7972.3 can bind to Ramos cells better than CD79b SMIP, showing the avidity effect imparted by the CD79b ECD portion of the Xceptor molecules.

### Example 6

### Xceptor CDC Activity

Xceptors X3772 and X1972 were shown to have Complement-Dependent Cytotoxicity (CDC) activity. The experiment involved exposure of Ramos B-cells to CD19 and/or CD37 SMIPs (M0018 and CAS024, respectively) and Xceptors (X1972 and X3772, respectively) as well as an anti-CD20 SMIP (TRU-015), as described below and as shown in FIG. 2.

The experiment was initiated by adding from 5 to 2×10⁵ Ramos B-cells to wells of 96-well V-bottomed plates in 50 μl of Iscoves media with 10% FBS. The test compounds in Iscoves, (or Iscoves alone) were added to the wells in 50 μl at twice the indicated final concentration. The cells and reagents were incubated for 45 minutes at 37° C. The cells were washed 2.5 times by centrifugation and resuspension in Iscoves with no FBS and then resuspended in Iscoves with 10% human serum (Quidel, San Diego, Calif.) in 96-well plates at the indicated concentrations. The cells were incubated for 1 hour at 37° C. followed by a wash then resuspension in 125 μl cold PBS. Cells were transferred to FACs cluster tubes (CoStar, Corning, N.Y.) and 125 μl PBS with propidium iodide (Molecular Probes, Eugene, Oreg.) at 5 mg/ml was added. The cells were incubated with the propidium iodide for 15 minutes at room temperature in the dark and then placed on ice, quantitated, and analyzed on a FACsCalibur with CellQuest software (BD Biosciences).

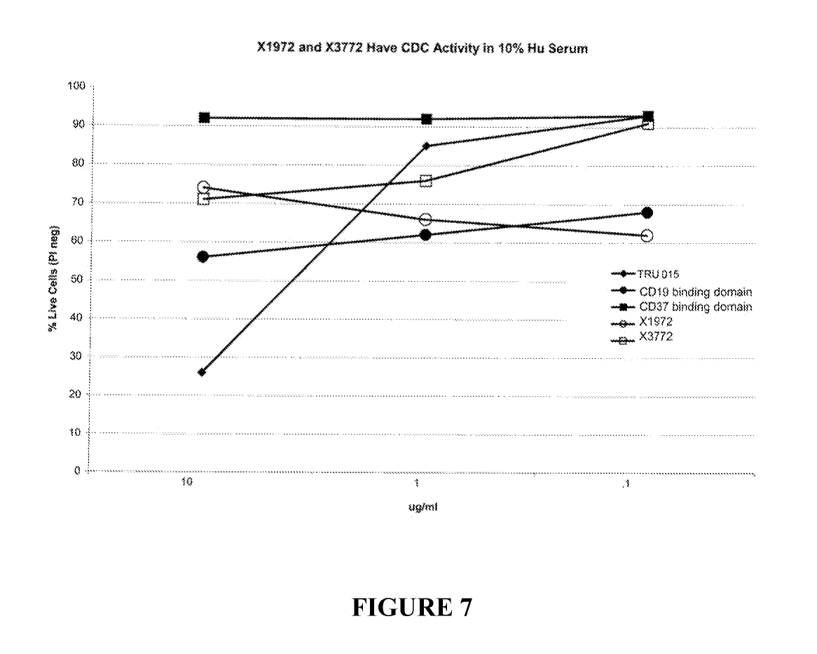

The results presented in FIG. 7 establish that the CD72-containing Xceptors exhibit CDC activity even when one of the targets, CD37, fails to support CDC activity when bound with the anti-CD37 SMIP.

### Example 7

### Xceptor Inhibition of REC-1, BJAB and DOHH2 B-Cell Growth

### (a) Inhibition of REC-1 B-Cell Growth

The ability of the Xceptors X3772 and X1972 to inhibit growth of the rituximab-resistant Mantle Cell Lymphoma Line Rec-1, as measured by reduction of thymidine uptake, was examined substantially as follows. Rec-1 (DSMZ ACC 584) cells were plated in 96-well plates at 1000-6000 cells/100 μl medium (RPMI-1640 10% FCS) per well. The X3772 protein and a comparator molecule, the anti-CD20 monoclonal protein rituximab, were added to the wells in a 10-fold dilution series that gave final protein concentrations ranging from 200 nM to 0.002 nM. As a control, some wells received media without added protein. Cells were incubated at 37° C. in a humidified incubator at 5% CO₂ for 96 hours. One microcurie of ³H-thymidine (Amersham) was added to each well and cells were incubated again at 37° C. in a humidified incubator at 5% CO₂ for an additional 4 hours. The cells were harvested onto UniFilter GF/C filter plates (Perkin Elmer) using a cell harvester (Packard). Microscint 20 (Packard) (25 μl/well) was added, and plates analyzed in TopCount NXT (Perkin Elmer/Packard). Each well was counted for one minute. The percent inhibition of cell proliferation was calculated by averaging all triplicates and normalizing to the media only control.

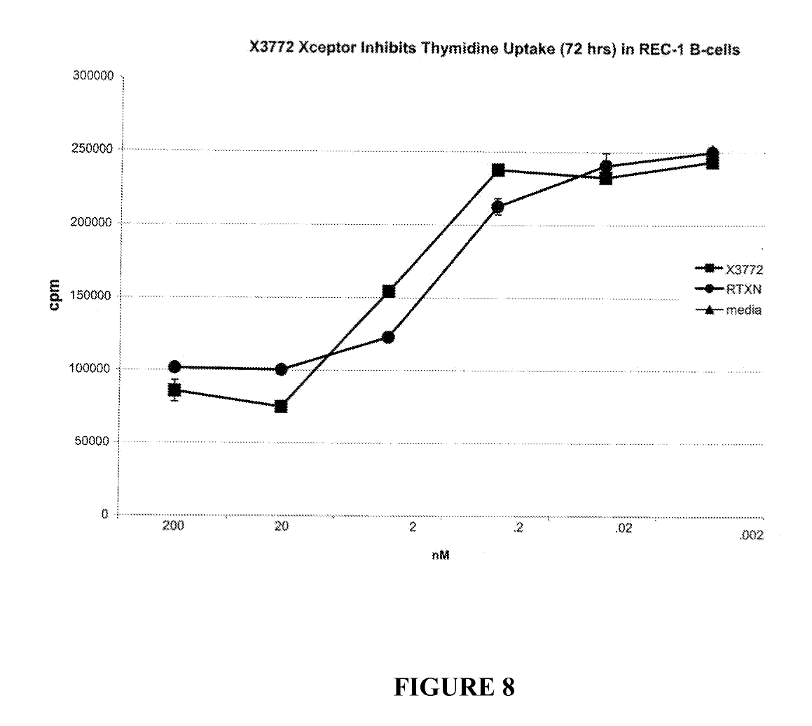

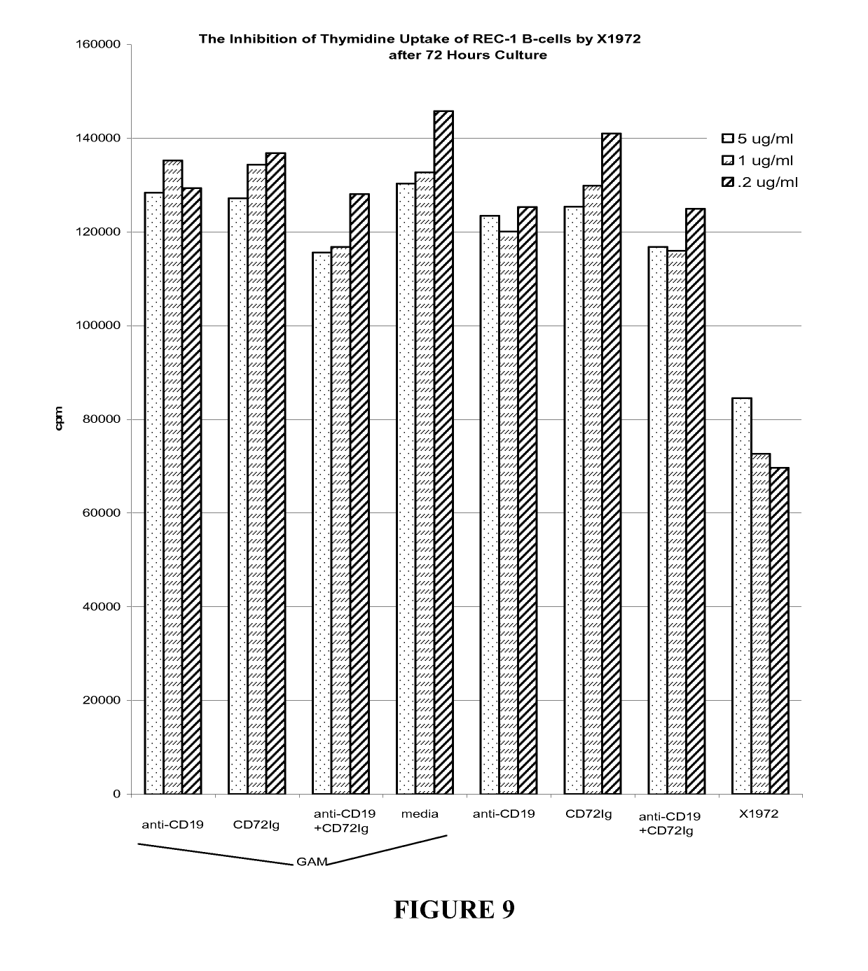

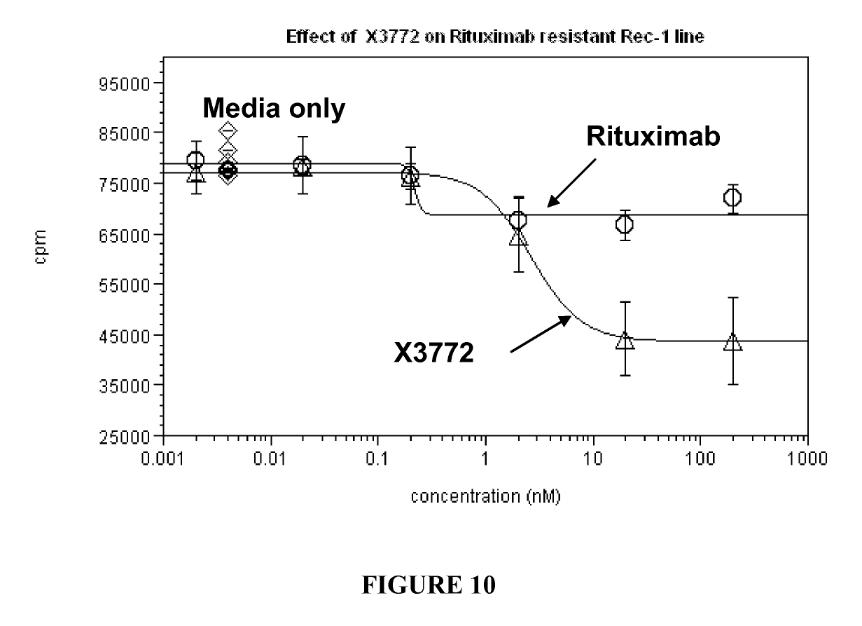

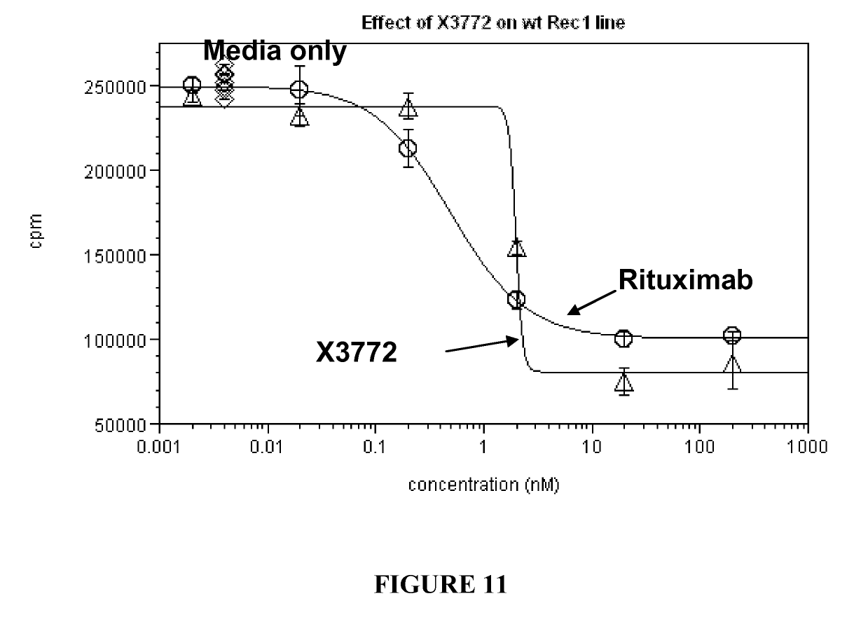

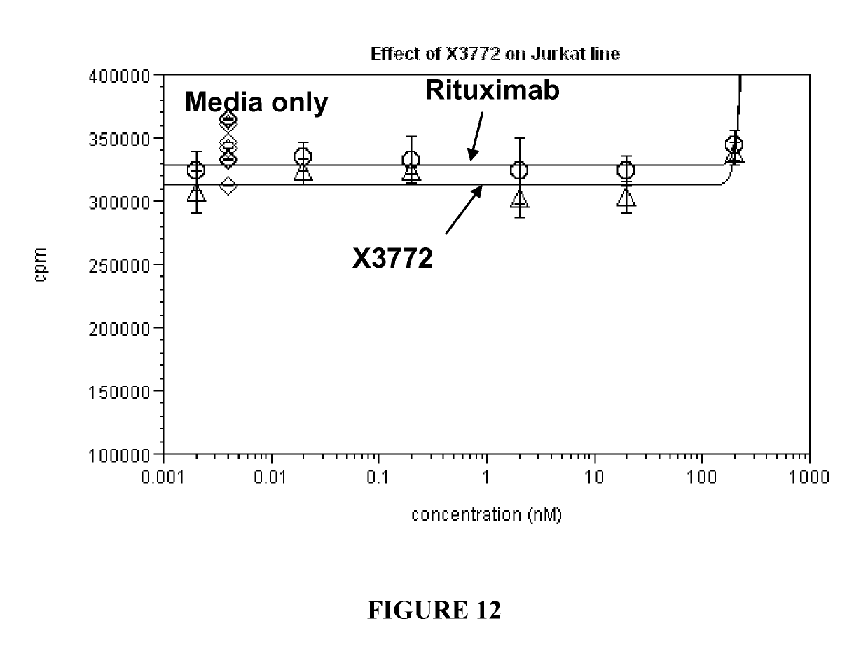

As shown in FIG. 8, the Xceptor X3772 exhibited strong growth inhibiting activity that was close to that of the anti-CD20 monoclonal. The results for the Xceptor X1972 are provided in FIG. 9 and demonstrate that the single agent alone (anti-CD19 SMIP and CD72Ig) did not have an effect on Rec-1 cells but the Xceptor X1972 produced a 50% growth inhibition. FIG. 10 shows that the growth of a rituximab-resistant Rec-1 cell line was not inhibited by rituximab, whereas X3772 significantly inhibited growth. Growth of the wild-type (wt) Rec-1 line was significantly inhibited by both rituximab and X3772 (FIG. 11). The growth inhibition produced by X3772 was specific to B cells as the molecule had no effect on Jurkat cells (see FIG. 12).

### (b) Inhibition of BAJB B-Cell Growth

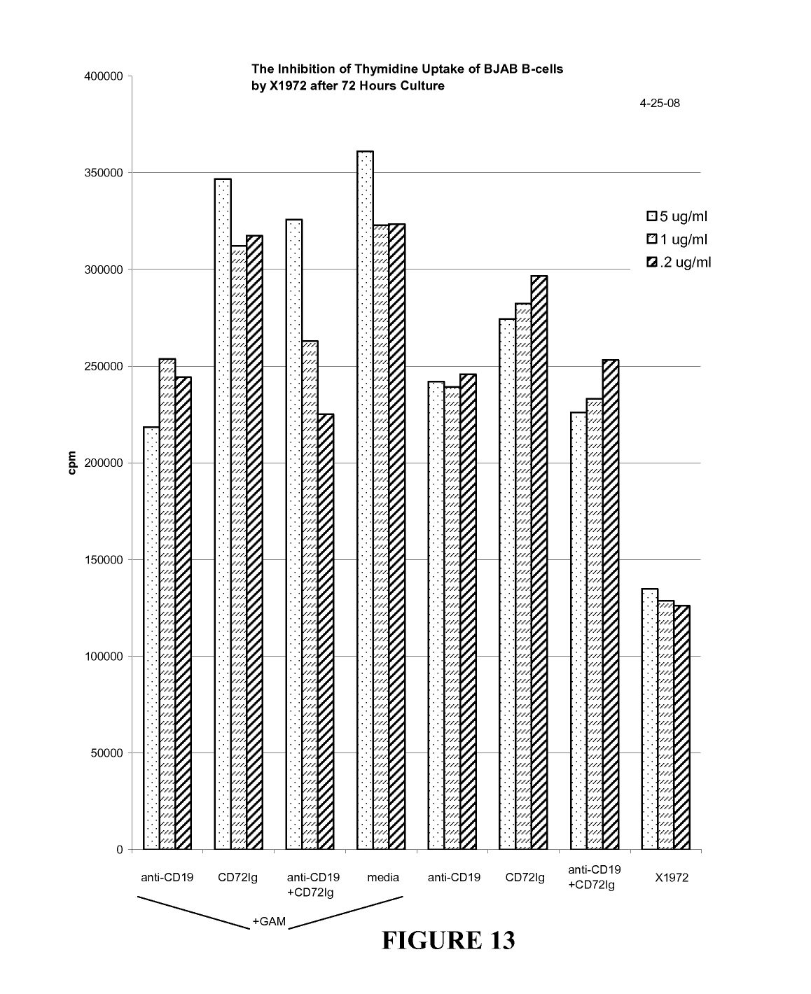

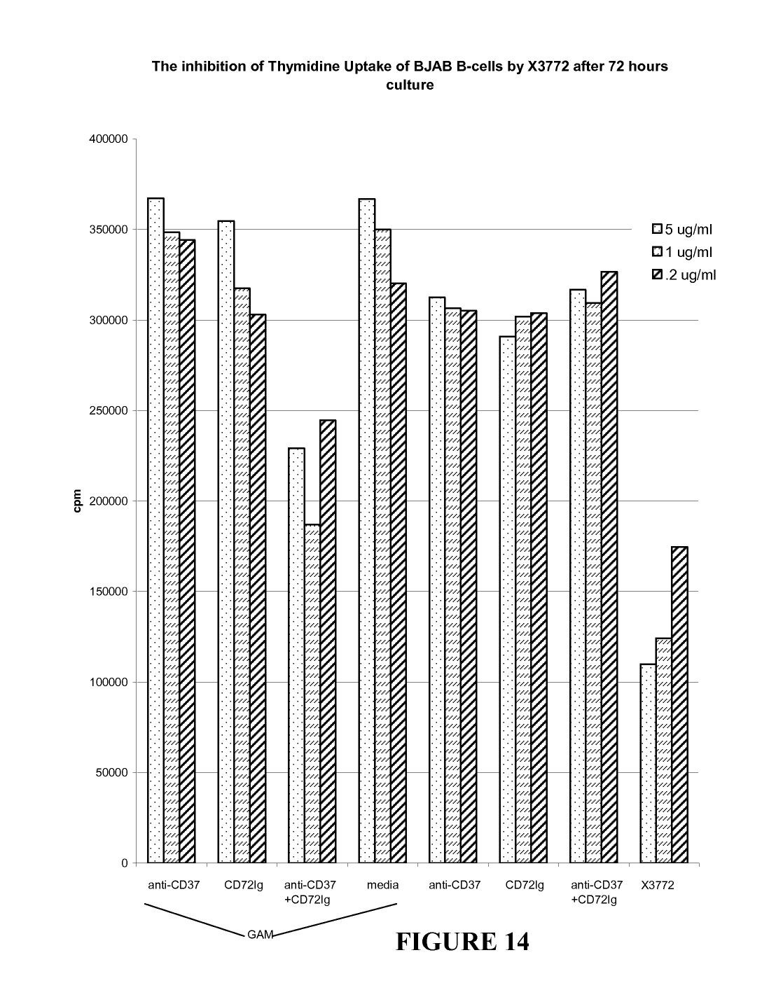

The ability of the Xceptors X1972 and X3772 to inhibit growth of the BJAB cell line, as measured by reduction of thymidine uptake, was examined substantially as described above for the REC-1 line. The results obtained for X1972 are shown in FIG. 13, with the results for X3772 being shown in FIG. 14. As shown in these Figures, single agent alone had no effect on the cell line, but the Xceptors X1972 and X3772 each produced a 50% growth inhibition.

### (c) Inhibition of DOHH2 Cell Growth

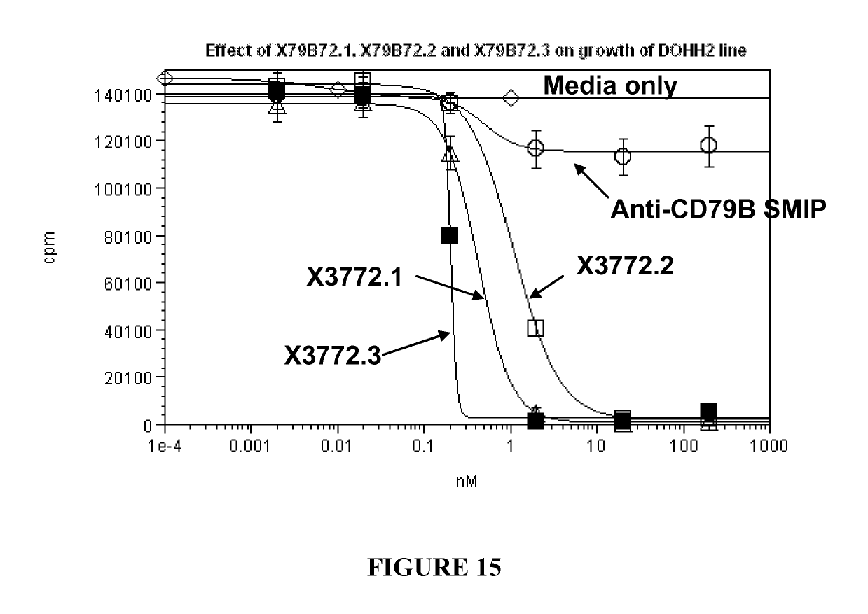

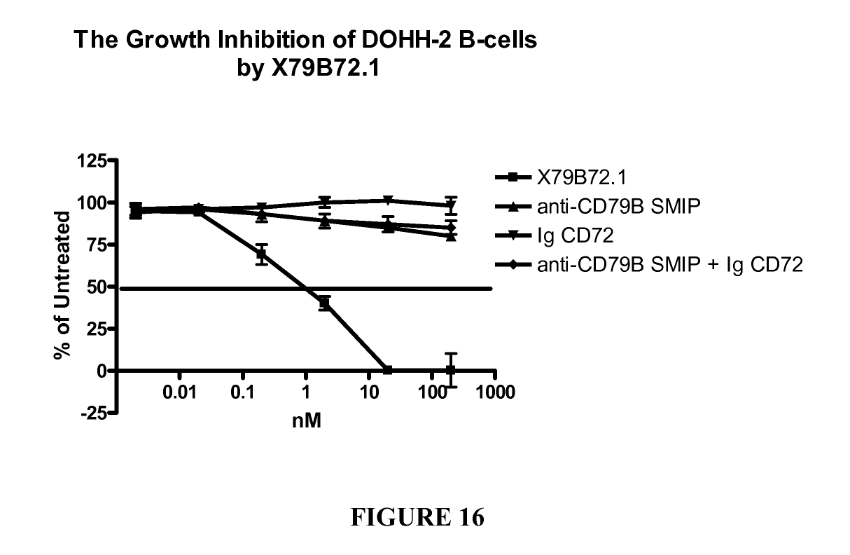

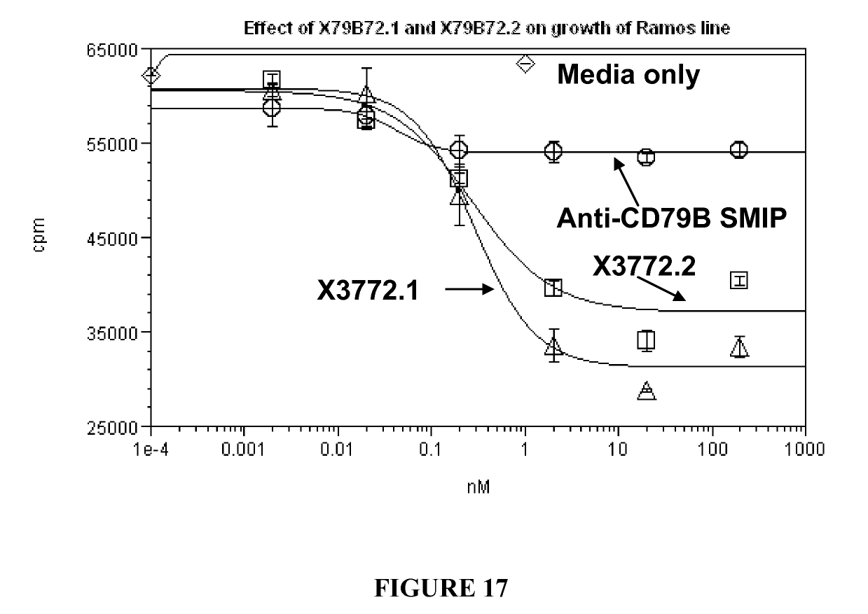

The ability of the Xceptors X7972.1, X7972.2 and X7972.3 to inhibit growth of the DOHH2 cell line, as measured by thymidine uptake, was also examined. As shown in FIG. 15, the Xceptors effectively blocked growth of DOHH2 cells whereas single agent alone (anti-CD79B SMIP) produced little effect. FIG. 16 demonstrates that neither single agent alone (CD72 Ig or CD72Ig) nor a combination of the two single agents inhibited growth of DOHH2 whereas the Xceptor molecule X7972.1 blocked growth of the cell line completely at concentrations greater than 20 ug/ml. The Xceptors X7972.1 and X7972.2 were also found to inhibit growth of other cells lines tested such as Ramos cells (FIG. 17) whereas rituximab had no effect.

### Example 8

### Xceptor Inhibition of Rituximab-Resistant DOHH2 B-Cell Growth

The anti-proliferative activity of Xceptor fusion proteins was examined in rituximab-resistant follicular lymphoma line DOHH-2RR as follows. DOHH-2RR was developed from the follicular lymphoma cell line DOHH-2 (DSMZ ACC 47) by repeated passage and growth over 3 months in the presence of 20 ng/ml rituximab with several washouts of the antibody to allow cell recovery. A ³H-thymidine cell proliferation assay was performed to determine the relative sensitivity of DOHH-2RR to the Xceptor X3772, variants X3772.1, X3772.2 and X3772.3 (SEQ ID NO: 13, 15 and 17, respectively), and rituximab.

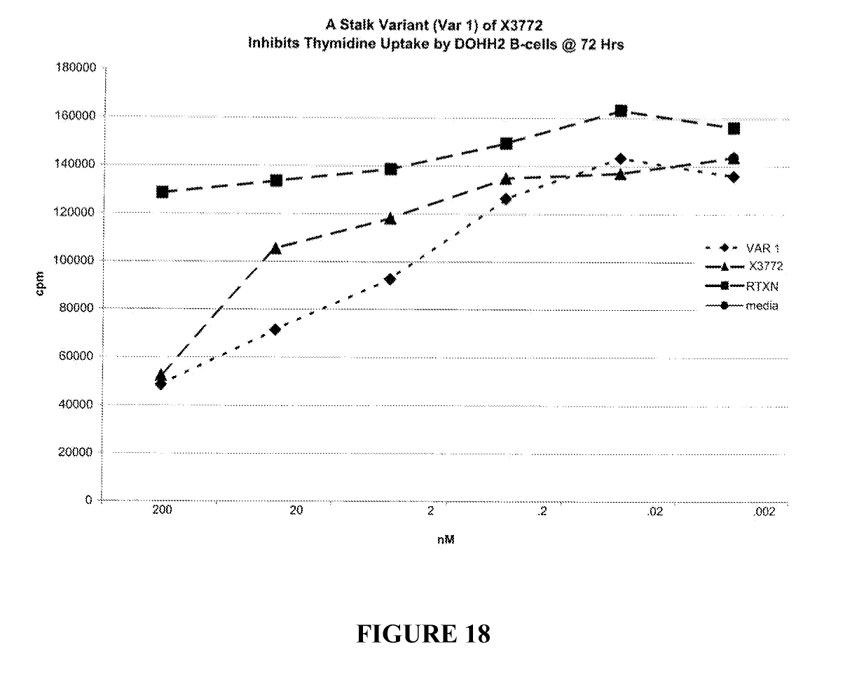

DOHH-2RR cells were plated in 96-well plates at 1000-6000 cells/100 μl medium (RPMI-1640 10% FCS) per well. The X3772 protein and rituximab were added to the wells in a 10-fold dilution series that gave final protein concentrations ranging from 200 nM to 0.002 nM. As a control, some wells received media without added protein. Cells were incubated at 37° C. in a humidified incubator at 5% CO₂ for 96 hours. One microcurie of ³H-thymidine (Amersham) was added to each well and cells were incubated again at 37° C. in a humidified incubator at 5% CO₂ for an additional 4 hours. The cells were harvested onto UniFilter GF/C filter plates (Perkin Elmer) using a cell harvester (Packard), Microscint 20 (Packard) (25 μl/well) was added, and plates analyzed in TopCount NXT (Perkin Elmer/Packard). Each well was counted for one minute. The percent inhibition of cell proliferation was calculated by averaging all triplicates and normalizing to the media only control. The Xceptors exhibited much stronger growth inhibition than rituximab with the variant with a shorter linker between the Fc region and the CD72 ectodomain (X3772.1) being more potent. The results for X3772.1 and X3772 are shown in FIG. 18.

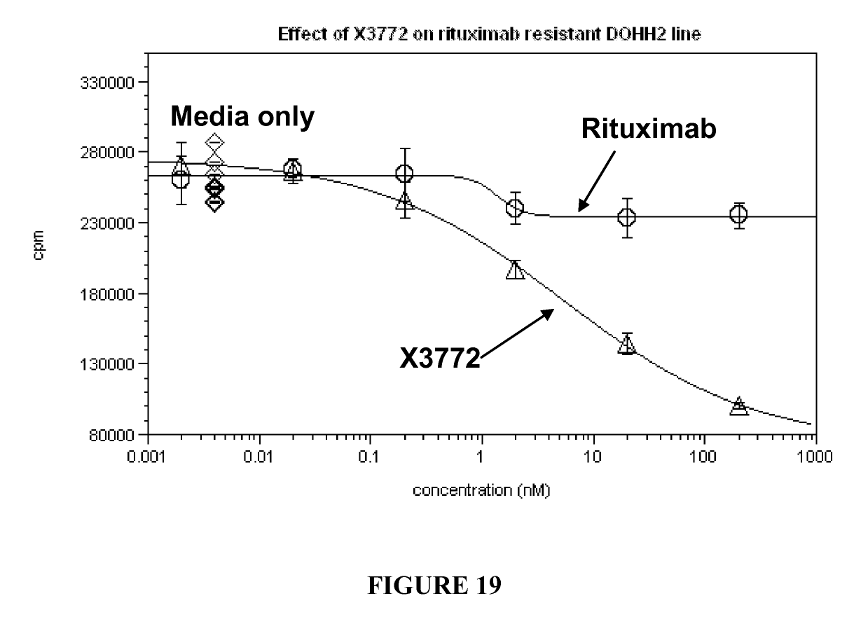

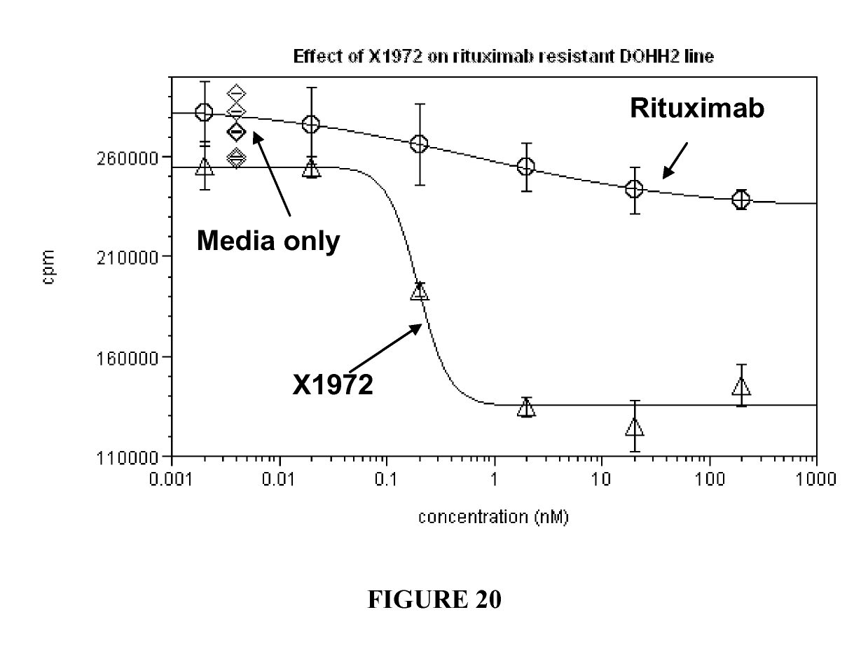

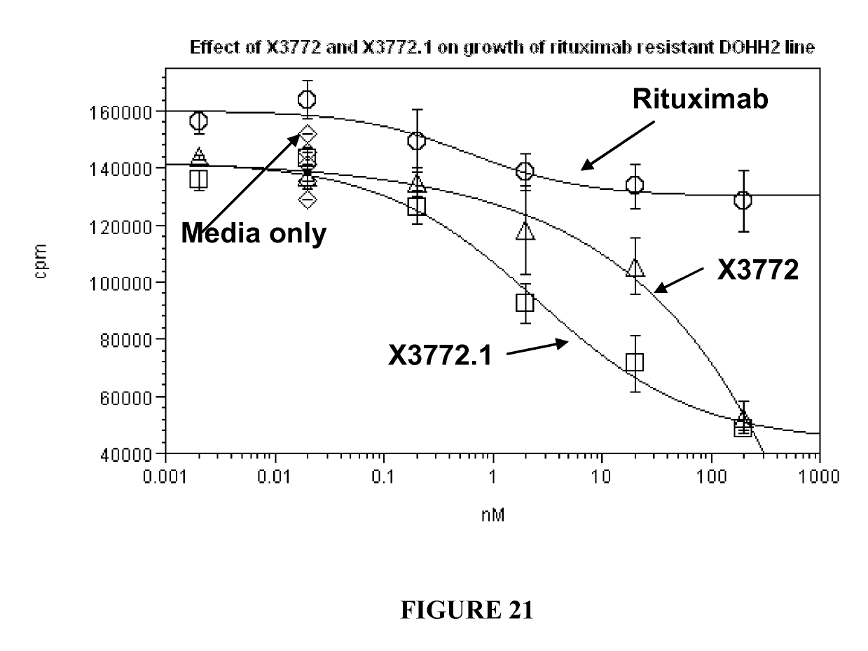

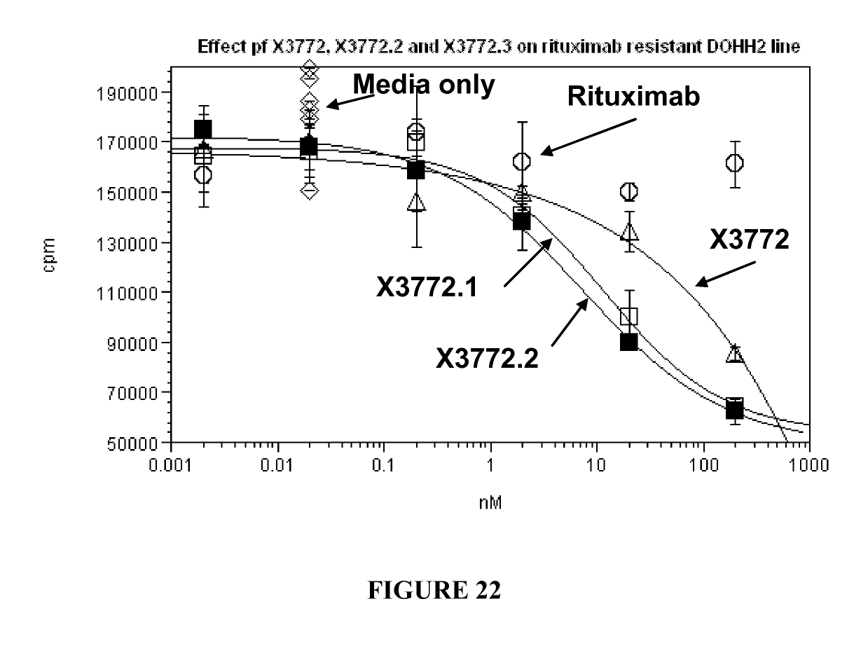

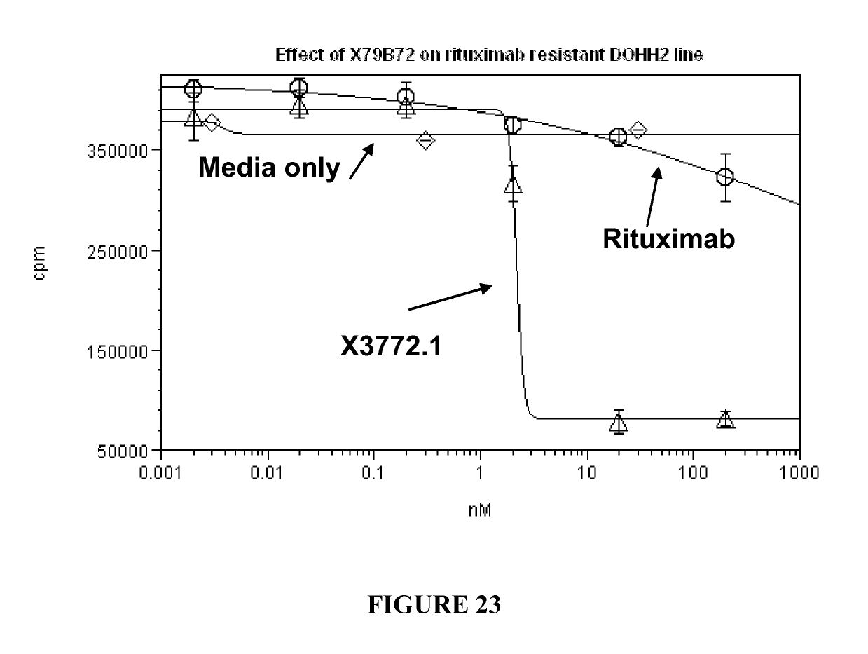

FIGS. 19 and 20 show that X3772 and X1972, respectively, but not rituximab, induced growth inhibition of DOHH-2RR cells. Variants of X3772 (X3772.1, X3772. 2 and X3772.3) were found to be more potent in inducing growth inhibition than X3772 (see FIGS. 21 and 22). The Xceptor X7992 was also found to inhibit growth of the DOHH2-RR cell line (FIG. 23), whereas rituximab had no effect.

### Example 9

### ADCC Activity of X3772 Having Linker Variants

Ramos cells (Burkitt's lymphoma line; ATCC CRL 1596) were labeled with 1.2 mCi/ml ⁵¹Cr sodium chromate (250 μCi/μg) for 2 hours at 37° C. in IMDM/10% FBS. The labeled cells were washed three times in RPMI/10% FBS and resuspended at 4×10⁵ cells/ml in RPMI. Heparinized, human whole blood was obtained from anonymous in-house donors and PBMC isolated by fractionation over Lymphocyte Separation Media (LSM, ICN Biomedical) gradients. Buffy coats were harvested and washed twice in RPMI/10% FBS prior to resuspension in RPMI/10% FBS at a final concentration of 5×10⁶ cells/ml. Cells were counted by trypan blue exclusion using a hemacytometer prior to use in subsequent assays. Reagent samples were added to RPMI medium with 10% FBS at 4 times the final concentration and three 25 fold serial dilutions for each reagent were prepared. These reagents were then added to 96-well U-bottom plates at 50 μl/well for the indicated final concentrations. The ⁵¹Cr-labeled BJAB cells were added to the plates at 50 μl/well (2×10⁴ cells/well). The PBMCs were then added to the plates at 100 μl/well (5×10⁵ cells/well) for a final ratio of 25:1 effector (PBMC):target (BJAB). Effectors and targets were added to medium alone to measure background killing. The ⁵¹Cr-labeled cells were added to medium alone to measure spontaneous release of ⁵¹Cr and to medium with 5% NP40 (cat. no. 28324, Pierce, Rockford, Ill.) to measure maximal release of ⁵¹Cr. Reactions were set up in triplicate wells of a 96-well plate.

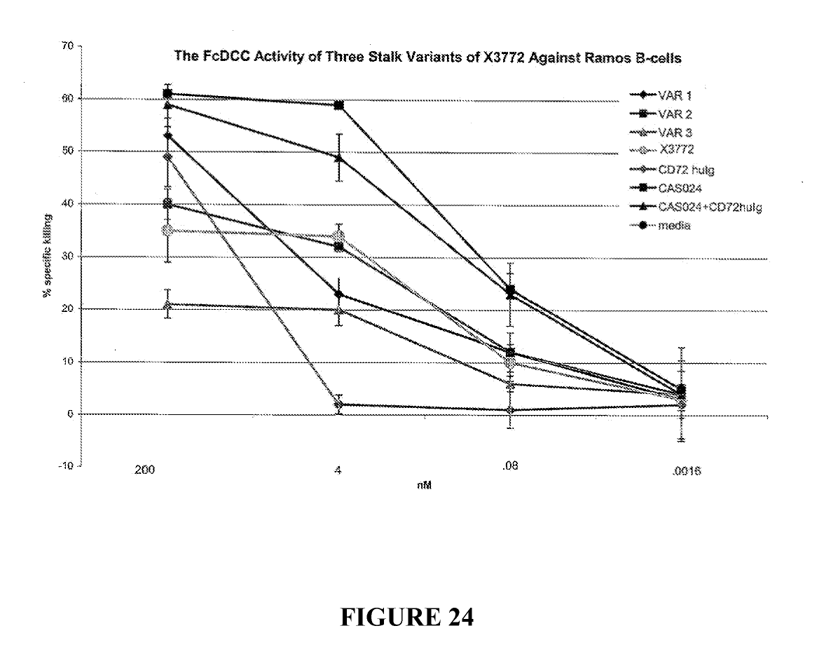

The Xceptor X3772, the Xceptors with linker variants (X3772.1, X3772.2 and X3772.3) and the SMIP and PIMS proteins (CAS024 and CD72huIg, respectively) were added to wells at a final concentration ranging from 0.016 nM to 200 nM as indicated in FIG. 24. For the combination of the SMIP plus PIMS the concentration stated is that for each of the added proteins. Reactions were allowed to proceed for 6 hours at 37° C. in 5% CO₂ prior to harvesting and counting. Twenty-five μl of the supernatant from each well were then transferred to a Luma Plate 96 (Perkin Elmer, Boston, Mass) and dried overnight at room temperature. CPM released was measured on a Packard TopCounNXT. Percent specific killing was calculated by subtracting (cpm {mean of triplicate samples} of sample−cpm spontaneous release)/(cpm maximal release-cpm spontaneous release) x100. Data are plotted as % specific killing versus protein concentration. The data demonstrate that the variant Xceptor molecules with the shorter and more flexible linkers mediate greater ADCC activity against the Ramos cells expressing the target antigen(s) although the activity over the dose range is lower than that of the anti-CD37 SMIP.

### Example 10

### Xceptor ADCC Activity on DOHH-2 B-Cells

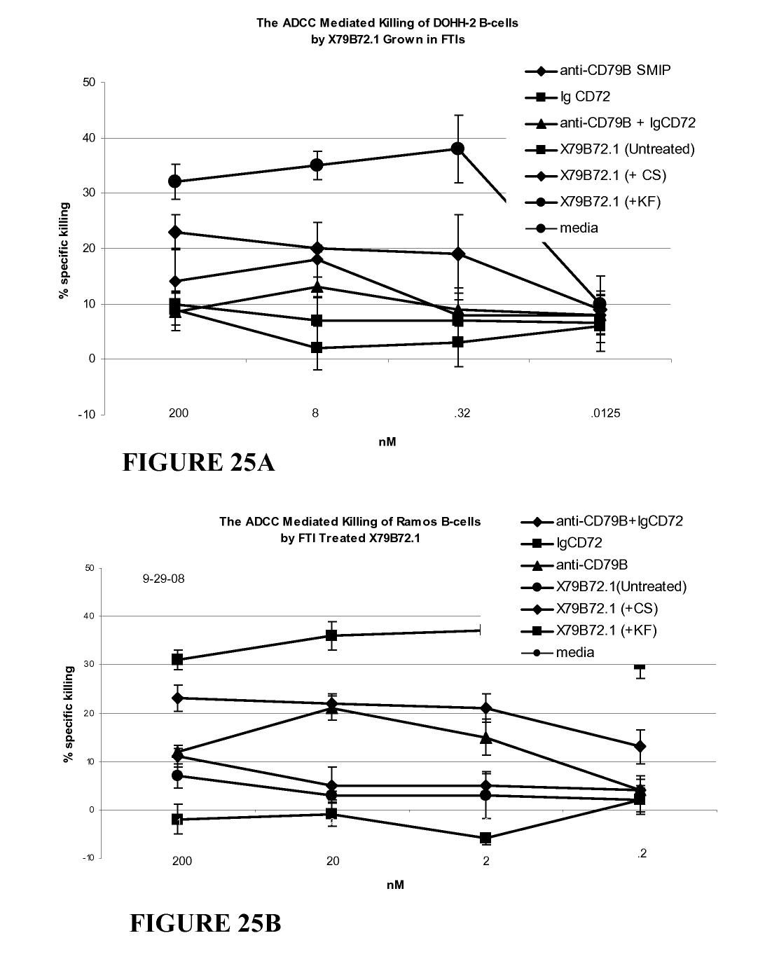

Xceptor X7972.1 ADCC activity against DOHH-2 cells was examined essentially as described above for Ramos cells. As shown in FIGS. 25A and B, the ADCC activity of X7992.1 was enhanced when transient expressed in HEK293 cells treated with the glucosidase inhibitor castanospermine (CS) or kifunensine (KF).

### Example 11

### Effect of Xceptor on DOHH-2 Cell Cycle

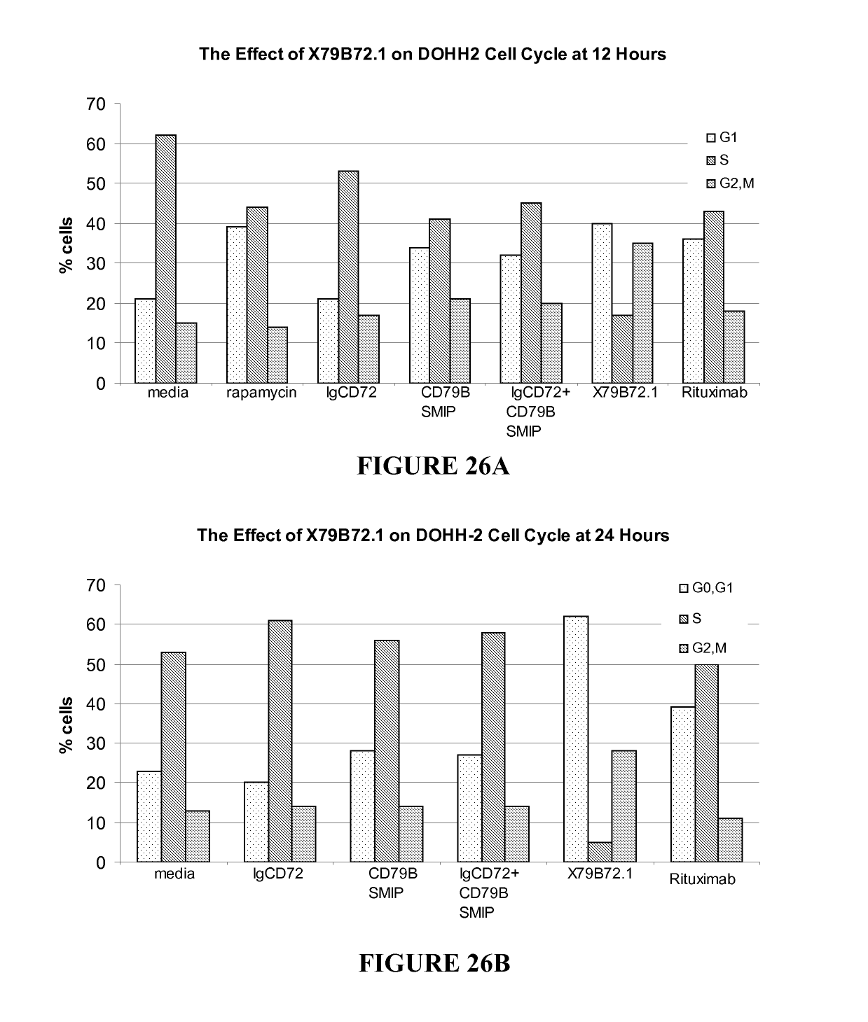

The cell-cycle effects were assessed by exposing lymphoma cells (DOHH2) to X7972.1, IgCD72, CD79B SMIP and Rituximab. More particularly, DOHH2 lymphoma cells (0.6×10⁵) were treated for 12 and 24 hours with 20 nM Rituximab, 20 nM X7972.1, 20 nM CD79B SMIP, 20 nM IgCD72 and 20 nM IgCD72+20 nM CD79B SMIP combination. Cultures were labeled for 45 minutes at 37° C. with 10 μM BrdU (bromodeoxyuridine). Following fixation, cells were stained with anti-BrdU-FITC antibody and counterstained with 7-AAD (7-Amino-Actinomycin D). Values obtained at 12 hours and 24 hours are shown in FIGS. 26A and B, respectively, and are the mean+/−SD of 4 replicate cultures. All sample data were analyzed at the same time and pooled for presentation using both the BrdU and 7-AAD incorporation dot plots.

The X7972.2 molecule arrested growth at the S phase, whereas the single agent alone or the combination of the two single agents had no effect. By comparison, rituximab produced very little arrest at the S phase.

While this invention has been described in conjunction with the specific embodiments outlined above, it is evident that many alternatives, modifications and variations will be apparent to those skilled in the art. Accordingly, the embodiments of this disclosure as set forth above are intended to be illustrative, not limiting. Various changes may be made without departing from the spirit and scope of this disclosure as defined in the following claims. All publications referenced herein are incorporated herein by reference as though fully set forth.

1. A multi-specific fusion protein, comprising a CD72-ligand binding domain linked to a B-cell protein binding domain by an intervening domain, wherein the B-cell protein is FCRL1, FCRL2, FCRL3, FCRL4, FCRL5, FCRL6, CD19, CD20, CD22, CD32b, CD37, CD79a, CD79b, CD267 or CD269.

2. The multi-specific fusion protein of claim 1 wherein the CD72-ligand binding domain is a CD72 ectodomain or sub-domain.

3. The multi-specific fusion protein of claim 1 wherein the CD72-ligand binding domain comprises an amino acid sequence as set forth in SEQ ID NO: 1.

4. The multi-specific fusion protein of claim 1, wherein the CD72-ligand binding domain comprises amino acids 221-359 or 233-359 of SEQ ID NO:1.

5. (canceled)

6. The multi-specific fusion protein of claim 1 wherein the B-cell protein binding domain is specific for CD19 or CD37.

7. (canceled)

8. The multi-specific fusion protein of claim 1 wherein the B-cell protein binding domain is a Fab, scFv, a domain antibody, or a heavy chain-only antibody.

9. (canceled)

10. The multi-specific fusion protein of claim 6 wherein the B-cell protein binding domain specific for CD19 or CD37 comprises a light chain variable region containing CDR1, CDR2, and CDR3 sequences that are each at least 80% identical to at least one light chain variable region CDR1, CDR2, and CDR3, respectively, as set forth in SEQ ID NO:9 or 11, respectively.

11. The multi-specific fusion protein of claim 6 wherein the B-cell protein binding domain specific for CD19 or CD37 comprises a heavy chain variable region containing CDR1, CDR2, and CDR3 sequences that are each at least 80% identical to at least one heavy chain variable region CDR1, CDR2, and CDR3, respectively, as set forth in SEQ ID NO:9 or 11, respectively.

12. The multi-specific fusion protein of claim 6 wherein the B-cell protein binding domain specific for CD19 or CD37 comprises a light chain variable region containing CDR1, CDR2, and CDR3 sequences that are each at least 80% identical to at least one light chain variable region CDR1, CDR2, and CDR3, respectively, as set forth in of SEQ ID NO:9 or 11, respectively, and comprises a heavy chain variable region containing CDR1, CDR2, and CDR3 sequences that are each at least 80% identical to at least one heavy chain variable region. CDR1, CDR2, and CDR3, respectively, as set forth in SEQ ID NO:9 or 11, respectively.

13. The multi-specific fusion protein of claim 1 wherein the intervening domain comprises an immunoglobulin constant region or sub-region disposed between the CD72-ligand binding domain and the binding domain specific for a B-cell protein.

14. The multi-specific fusion protein of claim 1 wherein the intervening domain comprises an immunoglobulin constant region disposed between a first and a second linker.

15. The multi-specific fusion protein of claim 14 wherein the first and second linkers are independently selected from SEQ ID NO: 18-147.

16. The multi-specific fusion protein of claim 14 wherein the intervening domain comprises a human immunoglobulin Fc region, albumin, transferrin, or a scaffold domain that binds a serum protein.

17. The multi-specific fusion protein of claim 1 wherein the intervening domain comprises a structure, from amino-terminus to carboxy-terminus, as follows:
-L1-X-L2-
wherein:
L1 and L2 are each independently a linker comprising from two to about 150 amino acids; and
X is an immunoglobulin constant region or sub-region, albumin, transferrin, or another serum protein binding protein.

18. The multi-specific fusion protein of claim 17 wherein L1 is a human immunoglobulin hinge region, optionally mutated to replace one or more cysteines with other amino acids.

19. (canceled)

20. The multi-specific fusion protein of claim 1 wherein the intervening domain is a dimerization domain.

21. The multi-specific fusion protein of claim 1 having the following structure:
N-BD1-X-L2-ED2-C
wherein:
BD1 is a CD19 or CD37 binding domain that is at least about 90% identical to a binding domain found in SEQ ID NO:9 or 11, respectively;
—X— is -L1-CH2CH3-, wherein L1 is the first IgG1 hinge, optionally mutated by substituting the first cysteine and wherein —CH2CH3- is the CH2CH3 region of an IgG1 Fc domain;
L2 is a linker selected from SEQ ID NO: 1-147; and
BD2 is a CD72-ligand binding domain specific for CD72 ligand CD100 or CD5.

22. A composition comprising the multi-specific fusion protein according to claim 1 and a pharmaceutically acceptable carrier, diluent, or excipient.

23. The composition of claim 22 wherein the multi-specific fusion protein exists as a dimer or a multimer in the composition.

24. A polynucleotide encoding the multi-specific fusion protein according to claim 1.

25. An expression vector comprising the polynucleotide according to claim 24, which is operably linked to an expression control sequence.

26. A host cell comprising the expression vector according to claim 25.

27. A method for treating a subject with a B-cell related inflammatory or malignant condition comprising the administration of a therapeutically effective amount of the multi-specific fusion protein of claim 1.

28. The method of claim 27 wherein the B-cell related inflammatory condition is rheumatoid arthritis, pemphigus, systemic lupus erythematosus, idiopathic thrombocytopenic purpura, or autoimmune hemolytic anemia.

29. A method of modulating a B cell activity or proliferation in a subject comprising administering an effective amount of the multi-specific fusion protein of claim 1 to a subject in need thereof.

30. A method of producing a multi-specific fusion protein comprising culturing the host cell of claim 26 in a medium and expressing the protein.


SEQUENCE LISTING (184 sequences)

SEQ ID NO: 1 - protein, 359 aa, Human
MAEAITYADLRFVKAPLKKSISSRLGQDPGADDDGEITYENVQVPAVLGVPSSLASSVLGDKAAVKSEQPTASWRAVTSPAVGRILPCRTTCLRYLLLGLLLTCLLLGVTAICLGVRYLQVSQQLQQTNRVLEVTNSSLRQQLRLKITQLGQSAEDLQGSRRELAQSQEALQVEQRAHQAAEGQLQACQADRQKTKETLQSEEQQRRALEQKLSNMENRLKPFFTCGSADTCCPSGWIMHQKSCFYISLTSKNWQESQKQCETLSSKLATFSEIYPQSHSYYFLNSLLPNGGSGNSYWTGLSSNKDWKLTDDTQRTRTYAQSSKCNKVHKTWSWWTLESESCRSSLPYICEMTAFRFPD

SEQ ID NO: 2 - protein, 243 aa, Human
RYLQVSQQLQQTNRVLEVTNSSLRQQLRLKITQLGQSAEDLQGSRRELAQSQEALQVEQRAHQAAEGQLQACQADRQKTKETLQSEEQQRRALEQKLSNMENRLKPFFTCGSADTCCPSGWIMHQKSCFYISLTSKNWQESQKQCETLSSKLATFSEIYPQSHSYYFLNSLLPNGGSGNSYWTGLSSNKDWKLTDDTQRTRTYAQSSKCNKVHKTWSWWTLESESCRSSLPYICEMTAFRFPD

SEQ ID NO: 3 - DNA, 732 bp, Human
cgctatctgcaggtgtctcagcagctccagcagacgaacagggttctggaagtcactaacagcagcctgaggcagcagctccgcctcaagataacgcagctgggacagagtgcagaggatctgcaggggtccaggagagagctggcgcagagtcaggaagcactacaggtggaacagagggctcatcaggcggccgaagggcagctacaggcctgccaggcagacagacagaagacgaaggagaccttgcaaagtgaggagcaacagaggagggccttggagcagaagctgagcaacatggagaacagactgaagcccttcttcacatgcggctcagcagacacctgctgtccgtcgggatggataatgcatcagaaaagctgcttttacatctcacttacttcaaaaaattggcaggagagccaaaaacaatgtgaaactctgtcttccaagctggccacattcagtgaaatttatccacaatcacactcttactacttcttaaattcactgttgccaaatggtggttcagggaattcatattggactggcctcagctctaacaaggattggaagttgactgatgatacacaacgcactaggacttatgctcaaagctcaaaatgtaacaaggtacataaaacttggtcatggtggacactggagtcagagtcatgtagaagttctcttccctacatctgtgagatgacagctttcaggtttccagattag

SEQ ID NO: 4 - DNA, 1503 bp, Artificial Sequence
  Made in a lab
atggaagcaccagcgcagcttctcttcctcctgctactctggctcccagataccaccggttcctcgagccagcccagagggcccacaatcaagccctgtcctccatgcaaatgcccggctccaaatcttcttggtggttcatccgtcttcatcttccctccaaagatcaaggatgtactcatgatctccctgagccccatagtcacatgtgtggtggtggatgtgagcgaggacgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggactaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaacgtacgcgctatctgcaggtgtctcagcagctccagcagacgaacagggttctggaagtcactaacagcagcctgaggcagcagctccgcctcaagataacgcagctgggacagagtgcagaggatctgcaggggtccaggagagagctggcgcagagtcaggaagcactacaggtggaacagagggctcatcaggcggccgaagggcagctacaggcctgccaggcagacagacagaagacgaaggagaccttgcaaagtgaggagcaacagaggagggccttggagcagaagctgagcaacatggagaacagactgaagcccttcttcacatgcggctcagcagacacctgctgtccgtcgggatggataatgcatcagaaaagctgcttttacatctcacttacttcaaaaaattggcaggagagccaaaaacaatgtgaaactctgtcttccaagctggccacattcagtgaaatttatccacaatcacactcttactacttcttaaattcactgttgccaaatggtggttcagggaattcatattggactggcctcagctctaacaaggattggaagttgactgatgatacacaacgcactaggacttatgctcaaagctcaaaatgtaacaaggtacataaaacttggtcatggtggacactggagtcagagtcatgtagaagttctcttccctacatctgtgagatgacagctttcaggtttccagat

SEQ ID NO: 5 - protein, 501 aa, Artificial Sequence
  Made in a lab
MEAPAQLLFLLLLWLPDTTGSSSQPRGPTIKPCPPCKCPAPNLLGGSSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKRTRYLQVSQQLQQTNRVLEVTNSSLRQQLRLKITQLGQSAEDLQGSRRELAQSQEALQVEQRAHQAAEGQLQACQADRQKTKETLQSEEQQRRALEQKLSNMENRLKPFFTCGSADTCCPSGWIMHQKSCFYISLTSKNWQESQKQCETLSSKLATFSEIYPQSHSYYFLNSLLPNGGSGNSYWTGLSSNKDWKLTDDTQRTRTYAQSSKCNKVHKTWSWWTLESESCRSSLPYICEMTAFRFPD

SEQ ID NO: 6 - DNA, 1500 bp, Artificial Sequence
  Made in a lab
atggaagcaccagcgcagcttctcttcctcctgctactctggctcccagataccaccggttcctcgagcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaagcgtacgcgctatctgcaggtgtctcagcagctccagcagacgaacagggttctggaagtcactaacagcagcctgaggcagcagctccgcctcaagataacgcagctgggacagagtgcagaggatctgcaggggtccaggagagagctggcgcagagtcaggaagcactacaggtggaacagagggctcatcaggcggccgaagggcagctacaggcctgccaggcagacagacagaagacgaaggagaccttgcaaagtgaggagcaacagaggagggccttggagcagaagctgagcaacatggagaacagactgaagcccttcttcacatgcggctcagcagacacctgctgtccgtcgggatggataatgcatcagaaaagctgcttttacatctcacttacttcaaaaaattggcaggagagccaaaaacaatgtgaaactctgtcttccaagctggccacattcagtgaaatttatccacaatcacactcttactacttcttaaattcactgttgccaaatggtggttcagggaattcatattggactggcctcagctctaacaaggattggaagttgactgatgatacacaacgcactaggacttatgctcaaagctcaaaatgtaacaaggtacataaaacttggtcatggtggacactggagtcagagtcatgtagaagttctcttccctacatctgtgagatgacagctttcaggtttccagat

SEQ ID NO: 7 - protein, 500 aa, Artificial Sequence
  Made in a lab
MEAPAQLLFLLLLWLPDTTGSSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRTRYLQVSQQLQQTNRVLEVTNSSLRQQLRLKITQLGQSAEDLQGSRRELAQSQEALQVEQRAHQAAEGQLQACQADRQKTKETLQSEEQQRRALEQKLSNMENRLKPFFTCGSADTCCPSGWIMHQKSCFYISLTSKNWQESQKQCETLSSKLATFSEIYPQSHSYYFLNSLLPNGGSGNSYWTGLSSNKDWKLTDDTQRTRTYAQSSKCNKVHKTWSWWTLESESCRSSLPYICEMTAFRFPD

SEQ ID NO: 8 - DNA, 2250 bp, Artificial Sequence
  Made in a lab
atggaagcaccagcgcagcttctcttcctcctgctactctggctcccagataccaccggtgacattgtgctgacccaatctccagcttctttggctgtgtctctagggcagagggccaccatctcctgcaaggccagccaaagtgttgattatgatggtgatagttatttgaactggtaccaacagattccaggacagccacccaaactcctcatctatgatgcatccaatctagtttctgggatcccacccaggtttagtggcagtgggtctgggacagacttcaccctcaacatccatcctgtggagaaggtggatgctgcaacctatcactgccagcaaagtactgaggatccgtggacgttcggtggaggcaccaagctggaaatcaaaggtggcggtggttcgggcggtggtgggtcgggtggcggcggagctagccaggttcagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttggcctggagatggtgatactaactacaatggaaagttcaagggtaaagccactctgactgcagacgaatcctccagcacagcctacatgcaactcagcagcctagcatctgaggactctgcggtctatttctgtgcaagacgggagactacgacggtaggccgttattactatgctatggactactggggtcaaggaacctcagtcaccgtctcctcgagcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaagcgtacgcgctatctgcaggtgtctcagcagctccagcagacgaacagggttctggaagtcactaacagcagcctgaggcagcagctccgcctcaagataacgcagctgggacagagtgcagaggatctgcaggggtccaggagagagctggcgcagagtcaggaagcactacaggtggaacagagggctcatcaggcggccgaagggcagctacaggcctgccaggcagacagacagaagacgaaggagaccttgcaaagtgaggagcaacagaggagggccttggagcagaagctgagcaacatggagaacagactgaagcccttcttcacatgcggctcagcagacacctgctgtccgtcgggatggataatgcatcagaaaagctgcttttacatctcacttacttcaaaaaattggcaggagagccaaaaacaatgtgaaactctgtcttccaagctggccacattcagtgaaatttatccacaatcacactcttactacttcttaaattcactgttgccaaatggtggttcagggaattcatattggactggcctcagctctaacaaggattggaagttgactgatgatacacaacgcactaggacttatgctcaaagctcaaaatgtaacaaggtacataaaacttggtcatggtggacactggagtcagagtcatgtagaagttctcttccctacatctgtgagatgacagctttcaggtttccagattag

SEQ ID NO: 9 - protein, 749 aa, Artificial Sequence [sequence omitted]

SEQ ID NO: 10 - DNA, 2247 bp, Artificial Sequence
  Made in a lab
atggaagccccagctcagcttctcttcctcctgctactctggctcccagataccaccggagaggtgcagctggtgcagtctggagcagaggtgaaaaagcccggagagtctctgaagatttcctgtaagggctccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctttcgactcctggggccagggcaccctggtcactgtctcctctgggggtggaggctctggtggcggtggctctggcggaggtggatccggtggcggcggatctggcgggggtggctctgaaattgtgttgacacagtctccagccaccctgtctttgtctccaggcgaaagagccaccctctcctgccgagcaagtgaaaatgtttacagctacttagcctggtaccaacagaaacctggccaggctcctaggctcctcatctattttgcaaaaaccttagcagaaggaattccagccaggttcagtggcagtggctccgggacagacttcactctcaccatcagcagcctagagcctgaagattttgcagtttattactgtcaacatcattccgataatccgtggacattcggccaagggaccaaggtggaaatcaaaggtgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaagcgtacgcgctatctgcaggtgtctcagcagctccagcagacgaacagggttctggaagtcactaacagcagcctgaggcagcagctccgcctcaagataacgcagctgggacagagtgcagaggatctgcaggggtccaggagagagctggcgcagagtcaggaagcactacaggtggaacagagggctcatcaggcggccgaagggcagctacaggcctgccaggcagacagacagaagacgaaggagaccttgcaaagtgaggagcaacagaggagggccttggagcagaagctgagcaacatggagaacagactgaagcccttcttcacatgcggctcagcagacacctgctgtccgtcgggatggataatgcatcagaaaagctgcttttacatctcacttacttcaaaaaattggcaggagagccaaaaacaatgtgaaactctgtcttccaagctggccacattcagtgaaatttatccacaatcacactcttactacttcttaaattcactgttgccaaatggtggttcagggaattcatattggactggcctcagctctaacaaggattggaagttgactgatgatacacaacgcactaggacttatgctcaaagctcaaaatgtaacaaggtacataaaacttggtcatggtggacactggagtcagagtcatgtagaagttctcttccctacatctgtgagatgacagctttcaggtttccagattag

SEQ ID NO: 11 - protein, 748 aa, Artificial Sequence [sequence omitted]

SEQ ID NO: 12 - DNA, 1995 bp, Artificial Sequence
  Made in a lab
atggaagccccagctcagcttctcttcctcctgctactctggctcccagataccaccggagaggtgcagctggtgcagtctggagcagaggtgaaaaagcccggagagtctctgaagatttcctgtaagggctccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctttcgactcctggggccagggcaccctggtcactgtctcctctgggggtggaggctctggtggcggtggctctggcggaggtggatccggtggcggcggatctggcgggggtggctctgaaattgtgttgacacagtctccagccaccctgtctttgtctccaggcgaaagagccaccctctcctgccgagcaagtgaaaatgtttacagctacttagcctggtaccaacagaaacctggccaggctcctaggctcctcatctattttgcaaaaaccttagcagaaggaattccagccaggttcagtggcagtggctccgggacagacttcactctcaccatcagcagcctagagcctgaagattttgcagtttattactgtcaacatcattccgataatccgtggacattcggccaagggaccaaggtggaaatcaaaggtgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaagcgtacgcaaagtgaggagcaacagaggagggccttggagcagaagctgagcaacatggagaacagactgaagcccttcttcacatgcggctcagcagacacctgctgtccgtcgggatggataatgcatcagaaaagctgcttttacatctcacttacttcaaaaaattggcaggagagccaaaaacaatgtgaaactctgtcttccaagctggccacattcagtgaaatttatccacaatcacactcttactacttcttaaattcactgttgccaaatggtggttcagggaattcatattggactggcctcagctctaacaaggattggaagttgactgatgatacacaacgcactaggacttatgctcaaagctcaaaatgtaacaaggtacataaaacttggtcatggtggacactggagtcagagtcatgtagaagttctcttccctacatctgtgagatgacagctttcaggtttccagat

SEQ ID NO: 13 - protein, 665 aa, Artificial Sequence
  Made in a lab
MEAPAQLLFLLLLWLPDTTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPFDSWGQGTLVTVSSGGGGSGGGGSGGGGSGGGGSGGGGSEIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRTQSEEQQRRALEQKLSNMENRLKPFFTCGSADTCCPSGWIMHQKSCFYISLTSKNWQESQKQCETLSSKLATFSEIYPQSHSYYFLNSLLPNGGSGNSYWTGLSSNKDWKLTDDTQRTRTYAQSSKCNKVHKTWSWWTLESESCRSSLPYICEMTAFRFPD

SEQ ID NO: 14 - DNA, 1965 bp, Artificial Sequence
  Made in a lab
atggaagccccagctcagcttctcttcctcctgctactctggctcccagataccaccggagaggtgcagctggtgcagtctggagcagaggtgaaaaagcccggagagtctctgaagatttcctgtaagggctccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctttcgactcctggggccagggcaccctggtcactgtctcctctgggggtggaggctctggtggcggtggctctggcggaggtggatccggtggcggcggatctggcgggggtggctctgaaattgtgttgacacagtctccagccaccctgtctttgtctccaggcgaaagagccaccctctcctgccgagcaagtgaaaatgtttacagctacttagcctggtaccaacagaaacctggccaggctcctaggctcctcatctattttgcaaaaaccttagcagaaggaattccagccaggttcagtggcagtggctccgggacagacttcactctcaccatcagcagcctagagcctgaagattttgcagtttattactgtcaacatcattccgataatccgtggacattcggccaagggaccaaggtggaaatcaaaggtgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaagcgtacggagcagaagctgagcaacatggagaacagactgaagcccttcttcacatgcggctcagcagacacctgctgtccgtcgggatggataatgcatcagaaaagctgcttttacatctcacttacttcaaaaaattggcaggagagccaaaaacaatgtgaaactctgtcttccaagctggccacattcagtgaaatttatccacaatcacactcttactacttcttaaattcactgttgccaaatggtggttcagggaattcatattggactggcctcagctctaacaaggattggaagttgactgatgatacacaacgcactaggacttatgctcaaagctcaaaatgtaacaaggtacataaaacttggtcatggtggacactggagtcagagtcatgtagaagttctcttccctacatctgtgagatgacagctttcaggtttccagat

SEQ ID NO: 15 - protein, 655 aa, Artificial Sequence
  Made in a lab
MEAPAQLLFLLLLWLPDTTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPFDSWGQGTLVTVSSGGGGSGGGGSGGGGSGGGGSGGGGSEIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRTEQKLSNMENRLKPFFTCGSADTCCPSGWIMHQKSCFYISLTSKNWQESQKQCETLSSKLATFSEIYPQSHSYYFLNSLLPNGGSGNSYWTGLSSNKDWKLTDDTQRTRTYAQSSKCNKVHKTWSWWTLESESCRSSLPYICEMTAFRFPD

SEQ ID NO: 16 - DNA, 1956 bp, Artificial Sequence
  Made in a lab
atggaagccccagctcagcttctcttcctcctgctactctggctcccagataccaccggagaggtgcagctggtgcagtctggagcagaggtgaaaaagcccggagagtctctgaagatttcctgtaagggctccggttactcattcactggctacaatatgaactgggtgcgccagatgcccgggaaaggcctcgagtggatgggcaatattgatccttattatggtggtactacctacaaccggaagttcaagggccaggtcactatctccgccgacaagtccatcagcaccgcctacctgcaatggagcagcctgaaggcctcggacaccgccatgtattactgtgcacgctcagtcggccctttcgactcctggggccagggcaccctggtcactgtctcctctgggggtggaggctctggtggcggtggctctggcggaggtggatccggtggcggcggatctggcgggggtggctctgaaattgtgttgacacagtctccagccaccctgtctttgtctccaggcgaaagagccaccctctcctgccgagcaagtgaaaatgtttacagctacttagcctggtaccaacagaaacctggccaggctcctaggctcctcatctattttgcaaaaaccttagcagaaggaattccagccaggttcagtggcagtggctccgggacagacttcactctcaccatcagcagcctagagcctgaagattttgcagtttattactgtcaacatcattccgataatccgtggacattcggccaagggaccaaggtggaaatcaaaggtgatcaggagcccaaatcttctgacaaaactcacacatctccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaagcgtacgcagaggcacaacaattcttccctgaatacaagaactcagaaagcacgtcattctggccattgtccgtcgggatggataatgcatcagaaaagctgcttttacatctcacttacttcaaaaaattggcaggagagccaaaaacaatgtgaaactctgtcttccaagctggccacattcagtgaaatttatccacaatcacactcttactacttcttaaattcactgttgccaaatggtggttcagggaattcatattggactggcctcagctctaacaaggattggaagttgactgatgatacacaacgcactaggacttatgctcaaagctcaaaatgtaacaaggtacataaaacttggtcatggtggacactggagtcagagtcatgtagaagttctcttccctacatctgtgagatgacagctttcaggtttccagat

SEQ ID NO: 17 - protein, 652 aa, Artificial Sequence
  Made in a lab
MEAPAQLLFLLLLWLPDTTGEVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPFDSWGQGTLVTVSSGGGGSGGGGSGGGGSGGGGSGGGGSEIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRTQRHNNSSLNTRTQKARHSGHCPSGWIMHQKSCFYISLTSKNWQESQKQCETLSSKLATFSEIYPQSHSYYFLNSLLPNGGSGNSYWTGLSSNKDWKLTDDTQRTRTYAQSSKCNKVHKTWSWWTLESESCRSSLPYICEMTAFRFPD

SEQ ID NO: 18 - protein, 2 aa, Artificial Sequence
  Made in a lab
NS

SEQ ID NO: 19 - protein, 6 aa, Artificial Sequence
  Made in a lab
SCPPCP

SEQ ID NO: 20 - protein, 8 aa, Artificial Sequence
  Made in a lab
GGGGSGNS

SEQ ID NO: 21 - protein, 8 aa, Artificial Sequence
  Made in a lab
GCPPCPNS

SEQ ID NO: 22 - protein, 8 aa, Artificial Sequence
  Made in a lab
GSPPSPNS

SEQ ID NO: 23 - protein, 8 aa, Artificial Sequence
  Made in a lab
GSPPSPNS

SEQ ID NO: 24 - protein, 8 aa, Artificial Sequence
  Made in a lab
GCPPCPNS

SEQ ID NO: 25 - protein, 8 aa, Artificial Sequence
  Made in a lab
GCPPCPNS

SEQ ID NO: 26 - protein, 9 aa, Artificial Sequence
  Made in a lab
GCPPCPGNS

SEQ ID NO: 27 - protein, 9 aa, Artificial Sequence
  Made in a lab
GCPPCPANS

SEQ ID NO: 28 - protein, 9 aa, Artificial Sequence
  Made in a lab
GCPPCPANS

SEQ ID NO: 29 - protein, 9 aa, Artificial Sequence
  Made in a lab
EEEEDEGNS

SEQ ID NO: 30 - protein, 10 aa, Artificial Sequence
  Made in a lab
NYGGGGSGNS

SEQ ID NO: 31 - protein, 10 aa, Artificial Sequence
  Made in a lab
VSERPFPPNS

SEQ ID NO: 32 - protein, 11 aa, Artificial Sequence
  Made in a lab
EPKSCDKTCCP

SEQ ID NO: 33 - protein, 11 aa, Artificial Sequence
  Made in a lab
SQPEIVPISNS

SEQ ID NO: 34 - protein, 12 aa, Artificial Sequence
  Made in a lab
GGGGSCPPCPNS

SEQ ID NO: 35 - protein, 12 aa, Artificial Sequence
  Made in a lab
KADFLTPSIGNS

SEQ ID NO: 36 - protein, 12 aa, Artificial Sequence
  Made in a lab
QMNSELSVLANS

SEQ ID NO: 37 - protein, 13 aa, Artificial Sequence
  Made in a lab
EPKSCDKTHTCCP

SEQ ID NO: 38 - protein, 13 aa, Artificial Sequence
  Made in a lab
EPKSCDKTCPPCP

SEQ ID NO: 39 - protein, 13 aa, Artificial Sequence
  Made in a lab
GGGGSGGGGSGNS

SEQ ID NO: 40 - protein, 13 aa, Artificial Sequence
  Made in a lab
GCPPCPGGGGSNS

SEQ ID NO: 41 - protein, 13 aa, Artificial Sequence
  Made in a lab
GGGGSCPPCPGNS

SEQ ID NO: 42 - protein, 13 aa, Artificial Sequence
  Made in a lab
GCPPCPGGGGSNS

SEQ ID NO: 43 - protein, 13 aa, Artificial Sequence
  Made in a lab
GGGASCPPCPGNS

SEQ ID NO: 44 - protein, 13 aa, Artificial Sequence
  Made in a lab
GGGASCPPCAGNS

SEQ ID NO: 45 - protein, 13 aa, Artificial Sequence
  Made in a lab
GGGASCPPCAGNS

SEQ ID NO: 46 - protein, 15 aa, Artificial Sequence
  Made in a lab
NYGGGGSGGGGSGNS

SEQ ID NO: 47 - protein, 15 aa, Artificial Sequence
  Made in a lab
LSVKADFLTPSIGNS

SEQ ID NO: 48 - protein, 15 aa, Artificial Sequence
  Made in a lab
LSVLANFSQPEIGNS

SEQ ID NO: 49 - protein, 15 aa, Artificial Sequence
  Made in a lab
LKIQERVSKPKISNS

SEQ ID NO: 50 - protein, 15 aa, Artificial Sequence
  Made in a lab
LDVSERPFPPHIQNS

SEQ ID NO: 51 - protein, 15 aa, Artificial Sequence
  Made in a lab
REQLAEVTLSLKANS

SEQ ID NO: 52 - protein, 15 aa, Artificial Sequence
  Made in a lab
RIHQMNSELSVLANS

SEQ ID NO: 53 - protein, 15 aa, Artificial Sequence
  Made in a lab
DTKGKNVLEKIFSNS

SEQ ID NO: 54 - protein, 15 aa, Artificial Sequence
  Made in a lab
LPPETQESQEVTLNS

SEQ ID NO: 55 - protein, 15 aa, Artificial Sequence
  Made in a lab
RIHLNVSERPFPPNS

SEQ ID NO: 56 - protein, 15 aa, Artificial Sequence
  Made in a lab
LSVKADFLTPSIGNS

SEQ ID NO: 57 - protein, 15 aa, Artificial Sequence
  Made in a lab
LSVLANFSQPEIGNS

SEQ ID NO: 58 - protein, 15 aa, Artificial Sequence
  Made in a lab
LSVLANFSQPEIGNS

SEQ ID NO: 59 - protein, 15 aa, Artificial Sequence
  Made in a lab
RIHQMNSELSVLANS

SEQ ID NO: 60 - protein, 15 aa, Artificial Sequence
  Made in a lab
KPFFTCGSADTCPNS

SEQ ID NO: 61 - protein, 15 aa, Artificial Sequence
  Made in a lab
KPFFTCGSADTCPNS

SEQ ID NO: 62 - protein, 15 aa, Artificial Sequence
  Made in a lab
QYNCPGQYTFSMPNS

SEQ ID NO: 63 - protein, 15 aa, Artificial Sequence
  Made in a lab
GGGGSGGGGSGGGGS

SEQ ID NO: 64 - protein, 15 aa, Artificial Sequence
  Made in a lab
EPKSSDKTHTCPPCP

SEQ ID NO: 65 - protein, 15 aa, Artificial Sequence
  Made in a lab
EPKSSDKTHTCPPCS

SEQ ID NO: 66 - protein, 15 aa, Artificial Sequence
  Made in a lab
EPKSCDKTHTSPPCS

SEQ ID NO: 67 - protein, 15 aa, Artificial Sequence
  Made in a lab
EPKSCDKTHTCPPCP

SEQ ID NO: 68 - protein, 15 aa, Artificial Sequence
  Made in a lab
EPKSCDKTHTSPPSS

SEQ ID NO: 69 - protein, 15 aa, Artificial Sequence
  Made in a lab
PRGPTIKPCPPCKCP

SEQ ID NO: 70 - protein, 15 aa, Artificial Sequence
  Made in a lab
EPKSSDKTHTSPPSS

SEQ ID NO: 71 - protein, 15 aa, Artificial Sequence
  Made in a lab
EPKSSDKTHTSPPCS

SEQ ID NO: 72 - protein, 15 aa, Artificial Sequence
  Made in a lab
EPKSSDKTHTCPPSS

SEQ ID NO: 73 - protein, 15 aa, Artificial Sequence
  Made in a lab
EPKSCDKTHTSPPCP

SEQ ID NO: 74 - protein, 15 aa, Artificial Sequence
  Made in a lab
EPKSSDKTHTSPPSP

SEQ ID NO: 75 - protein, 15 aa, Artificial Sequence
  Made in a lab
EPKSSDKTHTSPPCP

SEQ ID NO: 76 - protein, 15 aa, Artificial Sequence
  Made in a lab
EPKSCDKTHTSPPSP

SEQ ID NO: 77 - protein, 15 aa, Artificial Sequence
  Made in a lab
EPKSSDKTHTCPPSP

SEQ ID NO: 78 - protein, 15 aa, Artificial Sequence
  Made in a lab
GGGGCDKTHTCPPCP

SEQ ID NO: 79 - protein, 15 aa, Artificial Sequence
  Made in a lab
EPKSCGGGGGCPPCP

SEQ ID NO: 80 - protein, 15 aa, Artificial Sequence
  Made in a lab
EPKSCDKTHTCGGCP

SEQ ID NO: 81 - protein, 15 aa, Artificial Sequence
  Made in a lab
EPKSCDKTHTCPPCG

SEQ ID NO: 82 - protein, 15 aa, Artificial Sequence
  Made in a lab
EPKSCDKTHTCPPSP

SEQ ID NO: 83 - protein, 15 aa, Artificial Sequence
  Made in a lab
EPKSCDKCHTCPPCP

SEQ ID NO: 84 - protein, 15 aa, Artificial Sequence
  Made in a lab
EPKSCDKTCCCPPCP

SEQ ID NO: 85 - protein, 15 aa, Artificial Sequence
  Made in a lab
EPKSCPPPPPCPPCP

SEQ ID NO: 86 - protein, 15 aa, Artificial Sequence
  Made in a lab
PPPPCDKTHTCPPCP

SEQ ID NO: 87 - protein, 15 aa, Artificial Sequence
  Made in a lab
EPKSCDKTHTCWWCP

SEQ ID NO: 88 - protein, 15 aa, Artificial Sequence
  Made in a lab
EPKSCDWWHTCPPCP

SEQ ID NO: 89 - protein, 15 aa, Artificial Sequence
  Made in a lab
EPKCSDKTHTCPPCP

SEQ ID NO: 90 - protein, 15 aa, Artificial Sequence
  Made in a lab
EPKSDCKTHTCPPCP

SEQ ID NO: 91 - protein, 15 aa, Artificial Sequence
  Made in a lab
EPKSDCWWHTCPPCP

SEQ ID NO: 92 - protein, 15 aa, Artificial Sequence
  Made in a lab
EPKSCDFFHTCPPCP

SEQ ID NO: 93 - protein, 15 aa, Artificial Sequence
  Made in a lab
EPKSCDWWWTCPPCP

SEQ ID NO: 94 - protein, 15 aa, Artificial Sequence
  Made in a lab
EPKSCWWTHTCPPCP

SEQ ID NO: 95 - protein, 15 aa, Artificial Sequence
  Made in a lab
EPWWCDKTHTCPPCP

SEQ ID NO: 96 - protein, 16 aa, Artificial Sequence
  Made in a lab
SQPEIVPISCPPCPNS

SEQ ID NO: 97 - protein, 17 aa, Artificial Sequence
  Made in a lab
TGEPKSSDKTHTCPPCP

SEQ ID NO: 98 - protein, 17 aa, Artificial Sequence
  Made in a lab
EPKSTDKTHTCPPCPNS

SEQ ID NO: 99 - protein, 17 aa, Artificial Sequence
  Made in a lab
EPKSTDKTHTSPPSPNS

SEQ ID NO: 100 - protein, 17 aa, Artificial Sequence
  Made in a lab
EPKSTDKTHTCPPCPNS

SEQ ID NO: 101 - protein, 17 aa, Artificial Sequence
  Made in a lab
EPKSSDKTHTCPPCPNS

SEQ ID NO: 102 - protein, 18 aa, Artificial Sequence
  Made in a lab
EPKSCDKTHTCPGGGPCP

SEQ ID NO: 103 - protein, 18 aa, Artificial Sequence
  Made in a lab
EPKSCDGGGKTHTCPPCP

SEQ ID NO: 104 - protein, 18 aa, Artificial Sequence
  Made in a lab
EPKSCDPPPKTHTCPPCP

SEQ ID NO: 105 - protein, 18 aa, Artificial Sequence
  Made in a lab
EPKSCDKTHTCPPPPPCP

SEQ ID NO: 106 - protein, 18 aa, Artificial Sequence
  Made in a lab
GGGGSGGGGSGGGGSGNS

SEQ ID NO: 107 - protein, 19 aa, Artificial Sequence
  Made in a lab
NYGGGGSGGGGSGGGGSNS

SEQ ID NO: 108 - protein, 19 aa, Artificial Sequence
  Made in a lab
LSVKADFLTPSISPPCPNS

SEQ ID NO: 109 - protein, 19 aa, Artificial Sequence
  Made in a lab
SVLANFSQPEISCPPCPNS

SEQ ID NO: 110 - protein, 20 aa, Artificial Sequence
  Made in a lab
GQRHNNSSLNTRTQKARHSP

SEQ ID NO: 111 - protein, 20 aa, Artificial Sequence
  Made in a lab
LSVLANFSQPEISCPPCPNS

SEQ ID NO: 112 - protein, 20 aa, Artificial Sequence
  Made in a lab
LKIQERVSKPKISCPPCPNS

SEQ ID NO: 113 - protein, 20 aa, Artificial Sequence
  Made in a lab
REQLAEVTLSLKACPPCPNS

SEQ ID NO: 114 - protein, 20 aa, Artificial Sequence
  Made in a lab
RIHQMNSELSVLACPPCPNS

SEQ ID NO: 115 - protein, 20 aa, Artificial Sequence
  Made in a lab
RIHLNVSERPFPPCPPCPNS

SEQ ID NO: 116 - protein, 20 aa, Artificial Sequence
  Made in a lab
NSLFNQEVQIPLTESYCPNS

SEQ ID NO: 117 - protein, 20 aa, Artificial Sequence
  Made in a lab
EEEEDEEDEEDEEEEEDGNS

SEQ ID NO: 118 - protein, 21 aa, Artificial Sequence
  Made in a lab
LDVSERPFPPHIQSCPPCPNS

SEQ ID NO: 119 - protein, 21 aa, Artificial Sequence
  Made in a lab
DTKGKNVLEKIFDSCPPCPNS

SEQ ID NO: 120 - protein, 21 aa, Artificial Sequence
  Made in a lab
LPPETQESQEVTLSCPPCPNS

SEQ ID NO: 121 - protein, 21 aa, Artificial Sequence
  Made in a lab
EPAFTPGPNIELQKDSDCPNS

SEQ ID NO: 122 - protein, 21 aa, Artificial Sequence
  Made in a lab
QRHNNSSLNTRTQKARHCPNS

SEQ ID NO: 123 - protein, 21 aa, Artificial Sequence
  Made in a lab
QRHNNSSLNTRTQKARHSPNS

SEQ ID NO: 124 - protein, 36 aa, Artificial Sequence
  Made in a lab
NYGGGGSGGGGSGGGGSNYGGGGSGGGGSGGGGSNS

SEQ ID NO: 125 - protein, 122 aa, Artificial Sequence
  Made in a lab
RTRYLQVSQQLQQTNRVLEVTNSSLRQQLRLKITQLGQSAEDLQGSRRELAQSQEALQVEQRAHQAAEGQLQACQADRQKTKETLQSEEQQRRALEQKLSNMENRLKPFFTCGSADTCCPNS

SEQ ID NO: 126 - protein, 13 aa, Artificial Sequence
  Made in a lab
GGGASCPPCAGNS

SEQ ID NO: 127 - protein, 39 aa, Artificial Sequence
  Made in a lab
NNYGGGGSGGGGSGGGGSGNYGGGGSGGGGSGGGGSGNS

SEQ ID NO: 128 - protein, 20 aa, Artificial Sequence
  Made in a lab
NYGGGGSGGGGSGGGGSGNS

SEQ ID NO: 129 - protein, 7 aa, Artificial Sequence
  Made in a lab
SPPSPNS

SEQ ID NO: 130 - protein, 17 aa, Artificial Sequence
  Made in a lab
EPTSTDKTHTSPPSPNS

SEQ ID NO: 131 - protein, 17 aa, Artificial Sequence
  Made in a lab
EPTSTDKTHTCPPCPNS

SEQ ID NO: 132 - protein, 19 aa, Artificial Sequence
  Made in a lab
LSVKADFLTPSISPPCPNS

SEQ ID NO: 133 - protein, 12 aa, Artificial Sequence
  Made in a lab
GGGASCPPCANS

SEQ ID NO: 134 - protein, 22 aa, Artificial Sequence
  Made in a lab
RTQRHNNSSLNTRTQKARHSGH

SEQ ID NO: 135 - protein, 118 aa, Artificial Sequence
  Made in a lab
RTRYLQVSQQLQQTNRVLEVTNSSLRQQLRLKITQLGQSAEDLQGSRRELAQSQEALQVEQRAHQAAEGQLQACQADRQKTKETLQSEEQQRRALEQKLSNMENRLKPFFTCGSADTC

SEQ ID NO: 136 - protein, 35 aa, Artificial Sequence
  Made in a lab
RTQSEEQQRRALEQKLSNMENRLKPFFTCGSADTC

SEQ ID NO: 137 - protein, 25 aa, Artificial Sequence
  Made in a lab
RTEQKLSNMENRLKPFFTCGSADTC

SEQ ID NO: 138 - protein, 26 aa, Artificial Sequence
  Made in a lab
SGGGGSGGGGSGGGGSGGGGSGGGGS

SEQ ID NO: 139 - protein, 16 aa, Artificial Sequence
  Made in a lab
GGGGSGGGGSGGGGAS

SEQ ID NO: 140 - protein, 16 aa, Artificial Sequence
  Made in a lab
SEPKSSDKTHTCPPCP

SEQ ID NO: 141 - protein, 18 aa, Artificial Sequence
  Made in a lab
GDQEPKSSDKTHTSPPCP

SEQ ID NO: 142 - protein, 21 aa, Artificial Sequence
  Made in a lab
QRHNNSSLNTGTQMAGHSPNS

SEQ ID NO: 143 - protein, 21 aa, Artificial Sequence
  Made in a lab
QRHNNSSLNTGTQKARHSPNS

SEQ ID NO: 144 - protein, 21 aa, Artificial Sequence
  Made in a lab
QRHNNSSLNTGTQMARHSPNS

SEQ ID NO: 145 - protein, 21 aa, Artificial Sequence
  Made in a lab
QRHNNSSLNTRTQKAGHSPNS

SEQ ID NO: 146 - protein, 21 aa, Artificial Sequence
  Made in a lab
QRHNNSSLNTRTQMAGHSPNS

SEQ ID NO: 147 - protein, 21 aa, Artificial Sequence
  Made in a lab
QRHNNSSLNTRTQMARHSPNS

SEQ ID NO: 148 - protein, 22 aa, Artificial Sequence
  Made in lab
MDFQVQIFSFLLISASVIMSRG

SEQ ID NO: 149 - protein, 23 aa, Artificial Sequence
  Made in lab
MDFQVQIFSFLLISASVIMSRGX

SEQ ID NO: 150 - protein, 20 aa, Artificial Sequence
  Made in lab
MEAPAQLLFLLLLWLPDTTG

SEQ ID NO: 151 - protein, 10 aa, Human
EPKSCDKTHT

SEQ ID NO: 152 - protein, 7 aa, Human
ERKCCVE

SEQ ID NO: 153 - protein, 12 aa, Human
ELKTPLGDTTHT

SEQ ID NO: 154 - protein, 10 aa, Human
EPKSCDTPPP

SEQ ID NO: 155 - protein, 7 aa, Human
ESKYGPP

SEQ ID NO: 156 - protein, 5 aa, Human
CPRCP

SEQ ID NO: 157 - protein, 5 aa, Human
CPSCP

SEQ ID NO: 158 - protein, 17 aa, Human
ELKTPLGDTTHTCPRCP

SEQ ID NO: 159 - protein, 15 aa, Human
EPKSCDTPPPCPRCP

SEQ ID NO: 160 - protein, 34 aa, Human
ESPKAQASSVPTAQPQAEGSLAKATTAPATTRNT

SEQ ID NO: 161 - protein, 24 aa, Human
GRGGEEKKKEKEKEEQEERETKTP

SEQ ID NO: 162 - protein, 19 aa, Human
VPSTPPTPSPSTPPTPSPS

SEQ ID NO: 163 - protein, 6 aa, Human
VPPPPP

SEQ ID NO: 164 - protein, 107 aa, Human
VCSRDFTPPTVKILQSSSDGGGHFPPTIQLLCLVSGYTPGTINITWLEDGQVMDVDLSTASTTQEGELASTQSELTLSQKHWLSDRTYTCQVTYQGHTFEDSTKKCA

SEQ ID NO: 165 - protein, 112 aa, Human
VIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVP

SEQ ID NO: 166 - protein, 5 aa, Human
CPPCP

SEQ ID NO: 167 - DNA, 39 bp, Artificial Sequence
  Made in a lab
ggtaaacgtacgcgctatctgcaggtgtctcagcagctc

SEQ ID NO: 168 - DNA, 40 bp, Artificial Sequence
  Made in a lab
aggtactctagactaatctggaaacctgaaagctgtcatc

SEQ ID NO: 169 - DNA, 25 bp, Artificial Sequence
  Made in a lab
gtctatataagcagagctctctggc

SEQ ID NO: 170 - DNA, 42 bp, Artificial Sequence
  Made in a lab
ctgcagatagcgcgtacgcttacccggagacagggagaggct

SEQ ID NO: 171 - DNA, 37 bp, Artificial Sequence
  Made in a lab
tctccgggtaagcgtacgcgctatctgcaggtgtctc

SEQ ID NO: 172 - DNA, 45 bp, Artificial Sequence
  Made in a lab
gatcttcgaggcggccgctctagactaatctggaaacctgaaagc

SEQ ID NO: 173 - DNA, 39 bp, Artificial Sequence
  Made in a lab
ccgggtaagcgtacgcaaagtgaggagcaacagaggagg

SEQ ID NO: 174 - DNA, 31 bp, Artificial Sequence
  Made in a lab
gggcagggtgtacacctgtggttctcggggc

SEQ ID NO: 175 - DNA, 36 bp, Artificial Sequence
  Made in a lab
ggtaagcgtacggagcagaagctgagcaacatggag

SEQ ID NO: 176 - DNA, 60 bp, Artificial Sequence
  Made in a lab
ggtaagcgtacgcagaggcacaacaattcttccctgaatacaagaactcagaaagcacgt

SEQ ID NO: 177 - DNA, 2214 bp, Artificial Sequence
  Made in a lab
atggaagcaccagcgcagcttctcttcctcctgctactctggctcccagataccaccggtgatattgtgatgatccaggatgaactctccaatcctgtcagttctggagaatcagtttccatctcctgtaggtctagtaagagtctcctagataaggaagggaagacatacttgaattggtttctgcagagaccaggacaatctcctcagctcctgatctatctgatgtccatgcgtgaatcaggagtctcagaccggtttagtggcagtgggtcaggaacagatttcaccctggaaatcagtagagtgaaggctgaggatgtgggtgtgtattactgtcaacaacttgtagagtatccgtggacgttcggtggaggcaccaagctggaaatcaaaggtggcggtggctctggcggaggtggatccggtggcggcggctctcagatccagttggtgcagtctggacctgagctgaagaagcctggagagacagtcaagatctcctgcaaggcttctggttataccttcacagacttttcaatgcactgggtgaggcaggctccaggaaagggtttaaagtggatgggctggataaacactgagactggtgagccaacatatgcagatgacttcaggggacggtttgccttctctttggaaacttctgccagcactgcctatttgcagatcaacaacctcaaaaatgaggacacggctacatatttttgtacctggtctgcttactggggccaagggactctggtcactgtctctgcctcgagcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaagcgtacgcgctatctgcaggtgtctcagcagctccagcagacgaacagggttctggaagtcactaacagcagcctgaggcagcagctccgcctcaagataacgcagctgggacagagtgcagaggatctgcaggggtccaggagagagctggcgcagagtcaggaagcactacaggtggaacagagggctcatcaggcggccgaagggcagctacaggcctgccaggcagacagacagaagacgaaggagaccttgcaaagtgaggagcaacagaggagggccttggagcagaagctgagcaacatggagaacagactgaagcccttcttcacatgcggctcagcagacacctgctgtccgtcgggatggataatgcatcagaaaagctgcttttacatctcacttacttcaaaaaattggcaggagagccaaaaacaatgtgaaactctgtcttccaagctggccacattcagtgaaatttatccacaatcacactcttactacttcttaaattcactgttgccaaatggtggttcagggaattcatattggactggcctcagctctaacaaggattggaagttgactgatgatacacaacgcactaggacttatgctcaaagctcaaaatgtaacaaggtacataaaacttggtcatggtggacactggagtcagagtcatgtagaagttctcttccctacatctgtgagatgacagctttcaggtttccagat

SEQ ID NO: 178 - protein, 738 aa, Artificial Sequence
  Made in a lab
MEAPAQLLFLLLLWLPDTTGDIVMIQDELSNPVSSGESVSISCRSSKSLLDKEGKTYLNWFLQRPGQSPQLLIYLMSMRESGVSDRFSGSGSGTDFTLEISRVKAEDVGVYYCQQLVEYPWTFGGGTKLEIKGGGGSGGGGSGGGGSQIQLVQSGPELKKPGETVKISCKASGYTFTDFSMHWVRQAPGKGLKWMGWINTETGEPTYADDFRGRFAFSLETSASTAYLQINNLKNEDTATYFCTWSAYWGQGTLVTVSASSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRTRYLQVSQQLQQTNRVLEVTNSSLRQQLRLKITQLGQSAEDLQGSRRELAQSQEALQVEQRAHQAAEGQLQACQADRQKTKETLQSEEQQRRALEQKLSNMENRLKPFFTCGSADTCCPSGWIMHQKSCFYISLTSKNWQESQKQCETLSSKLATFSEIYPQSHSYYFLNSLLPNGGSGNSYWTGLSSNKDWKLTDDTQRTRTYAQSSKCNKVHKTWSWWTLESESCRSSLPYICEMTAFRFPD

SEQ ID NO: 179 - DNA, 1965 bp, Artificial Sequence
  Made in a lab
atggaagcaccagcgcagcttctcttcctcctgctactctggctcccagataccaccggtgatattgtgatgatccaggatgaactctccaatcctgtcagttctggagaatcagtttccatctcctgtaggtctagtaagagtctcctagataaggaagggaagacatacttgaattggtttctgcagagaccaggacaatctcctcagctcctgatctatctgatgtccatgcgtgaatcaggagtctcagaccggtttagtggcagtgggtcaggaacagatttcaccctggaaatcagtagagtgaaggctgaggatgtgggtgtgtattactgtcaacaacttgtagagtatccgtggacgttcggtggaggcaccaagctggaaatcaaaggtggcggtggctctggcggaggtggatccggtggcggcggctctcagatccagttggtgcagtctggacctgagctgaagaagcctggagagacagtcaagatctcctgcaaggcttctggttataccttcacagacttttcaatgcactgggtgaggcaggctccaggaaagggtttaaagtggatgggctggataaacactgagactggtgagccaacatatgcagatgacttcaggggacggtttgccttctctttggaaacttctgccagcactgcctatttgcagatcaacaacctcaaaaatgaggacacggctacatatttttgtacctggtctgcttactggggccaagggactctggtcactgtctctgcctcgagcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaagcgtacgcaaagtgaggagcaacagaggagggccttggagcagaagctgagcaacatggagaacagactgaagcccttcttcacatgcggctcagcagacacctgctgtccgtcgggatggataatgcatcagaaaagctgcttttacatctcacttacttcaaaaaattggcaggagagccaaaaacaatgtgaaactctgtcttccaagctggccacattcagtgaaatttatccacaatcacactcttactacttcttaaattcactgttgccaaatggtggttcagggaattcatattggactggcctcagctctaacaaggattggaagttgactgatgatacacaacgcactaggacttatgctcaaagctcaaaatgtaacaaggtacataaaacttggtcatggtggacactggagtcagagtcatgtagaagttctcttccctacatctgtgagatgacagctttcaggtttccagat

SEQ ID NO: 180 - protein, 655 aa, Artificial Sequence
  Made in a lab
MEAPAQLLFLLLLWLPDTTGDIVMIQDELSNPVSSGESVSISCRSSKSLLDKEGKTYLNWFLQRPGQSPQLLIYLMSMRESGVSDRFSGSGSGTDFTLEISRVKAEDVGVYYCQQLVEYPWTFGGGTKLEIKGGGGSGGGGSGGGGSQIQLVQSGPELKKPGETVKISCKASGYTFTDFSMHWVRQAPGKGLKWMGWINTETGEPTYADDFRGRFAFSLETSASTAYLQINNLKNEDTATYFCTWSAYWGQGTLVTVSASSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRTQSEEQQRRALEQKLSNMENRLKPFFTCGSADTCCPSGWIMHQKSCFYISLTSKNWQESQKQCETLSSKLATFSEIYPQSHSYYFLNSLLPNGGSGNSYWTGLSSNKDWKLTDDTQRTRTYAQSSKCNKVHKTWSWWTLESESCRSSLPYICEMTAFRFPD

SEQ ID NO: 181 - DNA, 1935 bp, Artificial Sequence
  Made in a lab
atggaagcaccagcgcagcttctcttcctcctgctactctggctcccagataccaccggtgatattgtgatgatccaggatgaactctccaatcctgtcagttctggagaatcagtttccatctcctgtaggtctagtaagagtctcctagataaggaagggaagacatacttgaattggtttctgcagagaccaggacaatctcctcagctcctgatctatctgatgtccatgcgtgaatcaggagtctcagaccggtttagtggcagtgggtcaggaacagatttcaccctggaaatcagtagagtgaaggctgaggatgtgggtgtgtattactgtcaacaacttgtagagtatccgtggacgttcggtggaggcaccaagctggaaatcaaaggtggcggtggctctggcggaggtggatccggtggcggcggctctcagatccagttggtgcagtctggacctgagctgaagaagcctggagagacagtcaagatctcctgcaaggcttctggttataccttcacagacttttcaatgcactgggtgaggcaggctccaggaaagggtttaaagtggatgggctggataaacactgagactggtgagccaacatatgcagatgacttcaggggacggtttgccttctctttggaaacttctgccagcactgcctatttgcagatcaacaacctcaaaaatgaggacacggctacatatttttgtacctggtctgcttactggggccaagggactctggtcactgtctctgcctcgagcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaagcgtacggagcagaagctgagcaacatggagaacagactgaagcccttcttcacatgcggctcagcagacacctgctgtccgtcgggatggataatgcatcagaaaagctgcttttacatctcacttacttcaaaaaattggcaggagagccaaaaacaatgtgaaactctgtcttccaagctggccacattcagtgaaatttatccacaatcacactcttactacttcttaaattcactgttgccaaatggtggttcagggaattcatattggactggcctcagctctaacaaggattggaagttgactgatgatacacaacgcactaggacttatgctcaaagctcaaaatgtaacaaggtacataaaacttggtcatggtggacactggagtcagagtcatgtagaagttctcttccctacatctgtgagatgacagctttcaggtttccagat

SEQ ID NO: 182 - protein, 645 aa, Artificial Sequence
  Made in a lab
MEAPAQLLFLLLLWLPDTTGDIVMIQDELSNPVSSGESVSISCRSSKSLLDKEGKTYLNWFLQRPGQSPQLLIYLMSMRESGVSDRFSGSGSGTDFTLEISRVKAEDVGVYYCQQLVEYPWTFGGGTKLEIKGGGGSGGGGSGGGGSQIQLVQSGPELKKPGETVKISCKASGYTFTDFSMHWVRQAPGKGLKWMGWINTETGEPTYADDFRGRFAFSLETSASTAYLQINNLKNEDTATYFCTWSAYWGQGTLVTVSASSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRTEQKLSNMENRLKPFFTCGSADTCCPSGWIMHQKSCFYISLTSKNWQESQKQCETLSSKLATFSEIYPQSHSYYFLNSLLPNGGSGNSYWTGLSSNKDWKLTDDTQRTRTYAQSSKCNKVHKTWSWWTLESESCRSSLPYICEMTAFRFPD

SEQ ID NO: 183 - DNA, 1926 bp, Artificial Sequence
  Made in a lab
atggaagcaccagcgcagcttctcttcctcctgctactctggctcccagataccaccggtgatattgtgatgatccaggatgaactctccaatcctgtcagttctggagaatcagtttccatctcctgtaggtctagtaagagtctcctagataaggaagggaagacatacttgaattggtttctgcagagaccaggacaatctcctcagctcctgatctatctgatgtccatgcgtgaatcaggagtctcagaccggtttagtggcagtgggtcaggaacagatttcaccctggaaatcagtagagtgaaggctgaggatgtgggtgtgtattactgtcaacaacttgtagagtatccgtggacgttcggtggaggcaccaagctggaaatcaaaggtggcggtggctctggcggaggtggatccggtggcggcggctctcagatccagttggtgcagtctggacctgagctgaagaagcctggagagacagtcaagatctcctgcaaggcttctggttataccttcacagacttttcaatgcactgggtgaggcaggctccaggaaagggtttaaagtggatgggctggataaacactgagactggtgagccaacatatgcagatgacttcaggggacggtttgccttctctttggaaacttctgccagcactgcctatttgcagatcaacaacctcaaaaatgaggacacggctacatatttttgtacctggtctgcttactggggccaagggactctggtcactgtctctgcctcgagcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaagcgtacgcagaggcacaacaattcttccctgaatacaagaactcagaaagcacgtcattctggccattgtccgtcgggatggataatgcatcagaaaagctgcttttacatctcacttacttcaaaaaattggcaggagagccaaaaacaatgtgaaactctgtcttccaagctggccacattcagtgaaatttatccacaatcacactcttactacttcttaaattcactgttgccaaatggtggttcagggaattcatattggactggcctcagctctaacaaggattggaagttgactgatgatacacaacgcactaggacttatgctcaaagctcaaaatgtaacaaggtacataaaacttggtcatggtggacactggagtcagagtcatgtagaagttctcttccctacatctgtgagatgacagctttcaggtttccagat

SEQ ID NO: 184 - protein, 642 aa, Artificial Sequence
  Made in a lab
MEAPAQLLFLLLLWLPDTTGDIVMIQDELSNPVSSGESVSISCRSSKSLLDKEGKTYLNWFLQRPGQSPQLLIYLMSMRESGVSDRFSGSGSGTDFTLEISRVKAEDVGVYYCQQLVEYPWTFGGGTKLEIKGGGGSGGGGSGGGGSQIQLVQSGPELKKPGETVKISCKASGYTFTDFSMHWVRQAPGKGLKWMGWINTETGEPTYADDFRGRFAFSLETSASTAYLQINNLKNEDTATYFCTWSAYWGQGTLVTVSASSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRTQRHNNSSLNTRTQKARHSGHCPSGWIMHQKSCFYISLTSKNWQESQKQCETLSSKLATFSEIYPQSHSYYFLNSLLPNGGSGNSYWTGLSSNKDWKLTDDTQRTRTYAQSSKCNKVHKTWSWWTLESESCRSSLPYICEMTAFRFPD